(19) United States
(12) Patent Application Publication
(10) Pub. No.: US 2018/0163196 A1
(43) Pub. Date: Jun. 14, 2018

(54) CRISPR/CAS9 BASED ENGINEERING OF ACTINOMYCETAL GENOMES

(71) Applicant: Danmarks Tekniske Universitet, Kgs. Lyngby (DK)

(72) Inventors: Tilmann Weber, Fredensborg (DK); Yaojun Tong, Hørsholm (DK); Sang Yup Lee, Daejeon (KR)

(21) Appl. No.: 15/559,753

(22) PCT Filed: Mar. 18, 2016
(86) PCT No.: PCT/EP2016/055967

§ 371 Date: Sep. 19, 2017

(30) Foreign Application Priority Data
Mar. 20, 2015 (EP) 15160126.7

(51) Int. Cl. C12N 15/10; C12N 1/20

(52) U.S. Cl. CPC C12N 15/102; C12N 9/22; C12N 2310/20; C12N 1/20

(57) ABSTRACT

The present invention relates to CRISPR/Cas-based methods for generating random-sized deletions around at least one target nucleic acid sequence, or for generating precise indels around at least one target nucleic acid sequence, or for modulating transcription of at least one target nucleic acid sequence. Also disclosed is a clonal library comprising clones with random-sized deletions, as well as polynucleotides, polypeptides, cells and kits useful for performing the present methods. The present methods can be performed in organisms where gene editing is typically considered as difficult, such as actinomycetes, in particular streptomycetes.

### FIELD OF INVENTION

The present invention relates to CRISPR/Cas-based methods for generating random-sized deletions around at least one target nucleic acid sequence, or for generating precise indels around at least one target nucleic acid sequence, or for modulating transcription of at least one target nucleic acid sequence. Also disclosed is a clonal library comprising clones with random-sized deletions, as well as polynucleotides, polypeptides, cells and kits useful for performing the present methods. The present methods can be performed in organisms where gene editing is typically considered as difficult, such as actinomycetes, in particular streptomycetes.

### BACKGROUND OF INVENTION

Actinomycetes are Gram-positive bacteria with the capacity to produce a wide variety of medically and industrially relevant secondary metabolites, including many antibiotics, herbicides, parasiticides, anti-cancer agents, and immunosuppressants. It becomes harder and harder to find new bioactive compounds from actinomycetes using traditional approaches.

Recent advances in genome sequencing and genome mining have significantly accelerated the ability to identify secondary metabolism genes and gene clusters. Precise gene editing technologies are needed to enable systematic reverse engineering of causal genetic variations by allowing selective perturbation of individual genetic elements, as well as to advance synthetic biology and biotechnology. There are four major universal gene editing tools developed so far: 1) meganucleases derived from microbial mobile genetic elements, 2) zinc finger (ZF) nucleases based on eukaryotic transcription factors, 3) transcription activator-like effectors (TALEs) from Xanthomonas bacteria, and 4) the RNA-guided DNA endonuclease Cas9 from the type II bacterial adaptive immune system Clustered Regularly Interspaced Short Palindromic Repeats (CRISPR), called CRISPR-Cas9 system. However, each of the first three methods has its own unique limitations: the specificity of a meganuclease for a target DNA is difficult to control, the assembly of functional zinc finger proteins with the desired DNA binding specificity remains a major challenge, and the construction of novel TALE arrays are labour intensive and costly.

The CRISPR-Cas9 system displays certain advantages. The CRISPR nuclease Cas9 can be guided by a short single guide RNA (sgRNA) that recognizes the target DNA via Watson-Crick base pairing (FIG. 1A) instead of complex protein-DNA recognition, thereby easing the design and construction of targeting vectors. The sgRNAs are artificially generated chimeras of the CRISPR RNA (crRNA) and the associated trans-activating CRISPR RNA (tracrRNA) found in the native CRISPR systems, which originally corresponds to phage sequences, constituting the natural mechanism for CRISPR antiviral defense of bacteria and archaea, but can be easily replaced by a sequence of interest to reprogram the Cas9 nuclease for gene editing. Multiplexed targeting by Cas9 can now be achieved at an unprecedented scale by introducing a plurality of sgRNAs rather than a library of large, bulky proteins.

The Cas9 protein family is characterized by two signature nuclease domains, HNH and RuvC. A critical feature of recognition by CRISPR-Cas9 is the protospacer-adjacent motif (PAM), which flanks the 3′ end of the DNA target site (FIG. 1) and directs the DNA target recognition by the Cas9-sgRNA complex. The Cas9 and the sgRNA first form a complex, and the complex subsequently starts to scan the whole genome for the PAM sequences. Once the complex has identified the PAM, which can have on its 5′ flank a sequence complementary to the target sequence within the sgRNA in the complex, the complex binds to this position. This triggers the Cas9 nuclease activity by activating the HNH and RuvC domains.

The CRISPR/Cas9 system generates a break, such as a nick or a double-strand break (DSB) in the DNA, which is repaired by one of the two main repair pathways: non-homologous end-joining (NHEJ) or homologous recombination (HR). HR requires the presence of a homologous template DNA, which can comprise additional sequences which can thus be introduced at the site of the break. NHEJ does not require the presence of donor DNA, and usually results in small deletions. The system can thus be used for integrating new sequences into a target sequence, or for the precise generation of deletions around the target site.

Because of its modularization and easy handling, the CRISPR-Cas9 system has been successfully applied as a gene editing tool in a wide range of organisms such as Saccharomyces cerevisiae, some plants, Caenorhabditis elegans, Drosophila, Chinese hamster ovary (CHO) cells, frogs, mice, rats, rabbits, and human cells with high specificity. Recently, the CRISPR-Cas9 system was re-programmed to control gene expression by mutating the HNH and RuvC domains of Cas9 (D10A and H840A), resulting in a catalytically dead Cas9 (dCas9) lacking endonuclease activity. This system has so far successfully been applied in Escherichia coli (Qi, L. S., et, al. 2013).

As stated above, one of the challenges in the deep application of actinomycetes is to systematically engineer them for the overproduction of effective secondary metabolites and non-natural chemical compounds as well as new bioactive compounds, which corresponds to a fundamental objective of metabolic engineering. Unfortunately, genetic manipulation of actinomycetes is considered to be more difficult than model organisms, such as Escherichia coli and Saccharomyces cerevisiae. This is due in part to their more diverse genomic contents; for example, the GC content of their genomes is high.

There are to our knowledge only two very recent publications describing a CRISPR based system using homologous recombination templates to generate defined mutations in streptomycetes (Cobb et al., 2014, Huang et al., 2015). The use of CRISPR-based systems for generating random-sized, targeted deletions around a target site has not yet been reported.

Thus, rapid, efficient and convenient methods for gene editing of actinomycetes, in particular for streptomycetes, are needed.

### SUMMARY OF INVENTION

The invention is as defined in the claims.

Herein are disclosed methods useful for gene editing. These methods are based on the surprising finding that in organisms having a partly deficient non-homologous end-joining pathway (NHEJ), gene editing based on the CRISPR/Cas9 system targeting a nucleic acid sequence of interest results in the generation of clones with random-sized deletions around the target site. In order to generate precise indels (i.e. precise insertions or deletions) around a target site in such organisms, the NHEJ pathway can be restored by engineering the host cell so that it has a fully functional NHEJ pathway.

The methods described herein are of particular interest for organisms where gene editing is typically considered to be labor-intensive, such as actinomycetes. The methods can be used to generate clonal libraries in order to investigate a given pathway, for example in order to optimize production of a secondary metabolite.

Also described herein is a method for modulating transcription of a nucleic acid sequence of interest by using a catalytically dead Cas9. This method can be applied to actinobacteria, e.g. streptomycetes.

### DESCRIPTION OF DRAWINGS

FIG. 1. Diagram of the Cas9 and sgRNA complex. The Cas9 HNH and RuvC-like domains each cleave one strand of the sequence targeted by the sgRNA; the trinucleotide PAM is labelled; the binding of the 20 nt target sequence to the genome is shown; the sgRNA core structure and sequence is shown.

FIG. 2. Design of easily changeable sgRNA scaffold: the forward primer, labelled as “P-F”, comprises a 20 nt sgRNA core sequence, a 20 nt target sequence and the NcoI sequence, while the reverse primer, labelled as “P-R”, comprises a 20 nt sgRNA core sequence and the SnaBI sequence. To construct a new sgRNA, a 20 nt target sequence of interest is designed and integrated in the forward primer. The arrow represents the ermE* promoter, while the circle represents the to terminator, and the core sgRNA is shown as a box.

FIG. 3. Map of pCRISPR-Cas9. Restriction endonuclease sites are available for additional elements sub-cloning, for instance, the Stul site.

FIG. 4. Actinorhodin biosynthesis. A. Organization of the actinorhodin biosynthetic gene cluster; B. The steps to synthetize actinorhodin are: I. 1× Acetyl-CoA and 7× malonyl-CoA are condensed to form the carbon skeleton by ActI; II. The above carbon backbone is cyclized to form a three ring intermediate, DNPA by ActIII, ActVII, ActIV, ActVI-1 and ActVI-3; III. DNPA is then modified to form DHK by ActVI-2, ActVI-4 and ActVA-6; IV. 2 DHK is dimerized to form the final product, actinorhodin by ActVA-5 and ActVB. The arrows mark the two selected genes.

FIG. 5. Functional sgRNAs PCR screening results: the positive size is 234 bp, the negative size is 214 bp, the agrose gel concentration is 4% in TAE. A-C, 36 clones for actlORF1 gene; D-F, 36 clones for actVB gene.

FIG. 6. Actinorhodin biosynthetic pathway was inactivated by CRISPR-Cas9. 1-5, represent strains WT, Δactlorf1-1, Mismatch, Δactvb-1, and No Target, respectively; the plate in the left panel is without inducer thiostrepton, while the plate in the right panel is with inducer thiostrepton, the pH of the plates is >7. A. ISP2 plate without antibiotics. All five strains are blue. B. ISP2 plate with 1 μg/ml thiostrepton. Labels correspond to those in B. The blue from strains Δactlorf1-1 and Δactvb-1 disappeared. The photos were taken after 7 days incubation at 30° C.

FIG. 7. Actinorhodin detection by UV-visible spectrometry. When the pH is lowered to 2, actinorhodin turns from blue to red, and has a maximum absorption at about 530 nm. From the scanning, the actinorhodin peak of Δactlorf1 and Δactvb disappeared.

FIG. 8. Analysis of the sequencing data. A. Heatmap of the 7 mapped sequencing samples to the S. coelicolor A3(2) reference genome. Dark colours represent a high read coverage, white represents low/no coverage. Displayed is the region spanning 5508800 to 5557230 of the S. coelicolor genome. The actinorhodin gene cluster is denoted by brackets; the target sites of the actlORF1 and actVB sgRNAs are displayed as arrows. The deletion sizes are shown on the map. 1-7 represent strains: WT, No Target, Mismatch, Δactlorf1-1, Δactlorf1-2, Δactvb-1, and Δactvb-2, respectively. B. Alignment of the sequence traces of Δactlorf1-1 with the WT. The arrow indicates the genomic target site of the sgRNA: ActIorf1-6 T. The PAM sequence is shown. C. and D. DNA sequences of 8 randomly selected clones without actinorhodin production aligned to the WT genomic sequence of actlORF1 and actVB, respectively. The arrow indicates the genomic target sites of the related sgRNAs. The PAM sequences are shown. Dark shadow, light shadow with a dash and dark shadow with a box indicate insertions, deletions and substitutions, respectively.

FIG. 9. Plasmid map for pCRISPR-Cas9-ScaligD. An expression cassette of S. carneus ligD was introduced into pCRISPR-Cas9 using Gibson Assembly in Stul site. The S. carneus ligD was under control by ermE* promoter, ending with a to terminator.

FIG. 10. HDR pathway to repair the DNA DSBs caused by CRISPR-Cas9 system. A. and B. Diagrams of the CRISPR-Cas9 vectors with homologous recombination templates for actlORF1 and actVB. C. and D. Colony PCR of 10 randomly selected clones that lost actinorhodin production to confirm deletion of actlORF1 (C) and actVB (D) after use of the two vectors in A and B. I, II, and III represent the WT genome, actlORF1 deleted and actVB deleted genome, respectively. 1-10 represent 10 randomly selected clones that lost actinorhodin production.

FIG. 11. The plasmid map for pCRISPR-dCas9. The only difference between pCRISPR-dCas9 and pCRISPR-Cas9 is the Cas9 was a catalytically dead version without the endonuclease activity (D10A and H840A), called dCas9 in pCRISPR-dCas9.

FIG. 12. CRISPRi effectively silences actlORF1 expression in a reversible manner. A. Location of the twelve sgRNAs for CRISPRi. Half were designed to target the pro-moter region, while the other half were designed to target the ORF. In addition, half target the template strand and half target the non-template strand. The dashes represent sgRNAs. B. 530 nm absorbance of extracts from cultures tested with the twelve sgRNAs shown in A relative to the wild-type control. Left panel shows the sgRNAs target on promoter region, while right panel shows the sgRNAs target on ORF region. Mean values from three independent extractions are shown. Error bars represent the standard deviation from three independent extractions. C. and D. Reversibility of the CRISPRi system. Red clones become blue when the incubation temperature is increased to 37° C., indicating that the CRISPRi effect has gone away. The red color is boxed, while the blue is not. 0-12 represent sgRNAs: control (without any sgRNA), orf1p-A1 NT, orf1p-A4 NT, orf1p-A5 NT, orf1p-S1 T, orf1p-S3 T, orf1p-S5 T, ActIorf1-1 NT, ActIorf1-7 NT, ActIorf1-8 NT, ActIorf1-2 T, ActIorf1-3 T, and ActIorf1-4 T, respectively.

### DETAILED DESCRIPTION OF THE INVENTION

The present inventors have surprisingly found that a partial deficiency of the non-homologous end-joining (NHEJ) pathway in a host cell conferred the host cell interesting properties. For example, inducing a CRISPR-Cas9 system in said host cell results in the generation of random-sized deletions around a target site recognized by said CRISPR-Cas9 system. On the other hand, restoring full functionality of the NH EJ pathway prior to or simultaneously with induction of the CRISPR-Cas9 system results in the generation of precise indels around the target site.

In a first aspect, the invention relates to a method for generating at least one deletion around at least one target nucleic acid sequence comprised within a host cell having a non-homologous end-joining (NHEJ) pathway which is at least partly deficient,
said method comprising the steps of:
(i) optionally, restoring the full functionality of the NHEJ pathway,
(ii) inducing a CRISPR-Cas9 system in said host cell, wherein said CRISPR-Cas9 system is able to generate at least one break in said at least one target nucleic acid sequence and wherein the CRISPR-Cas9 system comprises a Cas9 nuclease and at least one guiding means,
thereby generating:
a. if the method does not comprise step (i)., at least one random-sized deletion around said at least one target nucleic acid sequence, wherein said at least one deletion is a random-sized deletion of at least 1 bp; or
b. if the method does comprise step (i), at least one indel around said at least one target nucleic acid sequence, wherein said at least one indel is a deletion or insertion of at least 1 bp.

In a second aspect, the invention relates to a polynucleotide having at least 94% identity with SEQ ID NO: 1, such as at least 95% identity, such as at least 96% identity, such as at least 97% identity, such as at least 98% identity, such as at least 99% identity, such as 100% identity with SEQ ID NO: 1.

In yet another aspect, the invention relates to a polypeptide encoded by the polynucleotide described herein.

In yet another aspect, the invention relates to a cell comprising the polynucleotide described herein.

In yet another aspect, the invention relates to a cell comprising the polypeptide described herein.

In yet another aspect, the invention relates to a vector comprising the polynucleotide described herein.

In yet another aspect, the invention relates to a clonal library obtainable by the above method, said clonal library comprising a plurality of clones harboring at least one deletion and/or indel around at least one target nucleic acid sequence, wherein said deletion is a random-sized deletion of at least 1 bp and wherein said indel is a deletion or insertion of at least 1 bp.

In yet another aspect, the invention relates to a method for selectively modulating transcription of at least one target nucleic acid sequence in a host cell, the method comprising introducing into the host cell:
i. at least one guiding means, or a nucleic acid comprising a nucleotide sequence encoding guiding means, wherein the guiding means comprises a nucleotide sequence that is complementary to a target nucleic acid sequence in the host cell; and
ii. a variant Cas9, or a nucleic acid comprising a nucleotide sequence encoding the variant Cas9, wherein the variant Cas9 is the polypeptide described herein, or wherein the nucleotide sequence encoding the variant Cas9 is the polynucleotide described herein, and wherein the variant Cas9 has reduced endodeoxyribonuclease activity,

wherein said guiding means and said variant Cas9 form a complex in the host cell, said complex selectively modulating transcription of at least one target nucleic acid in the host cell.

In yet another aspect, the invention relates to a clonal library obtainable by the methods disclosed herein, said clonal library comprising a plurality of clones harbouring at least one deletion and/or indel around at least one target nucleic acid sequence, wherein said deletion is a random-sized deletion of at least 1 bp and wherein said indel is a deletion or insertion of at least 1 bp.

In yet another aspect, the invention relates to a kit for performing the method of the first aspect, said kit comprising a vector comprising a nucleic acid sequence encoding a Cas9 nuclease or a variant thereof, and instructions for use.

In yet another aspect, the invention relates to a kit for performing the method of the second aspect, said kit comprising a vector comprising a variant Cas9, or a nucleic acid comprising a nucleotide sequence encoding the variant Cas9, wherein the variant Cas9 is the polypeptide of claim 4 or the nucleotide sequence encoding the variant Cas9 is the polynucleotide of claim 3, and wherein the variant Cas9 has reduced endodeoxyribonuclease activity, and instructions for use.

Definitions

Break: the term ‘break’ shall be construed as referring to a double strand break, a single strand break or a nick in a DNA strand.

Cluster or gene cluster: these terms refer to a group of closely linked genes that are collectively responsible for a multi-step process such as the biosynthesis of a metabolite, for example a secondary metabolite.

CRISPR-Cas9 system: the terms ‘CRISPR-Cas9’, ‘CRISPR/Cas9’ and ‘type II CRISPR’ and systems thereof will be used interchangeably and refer to a system comprising a CRISPR-Cas9 protein and at least one guiding means, so that the CRISPR-Cas9 system is capable, when induced, of generating at least one break in at least one target nucleic acid sequence. Thus a CRISPR-Cas9 system herein comprises Cas9 and at least one guiding means. The guiding means are as defined below.

Deletion: the term ‘deletion’ refers to the deletion of one or more nucleotides or base pairs in a nucleic acid sequence. The term ‘precise deletion’ refers to smaller deletions, while the term ‘random-sized deletion’ refers to deletions of at least 1 bp which can span over several kilobases, as detailed below.

Double strand break (DSB): a double strand break (DSB) as understood herein refers to a break on both strands of a nucleic acid. DSBs are particularly hazardous to the cell because they can lead to genome rearrangements. Two major mechanisms exist to repair DSBs: non-homologous end joining (NHEJ) and homologous recombination (HR). The choice of pathway depends on parameters such as the nature of the organism and the cell cycle phase.

Enhancers: enhancers are cis-acting elements that can regulate transcription from nearby genes and function by acting as binding sites for transcription factors.

Gene: A gene as understood herein refers to a gene or a putative gene. The gene may code for a selection marker, a protein of interest, a peptide, a secondary metabolite, or it may be a gene resulting in the production of a miRNA, a siRNA, a tRNA, or any gene which can be transcribed and/or translated.

Guiding means: in the present context, the term refers to an element capable of guiding a nuclease such as Cas9 towards its target. Guiding means can be for example a single guide RNA (sgRNA) or a crRNA/tracrRNA set.

Homologous Recombination (HR): Homologous Recombination is one of the two major pathways for repairing DSBs. HR is a type of genetic recombination in which nucleotide sequences are exchanged between two similar or identical molecules of DNA. HR involves copying information from a donor DNA. The terms HR and HDR (homology-directed repair) are herein used interchangeably.

Homology arm or homologous recombination (HR) template: the term covers a stretch of DNA with sequences homologous to the upstream and downstream regions of a region of interest, in particular of a cut site or a targeted endonuclease site.

Indel: an indel refers to a mutation class, resulting in an insertion and/or a deletion of nucleotides, leading to a net change in the total number of nucleotides. The change in the total number of nucleotides is typically in the range of 1 to 5 nucleotides, but may be up to 100 nucleotides or more.

Knockdown: the term refers to the process by which genes transcription levels can be reduced in an organism.

Knockin: the term refers to the process by which genes can be inserted in a genome. The inserted genes may be genes from the same organism or from other species.

Knockout: the term refers to the process by which genes can be inactivated in an organism, for example by deletion or mutation of part or all of the gene, or of part or all of the elements necessary for the gene to be expressed in a functional protein.

Multiplex editing: the term refers herein to editing nucleic acid sequences of multiple sequences, which can be performed simultaneously or serially. For example, multiplex editing may refer to serial knockins and/or serial knockouts or a combination of knockins and knockouts. It may also refer to simultaneous knockins and/or knockouts of multiple target nucleic acid sequences.

Nick: a nick is a discontinuity in a double-stranded DNA molecule where there is no phosphodiester bond between adjacent nucleotides of one strand.

Non-Homologous End Joining (NHEJ): NHEJ is one of the two major pathways for repairing DSBs. The NHEJ pathway harbours four NHEJ activities defined below, which usually involve at least one Ku protein and a ligase. The two ends at the break are joined directly. The ends at the break may be resected prior to repair, which may lead to loss of some nucleotides and improper repair. Thus NH EJ is often error-prone.

NHEJ activity: the term ‘activity’ as used herein may refer to a protein activity such as an enzymatic activity involved in the NHEJ pathway. In particular, the term is used to refer to a domain, a peptide or a protein capable of acting as a ligase, or as a polymerase, or as a primase, or as a protein capable of binding DNA ends around a break. The DNA binding activity is typically performed by one or more Ku proteins. The ligase and primase activities can be performed by a single protein, such as ligase D. Ligase D can however also be capable of performing only one of the primase or ligase or polymerase activities. A fully functional NHEJ pathway comprises all four activities, while a partly functional or partly deficient NHEJ lacks at least one of these four activities.

Nuclear Localisation Sequence (NLS): a nuclear localisation signal or sequence (NLS) is an amino acid sequence which ‘tags’ a protein for import into the cell nucleus by nuclear transport. Typically, this signal consists of one or more short sequences of positively charged lysines or arginines exposed on the protein surface. Different nuclear localised proteins may share the same NLS. An NLS has the opposite function of a nuclear export signal, which targets proteins out of the nucleus.

Nucleic acid: the term refers herein to a sequence of nucleotides.

Parasiticide: the term is to be understood in its broadest sense as an agent capable of inactivating or killing any undesirable organism and thus comprises insecticides, anthelmintic compounds, larvacides, antiparasitic agents and antiprotozoal agents.

Polynucleotide/Oligonucleotide: the terms “polynucleotide” and “oligonucleotide” as used herein denote a nucleic acid chain. Throughout this application, nucleic acids are designated starting from the 5′-end.

Promoter: a promoter is a DNA sequence near the beginning of a gene (typically upstream) that signals the RNA polymerase where to initiate transcription. Eukaryotic promoters may comprise regulatory elements several kilobases upstream of the gene and typically bind transcription factors involved in the formation of the transcriptional complex. Promoters may be inducible, i.e. their activity may be induced by the presence or absence of a biotic or abiotic compound.

Recognition: as understood herein, the term ‘recognition’ refers to the ability of a molecule to identify a nucleotide sequence. Certain enzymes may require the presence of additional recognition means, such as guiding RNAs or DNA binding domains, to efficiently recognise their substrate sequence. For example, an enzyme or a DNA binding domain may recognise a nucleic acid sequence as a potential substrate and bind to it. Guiding means such as sgRNAs or crRNA/tracrRNA sets may recognise a specific sequence to which they are at least partly homologous.

Recombinase: as understood herein, the term ‘recombinase’ refers to an enzyme that can catalyse directionally sensitive DNA exchange reactions between short (30-40 nucleotides) target site sequences. These reactions enable four basic functional modules, excision/insertion, inversion, translocation and cassette exchange.

Terminator: a terminator is a DNA sequence near the end of a gene (typically downstream) that signals the RNA polymerase where to stop transcription. Eukaryotic terminators are recognized by protein factors and termination is followed by polyadenylation of the mRNA.

CRISPR-Cas9 System

The invention relates to methods for gene editing around or modulation of the transcription of at least one target nucleic acid sequence in a host cell based on the use of a CRISPR-Cas9 system. The terms ‘target nucleic acid sequence’ and ‘target sequence’ will be used interchangeably.

It will be understood that throughout this document, the term ‘CRISPR-Cas9’ system refers to a system comprising a CRISPR-Cas9 protein and at least one guiding means, so that the CRISPR-Cas9 system is capable of recognising at least one target nucleic acid sequence. In some embodiments, the CRISPR-Cas9 system is capable of generating a break in the target nucleic acid sequence, such as a nick on one of the two strands or a double-strand break. Thus the CRISPR-Cas9 system herein comprises Cas9 and at least one guiding means, where the guiding means is capable of directing Cas9 to its target nucleic acid sequence. The guiding means may be any guiding means known in the art and suitable for this purpose. In some embodiments, the guiding means is a single guide RNA. In other embodiments, the guiding means is a set of a crRNA and a tracrRNA. The skilled person knows how to design guiding means which direct the CRISPR-Cas9 system to a desired target nucleic acid sequence.

The nucleic acid sequence encoding Cas9 may be present in the genome of the host cell, e.g. on a chromosome of the host cell, or it may be present on a vector comprised within the host cell. Likewise, the guiding means may be present in the genome of the host cell, e.g. on a chromosome of the host cell, or it may be present on a vector comprised within the host cell. The term ‘present in the genome of the host cell’ means that either the Cas9 gene or the guiding means are naturally present in the genome of the host cell or that they has been introduced e.g. by genome editing and conventional transformation.

In embodiments where the nucleic acid sequence encoding Cas9 and the guiding means are comprised within a vector, Cas9 and the guiding means may be comprised within the same vector. In embodiments where the guiding means are comprised within a vector and the guiding means is a crRNA and a tracrRNA, the nucleic acid sequences for the crRNA and the tracrRNA may be comprised within two different vectors. The nucleic acid sequence encoding Cas9 may then be comprised within one of these two vectors, within a third vector or within the genome of the host cell.

The CRISPR-Cas9 system used for the methods disclosed herein may be capable of generating a break in at least one target nucleic acid sequence, such as in at least two target nucleic acid sequences, such as in at least three target nucleic acid sequences, such as in at least four target nucleic acid sequences, such as in at least five target nucleic acid sequences. The CRISPR-Cas9 system can thus be used for multiplex editing.

The skilled person knows how to adapt the CRISPR-Cas9 system recognising more than one target nucleic acid sequence. By way of illustration, the system may comprise two different sgRNAs that each target one target nucleic acid sequence when recognition of two target nucleic acid sequences is desired, or the system may comprise one sgRNA targeting a first target nucleic acid sequence and a crRNA and tracrRNA targeting a second target nucleic acid sequence. Where editing of three target sequences is desired, three different sgRNAs can be used, or two different sgRNAs each targeting a first and a second target sequence and a crRNA and tracrRNA targeting a third sequence, or one sgRNA targeting a first sequence and two sets of crRNA and tracrRNA each targeting a second and a third sequence, or three sets of crRNA and tracrRNA each targeting a different target sequence.

The sequences of the nucleic acid(s) encoding the elements of the CRISPR-Cas9 system may be codon-optimized depending on the host cell in which gene editing is to be performed. Methods for codon optimization are known in the art.

Host Cell

The methods of the present invention allow editing of at least one target nucleic acid sequence comprised within a host cell.

The present method can be performed in an archaea, in a prokaryotic cell or in a eukaryotic cell. In one embodiment, the host cell is a prokaryotic cell. The present methods are particularly advantageous for gene editing in host cells that have a high GC content and where gene editing can be difficult to perform. In some embodiments, the GC content is higher than 50% or more, such as 55% or more, such as 60% or more, such as 65% or more, such as 70% or more, such as 75% or more, such as 80% or more. In a particular embodiment, the host cell is an actinobacterium. The host cell may be selected from the group consisting of Actinomycetales, such as Streptomyces sp., Amycolatopsis sp. or Saccharopolyspora sp. In some embodiments, the host cell is selected from the group consisting of Streptomyces coelicolor, Streptomyces avermitilis, Streptomyces aureofaciens, Streptomyces griseus, Streptomyces parvulus, Streptomyces albus, Streptomyces vinaceus, Streptomyces acrimycinis, Streptomyces calvuligerus, Streptomyces lividans, Streptomyces limosus, Streptomyces rubiqinosis, Streptomyces azureus, Streptomyces glaucenscens, Streptomyces rimosus, Streptomyces violaceoruber, Streptomyces kanamyceticus, Amycolatopsis orientalis, Amycolatopsis mediterranei and Saccharopolyspora erythraea. In a preferred embodiment, the host cell is Streptomyces coelicolor.

In some embodiments, the host cell is from the order Micromonosporales, in particular from the family Micromonosporaceae. In one embodiment, the genus of the host cell is selected from Actinocatenispora, Actinoplanes, Allocatelliglobosispora, Asanoa, Catellatospora, Catelliglobosispora, Catenuloplanes, Couchioplanes, Dactylosporangium, Hamadaea, Jishengella, Krasilnikovia, Longispora, Luedemannella, Micromonospora, Phytohabitans, Phytomonospora, Pilimelia, Planosporangium, Plantactinospora, Polymorphospora, Pseudosporangium, Rhizocola, Rugosimonospora, Salinispora, Solwaraspora, Spirilliplanes, Verrucosispora, Virgisporangium, Wangella or Xiangella.

In some embodiments, the host cell is from the order Streptomycetales, in particular from the family Streptomycetaceae. In one embodiment, the genus of the host cell is selected from Kitasatospora, Parastreptomyces, Streptacidiphilus, Streptomyces or Trichotomospora.

In some embodiments, the host cell is from the order Propionibacteriales, in particular from the family Nocardioidaceae. In one embodiment, the genus of the host cell is selected from Actinopolymorpha, Aeromicrobium, Flindersiella, Friedmanniella, Kribbella, Marmoricola, Micropruina, Mumia, Nocardioides, Pimelobacter, Propionicicella, Propionicimonas, Tenggerimyces or Thermasporomyces.

In some embodiments, the host cell is from the order Propionibacteriales, in particular from the family Propionibacteriaceae. In one embodiment, the genus of the host cell is selected from Aestuariimicrobium, Auraticoccus, Brooklawnia, Granulicoccus, Luteococcus, Mariniluteicoccus, Microlunatus, Naumannella, Ponticoccus, Propionibacterium, Propioniciclava, Propioniferax, Propionimicrobium or Tessaracoccus.

In some embodiments, the host cell is from the order Pseudonocardiales, in particular from the family Pseudonocardiaceae. In one embodiment, the genus of the host cell is selected from Actinoalloteichus, Actinokineospora, Actinomycetospora, Actinophytocola, Actinorectispora, Actinosynnema, Alloactinosynnema, Allokutzneria, Amycolatopsis, Crossiella, Goodfellowiella, Haloechinothrix, Kibdelosporangium, Kutzneria, Labedaea, Lechevalieria, Lentzea, Longimycelium, Prauserella, Prauseria, Pseudonocardia, Saccharomonospora, Saccharopolyspora, Saccharothrix, Saccharothrixopsis, Sciscionella, Streptoalloteichus, Tamaricihabitans, Thermocrispum, Thermotunica, Umezawaea or Yuhushiella.

In some embodiments, the host cell is from the order Streptosporangiales, in particular from the family Nocardiopsaceae. In one embodiment, the genus of the host cell is selected from Allosalinactinospora, Haloactinospora, Marinactinospora, Murinocardiopsis, Nocardiopsis, Salinactinospora, Spinactinospora, Streptomonospora or Thermobifida.

In some embodiments, the host cell is from the order Streptosporangiales, in particular from the family Streptosporangiaceae. In one embodiment, the genus of the host cell is selected from Acrocarpospora, Astrosporangium, Clavisporangium, Herbidospora, Microbispora, Microtetraspora, Nonomuraea, Planobispora, Planomonospora, Planotetraspora, Sinosporangium, Sphaerimonospora, Sphaerisporangium, Streptosporangium, Thermoactinospora, Thermocatellispora or Thermopolyspora.

In some embodiments, the host cell is from the order Streptosporangiales, in particular from the family Thermomonosporaceae. In one embodiment, the genus of the host cell is selected from Actinoallomurus, Actinocorallia, Actinomadura, Spirillospora or Thermomonospora.

The following table lists examples of species for the host cell.

TABLE 1
Non-exhaustive list of suitable host cells.

| Class | Order | Family | Genus | Species |
|---|---|---|---|---|
| Actinobacteria | Micromonosporales | Micromonosporaceae | Actinocatenispora | Actinocatenispora rupis |
| | | | | Actinocatenispora sera |
| | | | | Actinocatenispora thailandica |
| | | | Actinoplanes | Actinoplanes abujensis |
| | | | | Actinoplanes consettensis |
| | | | | Actinoplanes philippinensis |
| | | | Allocatelliglobosispora | Allocatelliglobosispora scoriae |
| | | | Asanoa | Asanoa endophytica |
| | | | | Asanoa ferruginea |
| | | | | Asanoa hainanensis |
| | | | Catellatospora | Catellatospora bangladeshensis |
| | | | | Catellatospora chokoriensis |
| | | | | Catellatospora citrea |
| | | | Catelliglobosispora | Catelliglobosispora koreensis |
| | | | Catenuloplanes | Catenuloplanes atrovinosus |
| | | | | Catenuloplanes castaneus |
| | | | | Catenuloplanes crispus |
| | | | Couchioplanes | Couchioplanes caeruleus |
| | | | Dactylosporangium | Dactylosporangium darangshiense |
| | | | | Dactylosporangium fulvum |
| | | | | Dactylosporangium luridum |
| | | | Hamadaea | Hamadaea flava |
| | | | | Hamadaea tsunoensis |
| | | | Jishengella | Jishengella endophytica |
| | | | Krasilnikovia | Krasilnikovia cinnamomea |
| | | | Longispora | Longispora albida |
| | | | | Longispora fulva |
| | | | Luedemannella | Luedemannella flava |
| | | | | Luedemannella helvata |
| | | | Micromonospora | Micromonospora aquatica |
| | | | | Micromonospora arenae |
| | | | | Micromonospora arenincolae |
| | | | Phytohabitans | Phytohabitans flavus |
| | | | | Phytohabitans houttuyneae |
| | | | | Phytohabitans rumicis |
| | | | Phytomonospora | Phytomonospora endophytica |
| | | | Pilimelia | Pilimelia anulata |
| | | | | Pilimelia columellifera |
| | | | Planosporangium | Planosporangium flavigriseum |
| | | | | Planosporangium mesophilum |
| | | | | Planosporangium thailandense |
| | | | Plantactinospora | Plantactinospora endophytica |
| | | | | Plantactinospora mayteni |
| | | | | Plantactinospora siamensis |
| | | | Polymorphospora | Polymorphospora rubra |
| | | | Pseudosporangium | Pseudosporangium ferrugineum |
| | | | Rhizocola | Rhizocola hellebori |
| | | | Rugosimonospora | Rugosimonospora acidiphila |
| | | | | Rugosimonospora africana |
| | | | Salinispora | alinispora arenicola |
| | | | | Salinispora pacifica |
| | | | | Salinispora tropica |
| | | | Solwaraspora | |
| | | | Spirilliplanes | Spirilliplanes yamanashiensis |
| | | | Verrucosispora | Verrucosispora andamanensis |
| | | | | Verrucosispora fiedleri |
| | | | | Verrucosispora gifhornensis |
| | | | Virgisporangium | Virgisporangium aliadipatigenens |
| | | | | Virgisporangium aurantiacum |
| | | | | Virgisporangium ochraceum |
| | | | Wangella | Wangella harbinensis |
| | | | Xiangella | Xiangella phaseoli |
| | Streptomycetales | Streptomycetaceae | Kitasatospora | Kitasatospora arboriphila |
| | | | | Kitasatospora viridis |
| | | | | Kitasatospora cystarginea |
| | | | Parastreptomyces | Parastreptomyces abscessus |
| | | | Streptacidiphilus | Streptacidiphilus albus |
| | | | | Streptacidiphilus griseus |
| | | | | Streptacidiphilus rugosus |
| | | | | Streptacidiphilus thailandensis |
| | | | | Streptacidiphilus carbonis |
| | | | Streptomyces | Streptomyces albidoflavus group |
| | | | | Streptomyces acrimycinis |
| | | | | Streptomyces avermitilis |
| | | | | Streptomyces aureofaciens |
| | | | | Streptomyces albus |
| | | | | Streptomyces azureus |
| | | | | Streptomyces cattleya |
| | | | | Streptomyces clavuligerus |
| | | | | Streptomyces collinus |
| | | | | Streptomyces eurocidicus |
| | | | | Streptomyces erythrogriseus |
| | | | | Streptomyces filamentosus |
| | | | | Streptomyces fradiae |
| | | | | Streptomyces griseus group |
| | | | | Streptomyces glaucenscens |
| | | | | Streptomyces himastatinicus |
| | | | | Streptomyces hygroscopicus |
| | | | | Streptomyces hygrospinosus |
| | | | | Streptomyces kanamyceticus |
| | | | | Streptomyces lactacystinaeus |
| | | | | Streptomyces lavendulae |
| | | | | Streptomyces levis |
| | | | | Streptomyces libani |
| | | | | Streptomyces limosus |
| | | | | Streptomyces lividans |
| | | | | Streptomyces lomondensis |
| | | | | Streptomyces marinus |
| | | | | Streptomyces melanosporofaciens group |
| | | | | Streptomyces mexicanus |
| | | | | Streptomyces mobaraensis |
| | | | | Streptomyces polyantibioticus |
| | | | | Streptomyces parvulus |
| | | | | Streptomyces purpureus |
| | | | | Streptomyces rapamycinicus |
| | | | | Streptomyces rimosus |
| | | | | Streptomyces rosa |
| | | | | Streptomyces rubiqinosis |
| | | | | Streptomyces scabrisporus |
| | | | | Streptomyces sparsogenes |
| | | | | Streptomyces somaliensis |
| | | | | Streptomyces venezuelae |
| | | | | Streptomyces vinaceus |
| | | | | Streptomyces violaceoruber |
| | | | | Streptomyces viridochromogenes |
| | | | Trichotomospora | Trichotomospora caesia |
| | Propionibacteriales | Nocardioidaceae | Actinopolymorpha | Actinopolymorpha alba |
| | | | | Actinopolymorpha cephalotaxi |
| | | | | Actinopolymorpha pittospori |
| | | | | Actinopolymorpha rutila |
| | | | | Actinopolymorpha singaporensis |
| | | | Aeromicrobium | Aeromicrobium fastidiosum |
| | | | | Aeromicrobium flavum |
| | | | | Aeromicrobium ginsengisoli |
| | | | | Aeromicrobium halocynthiae |
| | | | | Aeromicrobium kazakhstani |
| | | | | Aeromicrobium kwangyangensis |
| | | | | Aeromicrobium marinum |
| | | | Flindersiella | Flindersiella endophytica |
| | | | Friedmanniella | Friedmanniella aerolata |
| | | | | Friedmanniella antarctica |
| | | | | Friedmanniella capsulata |
| | | | | Friedmanniella flava |
| | | | | Friedmanniella lacustris |
| | | | | Friedmanniella lucida |
| | | | | Friedmanniella luteola |
| | | | | Friedmanniella okinawensis |
| | | | | Friedmanniella sagamiharensis |
| | | | | Friedmanniella spumicola |
| | | | Kribbella | Kribbella alba |
| | | | | Kribbella albertanoniae |
| | | | | Kribbella aluminosa |
| | | | | Kribbella amoyensis |
| | | | | Kribbella antibiotica |
| | | | | Kribbella catacumbae |
| | | | | Kribbella flavida |
| | | | Marmoricola | Marmoricola aequoreus |
| | | | | Marmoricola aquaticus |
| | | | | Marmoricola aurantiacus |
| | | | | Marmoricola bigeumensis |
| | | | | Marmoricola ginsengisoli |
| | | | | Marmoricola korecus |
| | | | | Marmoricola pocheonesis |
| | | | | Marmoricola scoriae |
| | | | | Marmoricola soli |
| | | | Micropruina | Micropruina glycogenica |
| | | | Mumia | Mumia flava |
| | | | Nocardioides | Nocardioides aestuarii |
| | | | | Nocardioides agariphilus |
| | | | | Nocardioides albertanoniae |
| | | | | Nocardioides albidus |
| | | | | Nocardioides albus |
| | | | Pimelobacter | Pimelobacter simplex |
| | | | Propionicicella | Propionicicella superfundia |
| | | | Propionicimonas | Propionicimonas paludicola |
| | | | Tenggerimyces | Tenggerimyces flavus |
| | | | | Tenggerimyces mesophilus |
| | | | Thermasporomyces | Thermasporomyces composti |
| | | Propionibacteriaceae | Aestuariimicrobium | Aestuariimicrobium kwangyangense |
| | | | Auraticoccus | Auraticoccus monumenti |
| | | | Brooklawnia | Brooklawnia cerclae |
| | | | | Brooklawnia massiliensis |
| | | | Granulicoccus | Granulicoccus phenolivorans |
| | | | Luteococcus | Granulicoccus phenolivorans |
| | | | | Luteococcus peritonei |
| | | | | Luteococcus sanguinis |
| | | | | Luteococcus sediminum |
| | | | Mariniluteicoccus | Mariniluteicoccus endophyticus |
| | | | | Mariniluteicoccus flavus |
| | | | Microlunatus | Microlunatus aurantiacus |
| | | | | Microlunatus endophyticus |
| | | | | Microlunatus ginsengisoli |
| | | | | Microlunatus ginsengiterrae |
| | | | | Microlunatus panaciterrae |
| | | | | Microlunatus parietis |
| | | | Naumannella | Naumannella halotolerans |
| | | | Ponticoccus | Ponticoccus gilvus |
| | | | Propionibacterium | Propionibacterium acidifaciens |
| | | | | Propionibacterium acidipropionici |
| | | | | ropionibacterium acnes |
| | | | | Propionibacterium avidum |
| | | | Propioniciclava | Propioniciclava tarda |
| | | | Propioniferax | Propioniferax innocua |
| | | | Propionimicrobium | Propionimicrobium lymphophilum |
| | | | Tessaracoccus | Tessaracoccus bendigoensis |
| | | | | Tessaracoccus flavescens |
| | | | | Tessaracoccus flavus |
| | | | | Tessaracoccus lapidicaptus |
| | | | | Tessaracoccus lubricantis |
| | | | | Tessaracoccus oleiagri |
| | | | | Tessaracoccus profundi |
| | | | | Tessaracoccus rhinocerotis |
| | Pseudonocardiales | Pseudonocardiaceae | Actinoalloteichus | Actinoalloteichus alkalophilus |
| | | | | Actinoalloteichus cyanogriseus |
| | | | Actinokineospora | Actinokineospora auranticolor |
| | | | | Actinokineospora baliensis |
| | | | | Actinokineospora bangkokensis |
| | | | | Actinokineospora cianjurensis |
| | | | | Actinokineospora cibodasensis |
| | | | | Actinokineospora diospyrosa |
| | | | | Actinokineospora enzanensis |
| | | | | Actinokineospora inagensis |
| | | | Actinomycetospora | Actinomycetospora chiangmaiensis |
| | | | | Actinomycetospora chibensis |
| | | | | Actinomycetospora chlora |
| | | | | Actinomycetospora cinnamomea |
| | | | Actinophytocola | Actinophytocola burenkhanensis |
| | | | | Actinophytocola corallina |
| | | | | Actinophytocola gilvus |
| | | | | Actinophytocola oryzae |
| | | | | Actinophytocola sediminis |
| | | | | Actinophytocola timorensis |
| | | | | Actinophytocola xinjiangensis |
| | | | Actinorectispora | Actinorectispora indica |
| | | | Actinosynnema | Actinosynnema mirum |
| | | | Alloactinosynnema | Alloactinosynnema album |
| | | | | Alloactinosynnema iranicum |
| | | | Allokutzneria | Allokutzneria albata |
| | | | | Allokutzneria multivorans |
| | | | | Allokutzneria oryzae |
| | | | Amycolatopsis | Amycolatopsis alba |
| | | | | Amycolatopsis azurea |
| | | | | Amycolatopsis coloradensis |
| | | | | Amycolatopsis coloradensis |
| | | | | Amycolatopsis halophila |
| | | | | Amycolatopsis lurida |
| | | | | Amycolatopsis mediterranei |
| | | | | Amycolatopsis pigmentata |
| | | | | Amycolatopsis taiwanensis |
| | | | Crossiella | Crossiella cryophila |
| | | | | Crossiella equi |
| | | | Goodfellowiella | Goodfellowiella coeruleoviolacea |
| | | | Haloechinothrix | Haloechinothrix alba |
| | | | Kibdelosporangium | Haloechinothrix alba |
| | | | Kutzneria | Kutzneria albida |
| | | | Labedaea | Labedaea rhizosphaerae |
| | | | Lechevalieria | Lechevalieria aerocolonigenes |
| | | | | Lechevalieria atacamensis |
| | | | | Lechevalieria deserti |
| | | | | Lechevalieria flava |
| | | | | Lechevalieria fradiae |
| | | | | Lechevalieria nigeriaca |
| | | | | Lechevalieria roselyniae |
| | | | | Lechevalieria xinjiangensis |
| | | | Lentzea | Lentzea albida |
| | | | | Lentzea albidocapillata |
| | | | | Lentzea californiensis |
| | | | | Lentzea flaviverrucosa |
| | | | | Lentzea jiangxiensis |
| | | | | Lentzea kentuckyensis |
| | | | | Lentzea violacea |
| | | | | Lentzea waywayandensis |
| | | | Longimycelium | Longimycelium tulufanense |
| | | | Prauserella | Prauserella aidingensis |
| | | | | Prauserella alba |
| | | | | Prauserella coralliicola |
| | | | | Prauserella flava |
| | | | Prauseria | Prauseria hordei |
| | | | Pseudonocardia | Pseudonocardia acaciae |
| | | | | Pseudonocardia asaccharolytica |
| | | | | Pseudonocardia spinosispora |
| | | | | Pseudonocardia sulfidoxydans |
| | | | | Pseudonocardia tetrahydrofuranoxydans |
| | | | | Pseudonocardia tetrahydrofuranoxydans |
| | | | Saccharomonospora | Saccharomonospora azurea |
| | | | | Saccharomonospora cyanea |
| | | | | Saccharomonospora viridis |
| | | | | Saccharomonospora marina |
| | | | Saccharopolyspora | Saccharopolyspora antimicrobica |
| | | | | Saccharopolyspora cavernae |
| | | | | Saccharopolyspora cebuensis |
| | | | | Saccharopolyspora dendranthemae |
| | | | | Saccharopolyspora emeiensis |
| | | | | Saccharopolyspora endophytica |
| | | | | Saccharopolyspora erythraea |
| | | | | Saccharopolyspora spinosa |
| | | | | Saccharopolyspora rosea |
| | | | Saccharothrix | Lentzea flavoverrucoides |
| | | | | Saccharothrix algeriensis |
| | | | | Saccharothrix australiensis |
| | | | | Saccharothrix carnea |
| | | | | Saccharothrix coeruleofusca |
| | | | | Saccharothrix espanaensis |
| | | | Saccharothrixopsis | Saccharothrixopsis albidus |
| | | | Sciscionella | Sciscionella marina |
| | | | Streptoalloteichus | Streptoalloteichus hindustanus |
| | | | | Streptoalloteichus tenebrarius |
| | | | Tamaricihabitans | Tamaricihabitans halophyticus |
| | | | Thermocrispum | Thermocrispum agreste |
| | | | | Thermocrispum municipale |
| | | | Thermotunica | Thermotunica guangxiensis |
| | | | Umezawaea | Umezawaea tangerina |
| | | | Yuhushiella | Yuhushiella deserti |
| | Streptosporangiales | Nocardiopsaceae | Allosalinactinospora | Allosalinactinospora lopnorensis |
| | | | Haloactinospora | Haloactinospora alba |
| | | | Marinactinospora | Marinactinospora thermotolerans |
| | | | Murinocardiopsis | Murinocardiopsis flavida |
| | | | Nocardiopsis | Nocardiopsis aegyptia |
| | | | | Nocardiopsis alba |
| | | | | Nocardiopsis algeriensis |
| | | | | Nocardiopsis alkaliphila |
| | | | | Nocardiopsis baichengensis |
| | | | | Nocardiopsis chromatogenes |
| | | | | Nocardiopsis ganjiahuensis |
| | | | | Nocardiopsis lucentensis |
| | | | | Nocardiopsis potens |
| | | | | Nocardiopsis synnemataformans |
| | | | | Nocardiopsis prasina |
| | | | | Nocardiopsis halophila |
| | | | Salinactinospora | Salinactinospora qingdaonensis |
| | | | | Salinactinospora qingdaonensis |
| | | | Spinactinospora | Streptomonospora alba |
| | | | Streptomonospora | Streptomonospora algeriensis |
| | | | | Streptomonospora amylolytica |
| | | | | Streptomonospora arabica |
| | | | | Streptomonospora flavalba |
| | | | | Streptomonospora halophila |
| | | | | Streptomonospora nanhaiensis |
| | | | | Streptomonospora salina |
| | | | | Streptomonospora sediminis |
| | | | Thermobifida | Thermobifida cellulosilytica |
| | | | | Thermobifida fusca |
| | | | | Thermobifida alba |
| | | Streptosporangiaceae | Acrocarpospora | Acrocarpospora corrugata |
| | | | | Acrocarpospora macrocephala |
| | | | | Acrocarpospora phusangensis |
| | | | | Acrocarpospora pleiomorpha |
| | | | Astrosporangium | Astrosporangium hypotensionis |
| | | | Clavisporangium | Clavisporangium rectum |
| | | | Herbidospora | Herbidospora cretacea |
| | | | | Herbidospora daliensis |
| | | | | Herbidospora mongoliensis |
| | | | | Herbidospora sakaeratensis |
| | | | | Herbidospora yilanensis |
| | | | Microbispora | Microbispora amethystogenes |
| | | | | Microbispora bryophytorum |
| | | | | Microbispora camponoti |
| | | | | Microbispora corallina |
| | | | | Microbispora griseoalba |
| | | | | Microbispora hainanensis |
| | | | | Microbispora mesophila |
| | | | | Microbispora rosea |
| | | | Microtetraspora | Microtetraspora fusca |
| | | | | Microtetraspora glauca |
| | | | | Microtetraspora malaysiensis |
| | | | | Microtetraspora niveoalba |
| | | | Nonomuraea | Nonomuraea aegyptia |
| | | | | Nonomuraea africana |
| | | | | Nonomuraea angiospora |
| | | | | Nonomuraea antimicrobica |
| | | | | Nonomuraea asiatica |
| | | | | Nonomuraea aurea |
| | | | | Nonomuraea bangladeshensis |
| | | | | Nonomuraea candida |
| | | | Planobispora | Planobispora longispora |
| | | | | Planobispora rosea |
| | | | | Planobispora siamensis |
| | | | | Planobispora takensis |
| | | | Planomonospora | Planomonospora alba |
| | | | | Planomonospora parontospora |
| | | | Planotetraspora | Planotetraspora kaengkrachanensis |
| | | | | Planotetraspora mira |
| | | | | Planotetraspora phitsanulokensis |
| | | | | Planotetraspora silvatica |
| | | | | Planotetraspora thailandica |
| | | | Sinosporangium | Sinosporangium album |
| | | | | Sinosporangium siamense |
| | | | Sphaerimonospora | Sphaerimonospora cavernae |
| | | | Sphaerisporangium | Sphaerisporangium album |
| | | | | Sphaerisporangium cinnabarinum |
| | | | | Sphaerisporangium flaviroseum |
| | | | Streptosporangium | Sphaerisporangium album |
| | | | | Sphaerisporangium cinnabarinum |
| | | | | Sphaerisporangium flaviroseum |
| | | | | Sphaerisporangium krabiense |
| | | | | Sphaerisporangium melleum |
| | | | | Sphaerisporangium rubeum |
| | | | | Sphaerisporangium rufum |
| | | | | Sphaerisporangium siamense |
| | | | | Sphaerisporangium viridialbum |
| | | | Thermoactinospora | Thermoactinospora rubra |
| | | | Thermocatellispora | Thermocatellispora tengchongensis |
| | | | Thermopolyspora | Thermopolyspora flexuosa |
| | | Thermomonosporaceae | Actinoallomurus | Actinoallomurus caesius |
| | | | | Actinoallomurus coprocola |
| | | | | Actinoallomurus fulvus |
| | | | | Actinoallomurus iriomotensis |
| | | | | Actinoallomurus acaciae |
| | | | | Actinoallomurus acanthiterrae |
| | | | | Actinoallomurus amamiensis |
| | | | | Actinoallomurus bryophytorum |
| | | | Actinocorallia | Actinocorallia aurantiaca |
| | | | | Actinocorallia aurea |
| | | | | Actinocorallia cavernae |
| | | | | Actinocorallia glomerata |
| | | | | Actinocorallia herbida |
| | | | | Actinocorallia libanotica |
| | | | | Actinocorallia longicatena |
| | | | | Actinocorallia spatholoba |
| | | | Actinomadura | Actinomadura alba |
| | | | | Actinomadura amylolytica |
| | | | | Actinomadura apis |
| | | | | Actinomadura atramentaria |
| | | | | Actinomadura bangladeshensis |
| | | | | Actinomadura catellatispora |
| | | | | Actinomadura cellulosilytica |
| | | | | Actinomadura chibensis |
| | | | Spirillospora | Spirillospora albida |
| | | | | Spirillospora rubra |
| | | | Thermomonospora | Thermomonospora curvata |
| | | | | Thermomonospora chromogena |

Method for Generating Random-Sized Deletions or Indels Around a Target Site

In a first aspect, the invention relates to a method for generating at least one deletion around at least one target nucleic acid sequence comprised within a host cell having a non-homologous end-joining (NHEJ) pathway which is at least partly deficient,
said method comprising the steps of:
(i) optionally, restoring the full functionality of the NHEJ pathway,
(ii) inducing a CRISPR-Cas9 system in said host cell, wherein said CRISPR-Cas9 system is able to generate at least one break in said at least one target nucleic acid sequence and wherein the CRISPR-Cas9 system comprises a Cas9 nuclease and at least one guiding means,
thereby generating:
a. if the method does not comprise step (i), at least one random-sized deletion around said at least one target nucleic acid sequence, wherein said at least one deletion is a random-sized deletion of at least 1 bp; or
b. if the method does comprise step (i), at least one indel around said at least one target nucleic acid sequence, wherein said at least one indel is a deletion or insertion of at least1 bp.

The methods the present disclosure thus take advantage of the fact that in host cells, wherein the NHEJ pathway is at least partly deficient, a CRISPR-Cas9 system can be induced and generates either random-sized deletions around a target site, or indels around a target site if the functionality of the NHEJ pathway is restored prior to or simultaneously with induction of the CRISPR-Cas9 system.

Method for Generating Random-Sized Deletions Around a Target Site

In some embodiments, the method does not comprise step (i). In other words, the NHEJ pathway is maintained partly deficient. The present disclosure thus provides a method for generating at least one random-sized deletion around at least one target nucleic acid sequence comprised within a host cell having a non-homologous end-joining (NHEJ) pathway which is at least partly deficient, said method comprising the step of inducing a CRISPR-Cas9 system in a host cell, said CRISPR-Cas9 system being able to generate at least one break in said at least one target nucleic acid sequence, thereby generating at least one deletion around said at least one target nucleic acid sequence, wherein said at least one deletion is a deletion of at least 1 bp.

The method is based on the surprising finding that performing CRISPR-Cas9 directed gene editing in organisms having a partly deficient NHEJ pathway leads to the generation of random-sized deletions around a target nucleic acid sequence. This is surprising because performing CRISPR-Cas9 directed editing in organisms lacking NHEJ was believed to be lethal (Citorik, R. J. et, al 2014, Gomaa, A. et, al 2014, Bikard, D., et, al, 2014). The gene editing is preferably performed without homology arms so that the repair of the at least one break generated by Cas9 is directed towards the NHEJ pathway. Thus in some embodiments, the method for generating at least one deletion described herein is performed with the proviso that the editing is not done with a homologous template.

In some embodiments, the guiding means comprises at least one sgRNA and/or at least one crRNA/tracrRNA set.

Also disclosed herein is a method for generating at least one deletion around at least one target nucleic acid sequence comprised within a host cell having a non-homologous end-joining (NHEJ) pathway which is at least partly deficient, said method comprising the step of inducing a CRISPR-Cas9 system in a host cell, said CRISPR-Cas9 system being able to generate at least one break in said at least one target nucleic acid sequence, thereby generating at least one deletion around said at least one target nucleic acid sequence, wherein said at least one deletion is a deletion of at least 1 bp, wherein the CRISPR-Cas9 system comprises a Cas9 nuclease encoded by a polynucleotide having at least 93% identity with SEQ ID NO: 1, such as at least 94% identity, such as at least 95% identity, such as at least 96% identity, such as at least 97% identity, such as at least 98% identity, such as at least 99% identity, such as 100% identity with SEQ ID NO: 1. In some embodiments, the Cas9 nuclease is identical to SEQ ID NO: 2.

NHEJ

The method disclosed herein for generating random-sized deletions around at least one target nucleic acid sequence is preferably performed in a host cell wherein the NHEJ pathway is at least partly deficient.

The NHEJ pathway involves four activities dependent on two groups of proteins:
(a) the Ku proteins, which bind to DNA double-strand break ends and are required for the non-homologous end joining;
(b) the ligase, such as the ligase D ligD, which can perform the activities of ligase, polymerase and primase.

In some embodiments, the NHEJ pathway of the host cell thus lacks at least one of the four NHEJ activities defined as:
a DNA-binding activity,
a primase activity,
a ligase activity,
a polymerase activity.

The DNA-binding activity is typically performed by Ku proteins such as Ku70, Ku80, or homologues, orthologues or paralogues thereof. The primase activity can be performed by a eukaryotic-archeal DNA primase (EP) or a homologue, an orthologue or a paralogue thereof, or by a ligase D or a homologue, an orthologue or a paralogue thereof. The ligase activity is typically performed by ligase D or a homologue, an orthologue or a paralogue thereof. The polymerase activity is typically performed by a ligase D or a homologue, an orthologue or a paralogue thereof.

As understood herein, a functional NHEJ pathway comprises all four activities, e.g. it may comprise one Ku protein with a DNA-binding activity and a ligase capable of performing the activities of ligase, polymerase and primase. In some embodiments, the activities of ligase, polymerase and primase are performed by the same or by two, three or four different proteins, peptides or domains. A partly deficient NHEJ pathway lacks at least one of the four activities. In some embodiments, the NHEJ pathway of the host cell thus lacks at least one of the DNA-binding activity, of the ligase activity, of the polymerase activity and of the primase activity. In a preferred embodiment, the NHEJ pathway is partly deficient because the ligase can only perform the primase activity. For example, the Ku proteins are present and functional, but the ligase lacks the ligase activity.

The NHEJ pathway may be deficient because it is naturally deficient in the host cell, or because at least one of the four activities has been inactivated. In some embodiments, the DNA-binding activity is inactivated, e.g. by targeted deletion of the nucleic acid sequence(s) encoding the Ku protein(s). In further embodiments, the primase activity is inactivated. In other embodiments, the ligase activity is inactivated. In yet other embodiments, the polymerase activity is inactivated. Preferably, at least the ligase activity is inactivated. Other methods for inactivating at least one of the four NHEJ activities are known to the skilled person.

Host cells where the NHEJ pathway is naturally deficient can be identified by methods known in the art, such as gene mining or sequence blasting.

The activities referred to above may be performed by a domain, peptide or protein. The nucleic acid sequences encoding the domain, peptide or protein capable of performing said activities may be comprised within the genome of the host cell or may be comprised on a vector.

Target Nucleic Acid

The method disclosed herein is particularly useful for generating random-sized deletions around at least one target nucleic acid sequence of interest. The present method can thus be used in order to generate clonal libraries containing a plurality of cells having deletions of different sizes around at least one target nucleic acid of interest, as described below. The method can thus be useful for, but not limited to, the investigation of pathway regulations and identification of metabolite production bottlenecks, the screening of producer strains and the identification of new compounds produced by the host cell. The libraries thus generated are not completely random in that the target nucleic acid is predefined.

The target nucleic acid sequence may be comprised within any nucleic acid sequence of interest. For example, the target sequence may be comprised within or may comprise an open reading frame or a putative open reading frame, or it may be comprised within or may comprise a regulatory region or a putative regulatory region, such as an enhancer, a promoter, an insulator, a terminator.

The target nucleic acid sequence may be involved in a pathway of interest. In some embodiments, the target nucleic acid encodes an enzyme or a protein. In other embodiments, the target nucleic acid is comprised within or comprises a biosynthetic gene or a putative biosynthetic gene. In some embodiments, the biosynthetic gene is involved in the synthesis of a secondary metabolite.

In some embodiments, the target nucleic acid sequence is comprised within a gene cluster. In specific embodiments, the gene cluster is a secondary metabolite gene cluster.

There is thus disclosed herein a method for editing a target nucleic acid sequence optionally comprised within or comprising a gene cluster, where the target nucleic acid sequence is involved or is suspected of being involved in the biosynthesis of a secondary metabolite.

In some embodiments, the secondary metabolite is selected from the group consisting of antibiotics, herbicides, anti-cancer agents, immunosuppressants, flavors, parasiticides and proteins. The term ‘parasiticide’ is to be understood in its broadest sense as an agent capable of inactivating or killing any undesirable organism and thus comprises insecticides, anthelmintic compounds, larvacides, antiparasitic agents and antiprotozoal agents.

In some embodiments, the secondary metabolite is an antibiotic selected from the group consisting of apramycin, bacitracin, chloramphenicol cephalosporins, cycloserine, erythromycin, fosfomycin, gentamicin, kanamycin, kirromycin, lassomycin, lincomycin, lysolipin, microbisporicin, neomycin, noviobiocin, nystatin, nitrofurantoin, platensimycin, pristinamycins, rifamycin, streptomycin, teicoplanin, tetracycline, tinidazole, ribostamycin, daptomycin, vancomycin, viomycin and virginiamycin.

In other embodiments, the secondary metabolite is a herbicide selected from the group consisting of bialaphos, resormycin and phosphinothricin.

In yet other embodiments, the secondary metabolite is an anti-cancer agent selected from the group consisting of doxorubicin, salinosporamides, aclarubicin, pentostatin, peplomycin, thrazarine and neocarcinostatin.

In yet other embodiments, the secondary metabolite is an immunosuppressant selected from the group consisting of rapamycin, FK520, FK506, cyclosporine, ushikulides, pentalenolactone I and hygromycin A.

In yet other embodiments, the secondary metabolite is a flavor such as geosmin.

In yet other embodiments, the secondary metabolite is a parasiticide such as an insecticide, an anthelmintic, a larvacide, or an antiprotozoal agent such as spinsad or avermectin.

In other embodiments, the target nucleic acid codes for an enzyme selected from the group consisting of an amylase, a protease, a cellulase, a chitinase, a keratinase and a xylanase.

In some embodiments, only one target nucleic acid sequence is targeted for editing and generation of random-sized deletions. In other embodiments, more than one target nucleic acid sequence is targeted and the method is a multiplex method. Thus the method can be used for generating at least one deletion around at least one target nucleic acid sequence, such as at least two deletions around at least two target nucleic acid sequences, such as at least three deletions around at least three target nucleic acid sequences, such as at least four deletions around at least four target nucleic acid sequences, such as at least five deletions around at least five target nucleic acid sequences, or more, wherein each deletion as a deletion of at least 1 bp. The method can thus be used for generating one deletion around one target nucleic acid sequence, or two deletions around at least two target nucleic acid sequences, or three deletions around three target nucleic acid sequences, or four deletions around four target nucleic acid sequences, or five deletions around five target nucleic acid sequences, or more. As explained above, in the case of multiplex editing, a guiding means is preferably provided for each target nucleic acid sequence.

In some embodiments, the at least one deletion results in the inactivation of at least one gene. In some embodiments, the at least one gene is comprised within a gene cluster. In other embodiments, the at least one gene is not comprised within a gene cluster.

The at least one deletion generated by the present method is a deletion of at least 1 bp and may range over several thousands kilobases. In some embodiments, the deletion is a deletion of 1 to 2. 10⁶ bp, such as 1 to 1. 10⁶ bp, such as 1 to 500000 bp, such as 1 to 400000 bp, such as 1 to 300000 bp, such as 1 to 200000 bp, such as 1 to 100000 bp, such as 2 to 75000 bp, such as 3 to 50000 bp, such as 4 to 40000 bp, such as 5 to 30000 bp, such as 10 to 20000 bp, such as 25 to 10000 bp, such as 50 to 9000 bp, such as 75 to 8000 bp, such as 100 to 7000 bp, such as 150 to 6000 bp, such as 200 to 5000 bp, such as 250 to 4000 bp, such as 300 to 3000 bp, such as 400 to 2000 bp, such as 500 to 1000 bp, such as 600 to 900 bp, such as 700 to 800 bp. In some embodiments, the deletion is a deletion of at least 1 bp, such as at least 2 bp, such as at least 3 bp, such as at least 4 bp, such as at least 5 bp, such as at least 10 bp, such as at least 15 bp, such as at least 20 bp, such as at least 50 bp, such as at least 100 bp, such as at least 250 bp, such as at least 500 bp. In some embodiments, the deletion is a deletion of 1 to 100 bp, such as 1 to 75 bp, such as 1 to 50 bp, such as 1 to 40 bp, such as 1 to 30 bp, such as 1 to 20 bp, such as 1 to 10 bp, such as 1 to 9 bp, such as 1 to 8 bp, such as 1 to 7 bp, such as 1 to 6 bp, such as 1 to 5 bp, such as 1 to 4 bp, such as 1 to 3 bp, such as 1 to 2 bp.

Efficiency and Off-Target Effects

Several parameters can have an impact on the efficiency of the present method for generating random-sized deletions around at least one target sequence. Some parameters can be adjusted as known in the art. Parameters susceptible of having an impact on the efficiency include, but are not limited to: the sequence of the guiding means (sgRNA or crRNA/tracrRNA), the sequence of the target nucleic acid, the GC content of the host cell and the GC content of the target nucleic acid sequence.

The method can be performed with relatively few off-target effects. In some embodiments, the desired deletion is generated in more than 1% of the host cells, such as in more than 5% of the host cells, such as in more than 10% of the host cells, such as in more than 15% of the host cells, such as in more than 20% of the host cells, such as in more than 25% of the host cells, such as in more than 30% of the host cells, such as in more than 35% of the host cells, such as in more than 40% of the host cells, such as in more than 45% of the host cells, such as in more than 50% of the host cells, such as in more than 55% of the host cells, such as in more than 60% of the host cells, such as in more than 65% of the host cells, such as in more than 70% of the host cells, such as in more than 75% of the host cells, such as in more than 80% of the host cells, such as in more than 85% of the host cells, such as in more than 90% of the host cells, such as in more than 95% of the host cells, such as in 100% of the host cells.

Characterisation and Screening

The present method can thus be used for generating random sized deletions around a target nucleic acid sequence of interest, for example a sequence encoding for a gene involved in a pathway of interest. This can result in a plurality of clones having random-sized deletions around the target sequence. These clones can then be further analysed or screened. For example, producer strains having advantageous production profiles for a desired compound can be selected.

In some embodiments, it may be of interest to determine the size of the at least one deletion for a particular clone. Thus the method may comprise a further step of determining the size of the at least one deletion. Methods for determining the size of a deletion are known in the art and include, but are not limited to, whole genome sequencing, pulsed field gel electrophoresis, nucleic acid amplification-based methods such as PCR, for example followed by restriction analysis and detection of the PCR products on a gel and determination of the size of the products using an appropriate marker. The PCR products can also be sequenced if precise determination of the size of the deletion is desired.

In some embodiments, the method further comprises a step of selection of clones having the desired characteristics. Such selection methods are known in the art and encompass screening methods, chemical analysis of the related gene products (proteins or metabolites), sequencing of the related gene regions, and/or analysis of the gene expression level.

Clonal Library

In one aspect, the disclosure relates to a clonal library obtainable by the method for generating random-sized deletions around at least one target nucleic acid sequence as described herein above. Such clonal libraries comprise a plurality of clones obtained by said method, wherein each clone harbours at least one deletion around at least one target nucleic acid sequence, wherein each of said deletions is a deletion of at least 1 bp.

The clonal libraries may be generated by multiplex methods, wherein more than one deletion is generated around more than one target nucleic acid in each clone.

The clonal libraries may be libraries of archaea, prokaryotes or eukaryotes. In one embodiment, the clonal library is a prokaryotic clonal library. In some embodiments, the clones of the clonal library have a high GC content. In some embodiments, the GC content is higher than 45%, such as 50% or more, such as 55% or more, such as 60% or more, such as 65% or more, such as 70% or more, such as 75% or more, such as 80% or more. In a particular embodiment, the clonal library is a library of an actinobacterium, for example selected from the group consisting of Actinomycetales, such as Streptomyces sp., Amycolatopsis sp. or Saccharopolyspora sp. In some embodiments, the clonal library is a library of clones derived from Streptomyces coelicolor, Streptomyces avermitilis, Streptomyces aureofaciens, Streptomyces griseus, Streptomyces parvulus, Streptomyces albus, Streptomyces vinaceus, Streptomyces acrimycinis, Streptomyces calvuligerus, Streptomyces lividans, Streptomyces limosus, Streptomyces rubiqinosis, Streptomyces azureus, Streptomyces glaucenscens, Streptomyces rimosus, Streptomyces violaceoruber, Streptomyces kanamyceticus, Amycolatopsis orientalis, Amycolatopsis mediterranei or Saccharopolyspora erythraea. In a preferred embodiment, the clonal library is a library of Streptomyces coelicolor clones.

Method for Generating Precise Indels Around a Target Site

In some embodiments, the method comprises the step of restoring full functionality of the at least partly deficient NHEJ pathway in the host cell prior to or simultaneously with the step of inducing a CRISPR-Cas9 system. This results in generation of at least one indel around at least one target nucleic acid sequence comprised within a host cell having a non-homologous end-joining (NHEJ) pathway which is at least partly deficient, said method comprising the steps of (i) restoring the full functionality of the NHEJ pathway in said host cell; (ii) inducing a CRISPR-Cas9 system in said host cell, said CRISPR-Cas9 system being able to generate at least one break in said at least one target nucleic acid sequence, thereby generating at least one indel around said at least one target nucleic acid sequence, wherein said at least one indel is an insertion or a deletion of at least 1 bp such as at least 2 bp, such as at least 3 bp, such as at least 4 bp, such as at least 5 bp, such as at least 10 bp, such as at least 15 bp, such as at least 20 bp, such as at least 50 bp, such as at least 100 bp, such as at least 250 bp, such as at least 500 bp.

In some embodiments, the guiding means comprises at least one sgRNA and/or at least one crRNA/tracrRNA set.

In a host cell having a partly deficient NHEJ pathway, CRISPR-Cas9 gene editing results in the generation of random-sized deletions around the target sites, as disclosed in the first aspect of the invention. The deletions can, as described above and as shown in the examples, be very large. While this may be of interest in some cases, it may sometimes be desirable to generate precise deletions or insertions around target sequences instead. The terms ‘precise deletion’ or ‘precise insertion’ or ‘precise indel’ preferably refer herein to to insertions, deletions or indels of which the size can be determined in advance, as opposed to random-sized deletions. These can be short deletions, insertions or indels, i.e. spanning over small areas as detailed below. The second aspect of the invention describes how this can be achieved. In some embodiments, the gene editing is performed without homology arms so that the repair of the at least one break generated by Cas9 is directed towards the NHEJ pathway. In other embodiments, the gene editing is performed with homology arms so that the repair of the at least one break generated by Cas9 is directed toward the HDR pathway.

There is disclosed herein a method for generating at least one indel around at least one target nucleic acid sequence comprised within a host cell having a non-homologous end-joining (NHEJ) pathway which is at least partly deficient, said method comprising the steps of (i) restoring the full functionality of the NHEJ pathway in said host cell; (ii) inducing a CRISPR-Cas9 system in said host cell, said CRISPR-Cas9 system being able to generate at least one break in said at least one target nucleic acid sequence, thereby generating at least one indel around said at least one target nucleic acid sequence, wherein said at least one indel is an indel of at least 1 bp, wherein the CRISPR-Cas9 system comprises a Cas9 nuclease encoded by a polynucleotide having at least 93% identity with SEQ ID NO: 1, such as at least 94% identity, such as at least 95% identity, such as at least 96% identity, such as at least 97% identity, such as at least 98% identity, such as at least 99% identity, such as 100% identity with SEQ ID NO: 1. In some embodiments, the Cas9 nuclease is identical to SEQ ID NO: 2.

Restoring NHEJ

The method disclosed herein for generating precise indels around at least one target nucleic acid sequence is preferably performed in a host cell wherein the NHEJ pathway is at least partly deficient.

Host cells where the NHEJ pathway is naturally deficient can be identified by methods known in the art, such as gene mining or sequence blasting.

The NHEJ pathway involves four activities dependent on two groups of proteins:
(a) the Ku proteins, which bind to DNA double-strand break ends and are required for the non-homologous end joining;
(b) the ligase, such as the ligase D ligD, which can perform the activities of ligase, polymerase and primase.

In some embodiments, the NHEJ pathway of the host cell thus lacks at least one of four activities defined as:
a DNA-binding activity,
a primase activity,
a ligase activity
a polymerase activity.

The DNA-binding activity is typically performed by Ku proteins such as Ku70, Ku80, or homologues, orthologues or paralogues thereof. The primase activity can be performed by a eukaryotic-archeal DNA primase (EP) or a homologue, an orthologue or a paralogue thereof, or by a ligase D or a homologue, an orthologue or a paralogue thereof. The ligase activity is typically performed ligase D or a homologue, an orthologue or a paralogue thereof. The polymerase activity is typically performed by a ligase D or a homologue, an orthologue or a paralogue thereof.

As understood herein, a functional NHEJ pathway comprises all four activities, e.g. it comprises one Ku protein with a DNA-binding activity and a ligase capable of performing the activities of ligase and primase. A partly deficient NHEJ pathway lacks at least one of the four activities. In some embodiments, the NHEJ pathway of the host cell thus lacks at least one of the DNA-binding activity, of the polymerase activity, of the ligase activity and of the primase activity. In a preferred embodiment, the NHEJ pathway is partly deficient because the ligase can only perform the primase activity. For example, the Ku proteins are present and functional, but the ligase lacks the ligase activity.

The NHEJ pathway may be deficient because it is naturally deficient in the host cell, or because at least one of the four activities has been inactivated. In some embodiments, the DNA-binding activity is inactivated, e.g. by targeted deletion of the nucleic acid sequence(s) encoding the Ku protein(s). In further embodiments, the primase activity is inactivated. In other embodiments, the ligase activity is inactivated. In yet other embodiments, the polymerase activity is inactivated. Preferably, at least the ligase activity is inactivated. Other methods for inactivating at least one of the four NHEJ activities are known to the skilled person.

The activities referred to above may be performed by a domain, peptide or protein. The nucleic acid sequences encoding the domain, peptide or protein capable of performing said activities may be comprised within the genome of the host cell or may be comprised on a vector.

In order to generate precise indels around at least one target nucleic acid sequence, the at least one NEHJ activity which is lacking in the host cell may need to be restored. This can be achieved by introducing a nucleic acid sequence comprising a sequence encoding a domain, a peptide or a protein capable of performing said lacking NHEJ activity into the host cell.

The nucleic acid sequence comprising a sequence such as an open reading frame encoding said domain, peptide or protein capable of performing said lacking activity (hereinafter also referred to as ‘the nucleic acid sequence encoding said lacking activity’) can be introduced into the host cell's genome, e.g. on a chromosome, or it can be comprised within a vector and the vector can be introduced within the host cell.

The nucleic acid sequence encoding the lacking NHEJ activity can be under the control of an inducible promoter and may comprise other elements besides an open reading frame encoding the activity. For example, the nucleic acid sequence may further comprise a terminator, a sequence encoding a selection marker and/or a sequence encoding a fluorescent protein.

In some embodiments, the nucleic acid sequence encoding the lacking NHEJ activity and the nucleic acid sequence encoding Cas9 may be comprised within a single nucleic acid, for example they may be on the same vector or they may be integrated at the same location in the genome of the host cell. Likewise, the nucleic acid sequence encoding the lacking NHEJ activity and the nucleic acid sequence encoding the guiding means may be comprised within a single nucleic acid, for example they may be on the same vector or they may be integrated at the same location in the genome of the host cell. In some embodiments, the nucleic acid sequence encoding the lacking NHEJ activity, the nucleic acid sequence encoding Cas9 and the nucleic acid sequence encoding the guiding means are all comprised within a single nucleic acid. Each of these three elements may also be comprised each within one nucleic acid.

In some embodiments, the host cell is lacking more than one NHEJ activity. It may lack two NHEJ activities or it may lack three NHEJ activities or four NHEJ activities. In order to restore NHEJ, it may be necessary to restore each of the lacking activities. The nucleic acid sequences encoding each of the lacking activities can be comprised within a single nucleic acid, or they can be comprised within different nucleic acids. The guiding means and Cas9 may be comprised within the same nucleic acid as one or all of the sequences encoding the lacking activity, or they may be comprised within a different nucleic acid, as above.

In some embodiments, restoration of the lacking NHEJ activity or activities is achieved by introduction of a heterologous gene encoding a domain, protein or peptide capable of performing the lacking activity when it is expressed in the host cell. Suitable heterologous genes can be identified by methods such as blasting a genome database using a nucleic acid sequence encoding the lacking activity as a query. The query sequence is preferably the sequence of a cell naturally possessing the activity lacking in the host cell in which the method is to be performed. Preferably, the query sequence is taken from a cell which is related to the host cell, for example from a cell which is phylogenetically close to the host cell.

In embodiments where the host cell having a partly deficient NHEJ pathway is an actinobacterium, the cell from which the query sequence is derived is preferably also an actinobacterium.

Once a sequence encoding the lacking activity has been identified, the sequence (hereinafter also termed ‘heterologous sequence’) may be codon-optimised as is known in the art, in order to increase the chances that the heterologous sequence is properly expressed after introduction in the host cell.

The below table shows examples of host cells, the NHEJ actity(ies) they lack and where suitable heterologous genes can be found for restoring the NHEJ pathway.

TABLE 2
overview of suitable heterologous genes for host cells lacking various NHEJ activities.

| | | Suitable heterologous |
|---|---|---|
| | | genes can be found in |
| Host cell | Lacking activity(ies) | (non-exhaustive list) |
| Streptomyces griseus, | DNA-binding | Mycobacterium tuberculosis |
| Streptomyces | Ligase | H37Rv, Mycobacterium |
| acidiscabies, | Primase | canettii, Mycobacterium |
| Streptomyces auratus, | Polymerase | spp., Rhodococcus |
| Streptomyces | | erythropolis, Rhodococcus |
| bottropensis, | | equi, Rhodococcus fascians, |
| Streptomyces chartreusis, | | Rhodococcus rhodochrous, |
| Streptomyces | | Rhodococcus |
| clavuligerus, | | spp., Nocardia araoensis, |
| Streptomyces | | Nocardia transvalensis, |
| coelicoflavus, | | Nocardia exalbida, Nocardia |
| Streptomyces gancidicus, | | spp., Tomitella biformata, |
| Streptomyces ghanaensis, | | Amycolatopsis mediterranei, |
| Streptomyces globisporus, | | Amycolatopsis |
| Streptomyces | | orientalis, Saccharopolyspora |
| griseoaurantiacus, | | erythraea, Pseudonocardia |
| Streptomyces | | dioxanivorans, |
| griseoflavus, | | Ralstonia pickettii, Kribbelle |
| Streptomyces | | flavida, Saccharothrix |
| himastatinicus, | | espanaensis, Sinorhizobium |
| Streptomyces ipomoeae, | | meliloti, Actinoplanes |
| Streptomyces lividans, | | friuliensis, Stenotrophomonas |
| Streptomyces | | maltophilia, |
| mobaraensis, | | Sinorhizobium meliloti, |
| Streptomyces | | Rhodococcus jostii, Blastococcus |
| pristinaespiralis, | | saxobsidens, |
| Streptomyces prunicolor, | | Beutenbergia cavernae, |
| Streptomyces rimosus | | Streptomyces collinus, |
| subsp. rimosus, | | Arthrobacter phenanthrenivorans, |
| Streptomyces | | Arthrobacter |
| roseosporus, | | chlorophenolicus, Xanthomonas |
| Streptomyces | | campestris pv. |
| scabrisporus, | | raphani, Xylanimonas cellulosilytica, |
| Streptomyces | | Thermobispora |
| somaliensis, | | bispora, Sinorhizobium |
| Streptomyces sulphureus, | | medicae, Sanguibacter |
| Streptomyces sviceus, | | keddieii, Sinorhizobium |
| Streptomyces | | meliloti, Ramlibacter tataouinensis, |
| tsukubaensis, | | Intrasporangium |
| Streptomyces | | calvum |
| turgidiscabies, | | |
| Streptomyces | | |
| viridochromogenes, | | |
| Streptomyces | | |
| viridosporus, | | |
| Streptomyces | | |
| vitaminophilus, | | |
| Streptomyces | | |
| zinciresistens, | | |
| Amycolatopsis azurea, | | |
| Amycolatopsis | | |
| decaplanina, | | |
| Amycolatopsis | | |
| methanolica, | | |
| Saccharopolyspora spinosa, | | |
| Nocardia abscessus, | | |
| Nocardia aobensis, | | |
| Nocardia araoensis, | | |
| Nocardia asiatica, | | |
| Nocardia asteroides, | | |
| Nocardia brasiliensis, | | |
| Nocardia brevicatena, | | |
| Nocardia carnea, | | |
| Nocardia cerradoensis, | | |
| Nocardia concava, | | |
| Nocardia cyriacigeorgica, | | |
| Nocardia exalbida, | | |
| Nocardia higoensis, | | |
| Nocardia jiangxiensis, | | |
| Nocardia niigatensis, | | |
| Nocardia otitidiscaviarum, | | |
| Nocardia paucivorans, | | |
| Nocardia pneumoniae, | | |
| Nocardia takedensis, | | |
| Nocardia tenerifensis, | | |
| Nocardia terpenica, | | |
| Nocardia testacea, | | |
| Nocardia thailandica, | | |
| Nocardia veterana, | | |
| Nocardia vinacea, | | |
| Rhodococcus erythropolis, | | |
| Rhodococcus imtechensis, | | |
| Rhodococcus opacus, | | |
| Rhodococcus pyridinivorans, | | |
| Rhodococcus qingshengii, | | |
| Rhodococcus rhodochrous, | | |
| Rhodococcus ruber, | | |
| Rhodococcus triatomae, | | |
| Rhodococcus wratislaviensis, | | |
| Smaragdicoccus niigatensis, | | |
| Mycobacterium leprae, | | |
| Mycobacterium tuberculosis | | |
| Mycobacterium abscessus | | |
| subsp. bolletii, | | |
| Mycobacterium abscessus, | | |
| Mycobacterium avium | | |
| subsp. avium, | | |
| Mycobacterium canettii, | | |
| Mycobacterium colombiense, | | |
| Mycobacterium fortuitum | | |
| subsp. fortuitum, | | |
| Mycobacterium hassiacum, | | |
| Mycobacterium massiliense, | | |
| Mycobacterium parascrofulaceum, | | |
| Mycobacterium phlei, | | |
| Mycobacterium rhodesiae, | | |
| Mycobacterium smegmatis, | | |
| Mycobacterium thermoresistibile, | | |
| Mycobacterium tusciae, | | |
| Mycobacterium vaccae, | | |
| Mycobacterium xenopi | | |
| Streptomyces albus, | Ligase | Streptomyces carneus, |
| Streptomyces avermitilis, | | Mycobacterium tuberculosis |
| Streptomyces | | H37Rv, Mycobacterium |
| bingchenggensis, | | abscessus, Mycobacterium |
| Streptomyces coelicolor, | | canettii, Mycobacterium |
| Streptomyces pratensis, | | mageritense, Mycobacterium |
| Streptomyces | | farcinogenes, |
| rapamycinicus, | | Mycobacterium spp., |
| Streptomyces scabiei, | | Rhodococcus erythropolis, |
| Streptomyces venezuelae, | | Rhodococcus equi, Rhodococcus |
| Streptomyces | | fascians, Rhodococcus |
| violaceusniger, | | rhodochrous, |
| Frankia symbiont of Datisca | | Rhodococcus pyridinivorans, |
| glomerata, | | Rhodococcus rhodnil, |
| Rhodococcus equi, | | Rhodococcus spp., |
| | | Nocardia araoensis, Nocardia |
| | | transvalensis, Nocardia |
| | | exalbida, Nocardia |
| | | spp., Gordonia polyisoprenivorans, |
| | | Gordonia |
| | | spp., Smaragdicoccus |
| | | niigatensis, |
| Frankia symbiont of Datisca | Primase and Polymerase | Streptomyces carneus, |
| glomerata, | | Mycobacterium tuberculosis |
| Rhodococcus equi, | | H37Rv, Mycobacterium |
| | | canettii, Mycobacterium |
| | | orygis, Mycobacterium |
| | | spp., Rhodococcus |
| | | erythropolis, Rhodococcus |
| | | equi, Rhodococcus ruber, |
| | | Rhodococcus pyridinivorans, |
| | | Rhodococcus fascians, |
| | | Rhodococcus rhodochrous, |
| | | Rhodococcus fascians |
| | | Rhodococcus spp., |
| | | Nocardia thailandica, Nocardia |
| | | exalbida, Nocardia |
| | | asteroides, Nocardia vinacea, |
| | | Nocardia spp. Amycolicicoccus |
| | | subflavus, |
| | | Tomitella biformata, Smaragdicoccus |
| | | niigatensis |
| Streptomyces scabiei | DNA-binding | Mycobacterium tuberculosis |
| | | H37Rv, Mycobacterium |
| | | africanum, Mycobacterium |
| | | canettii, Mycobacterium |
| | | spp. Streptomyces |
| | | coelicolor, Streptomyces |
| | | cattleya, Streptomyces |
| | | purpureus, Streptomyces |
| | | varsoviensis, Streptomyces |
| | | thermolilacinus, Streptomyces |
| | | roseoverticillatus, |
| | | Streptomyces venezuelae, |
| | | Streptomyces spp. Amycolatopsis |
| | | mediterranei, |
| | | Amycolatopsis halophila, |
| | | Amycolatopsis vancoresmycina, |
| | | Amycolatopsis |
| | | orientalis, Amycolicicoccus |
| | | subflavus, Amycolatopsis |
| | | spp., Nakamurella |
| | | multipartita, Beutenbergia |
| | | cavernae, Arthrobacter |
| | | castelli, Saxeibacter |
| | | lacteus, Rhodococcus |
| | | equi, Nocardia jiangxiensis, |
| | | Gordonia rubripertincta, |
| | | Clavibacter |
| | | michiganensis, Gordonia |
| | | aichiensis, Microbacterium |
| | | paraoxydans |

In one embodiment, the host cell is S. coelicolor. This organism lacks the ligase activity of the NHEJ pathway and only displays the DNA-binding activity via the Ku proteins and the primase and polymerase activity (SEQ ID NO: 70). In one embodiment, NHEJ is restored in S. coelicolor by introducing at least part of the ligD gene from S. carneus, wherein said part encodes the ligase activity. In other embodiments, NHEJ is restored by introducing the ligD gene from M. tuberculosis, Nocardia spp., Smaragdicoccus niigatensis, Rhodococcus spp., Mycobacterium abscessus, Mycobacterium mageritense or Mycobacterium farcinogenes.

Target Nucleic Acid

The method disclosed herein is particularly useful for generating precise indels around at least one target nucleic acid sequence of interest. The method is thus useful for, but not limited to, the investigation of pathway regulations and the identification of metabolite production bottlenecks, the screening of producer strains and the identification of new compounds produced by the host cell.

The target nucleic acid sequence may be comprised within any nucleic acid sequence of interest. For example, the target sequence may be comprised within or may comprise an open reading frame or a putative open reading frame, or it may be comprised within or may comprise a regulatory region or a putative regulatory region, such as an enhancer, a promoter, an insulator, a terminator.

The target nucleic acid sequence may be involved in a pathway of interest. In some embodiments, the target nucleic acid encodes an enzyme or a protein. In other embodiments, the target nucleic acid is comprised within or comprises a biosynthetic gene or a putative biosynthetic gene. In some embodiments, the biosynthetic gene is involved in the synthesis of a secondary metabolite.

In some embodiments, the target nucleic acid sequence is comprised within a gene cluster. In specific embodiments, the gene cluster is a secondary metabolite gene cluster.

There is thus disclosed herein a method for generating precise indels such at precise deletions or precise insertions around a target nucleic acid sequence optionally comprised within or comprising a gene cluster, where the target nucleic acid sequence is involved or is suspected of being involved in the biosynthesis of a secondary metabolite.

In some embodiments, the secondary metabolite is selected from the group consisting of antibiotics, herbicides, anti-cancer agents, immunosuppressants, flavors, parasiticides and proteins. The term ‘parasiticide’ is to be understood in its broadest sense as an agent capable of inactivating or killing any undesirable organism and thus comprises insecticides, anthelmintic compounds, larvacides, antiparasitic agents and antiprotozoal agents.

In some embodiments, the secondary metabolite is an antibiotic selected from the group consisting of apramycin, bacitracin, chloramphenicol cephalosporins, cycloserine, erythromycin, fosfomycin, gentamicin, kanamycin, kirromycin, lassomycin, lincomycin, lysolipin, microbisporicin, neomycin, noviobiocin, nystatin, nitrofurantoin, platensimycin, pristinamycins, rifamycin, streptomycin, teicoplanin, tetracycline, tinidazole, ribostamycin, daptomycin, vancomycin, viomycin and virginiamycin.

In other embodiments, the secondary metabolite is a herbicide selected from the group consisting of bialaphos, resormycin and phosphinothricin.

In yet other embodiments, the secondary metabolite is an anti-cancer agent selected from the group consisting of doxorubicin, salinosporamides, aclarubicin, pentostatin, peplomycin, thrazarine and neocarcinostatin.

In yet other embodiments, the secondary metabolite is an immunosuppressant selected from the group consisting of rapamycin, FK520, FK506, cyclosporine, ushikulides, pentalenolactone I and hygromycin A.

In yet other embodiments, the secondary metabolite is a flavor such as geosmin.

In yet other embodiments, the secondary metabolite is a parasiticide such as an insecticide, an anthelmintic, a larvacide, or an antiprotozoal agent such as spinsad or avermectin.

In other embodiments, the target nucleic acid encodes an enzyme such as a metabolic enzyme selected from the group consisting of an amylase, a protease, a cellulase, a chitinase, a keratinase and a xylanase, a glycosyltransferase, an oxygenase, a hydroxylase, a methyltransferase, a dehydrogenase, a dehydratase.

In some embodiments, only one target nucleic acid sequence is targeted for editing and generation of precise indels. In other embodiments, more than one target nucleic acid sequence is targeted and the method is a multiplex method. Thus the method can be used for generating at least one indel around at least one target nucleic acid sequence, such as at least two indels around at least two target nucleic acid sequences, such as at least three indels around at least three target nucleic acid sequences, such as at least four indels around at least four target nucleic acid sequences, such as at least five indels around at least five target nucleic acid sequences, or more. The method can thus be used for generating one indel around one target nucleic acid sequence, or two indels around at least two target nucleic acid sequences, or three indels around three target nucleic acid sequences, or four indels around four target nucleic acid sequences, or five indels around five target nucleic acid sequences, or more. As explained above, in the case of multiplex editing, a guiding means is preferably provided for each target nucleic acid sequence.

In some embodiments, the at least one indel results in the inactivation of at least one gene. In some embodiments, the at least one gene is comprised within a gene cluster. In other embodiments, the at least one gene is not comprised within a gene cluster.

The at least one indel generated by the present method is an indel of at least 1 bp.

Efficiency and Off-Target Effects

Several parameters can have an impact on the efficiency of the present method for generating precise indels around at least one target sequence. Some parameters can be adjusted as known in the art. Parameters susceptible of having an impact on the efficiency include, but are not limited to: the sequence of the guiding means (sgRNA or crRNA/tracrRNA), the sequence of the target nucleic acid, the GC content of the host cell and the GC content of the target nucleic acid sequence.

The method for generating precise indels around a target nucleic acid sequence described herein can be performed with high efficiency, with relatively few off-target effects. In some embodiments, the desired indel is generated in more than 65% of the host cells, such as in more than 70% of the host cells, such as in more than 75% of the host cells, such as in more than 80% of the host cells, such as in more than 85% of the host cells, such as in more than 90% of the host cells, such as in more than 95% of the host cells, such as in 100% of the host cells.

Without being bound by theory, the use of homology arms to direct the repair of the break generated by the Cas9 nuclease towards the HR pathway is believed to reduce the occurrence of off-target effects. When homology arms are used, higher efficiency can be achieved, so that the desired indel is generated in more than 90% of the host cells, such as in more than 95% of the host cells, such as in more than 96% of the host cells, such as in more than 97% of the host cells, such as in more than 98% of the host cells, such as in more than 99% of the host cells, such as in 100% of the host cells.

Characterisation and Screening

The present method can thus be used for generating precise indels around a target nucleic acid sequence of interest, for example a sequence encoding for a gene involved in a pathway of interest. This can result in a plurality of clones having precise indels around the target sequence. These clones can then be further analysed or screened. For example, producer strains having advantageous production profiles for a desired compound can be selected.

In some embodiments, it may be of interest to determine the size of the at least one indel for a particular clone. Thus the method may comprise a further step of determining the size of the at least one indel. Methods for determining the size of an indel are known in the art and include, but are not limited to, whole genome sequencing, pulsed field gel electrophoresis, nucleic acid amplification-based methods such as PCR, for example followed by restriction analysis and detection of the PCR products on a gel and determination of the size of the products using an appropriate marker. The PCR products can also be sequenced if precise determination of the size of the indel is desired.

In some embodiments, the method further comprises the selection of clones having the desired characteristics. Such selection methods are known in the art and encompass screening methods, chemical analysis of the related gene products (proteins or metabolites), sequencing of the related gene regions, and/or analysis of the gene expression level.

CRISPR-Cas9 System for Actinomycetes

The most studied CRISPR-Cas9 system is from Streptococcus pyogenes, which has a GC content of about 35%. In contrast, actinomycetes have a high GC content. S. coelicolor for example has a GC content of about 72%. Likewise, codon usage varies from organism to organism.

Herein is thus disclosed a codon optimised nucleic acid sequence encoding Cas9 which is codon optimised for streptomycetes (SEQ ID NO: 1). The optimisation was done based on the codon usage table of the most studied actinomycete, Streptomyces coelicolor, as described in example 1.

In one aspect, the invention thus relates to a polynucleotide having at least 94% identity with SEQ ID NO: 1, such as at least 95% identity, such as at least 96% identity, such as at least 97% identity, such as at least 98% identity, such as at least 99% identity, such as 100% identity, said polynucleotide encoding a Cas9 nuclease or a variant thereof. It will be understood that sequences closely related to SEQ ID NO: 1 with mutations such as e.g. silent mutations are envisaged.

In some embodiments, the polynucleotide is non-naturally occurring.

Also within the scope of the present disclosure is a polypeptide encoded by a polynucleotide having at least 94% identity with SEQ ID NO: 1, such as at least 95% identity, such as at least 96% identity, such as at least 97% identity, such as at least 98% identity, such as at least 99% identity, such as 100% identity with SEQ ID NO: 1. In one embodiment, the polypeptide has the sequence as set forth in SEQ ID NO: 2.

It will be understood that sequences closely related to SEQ ID NO: 2 with mutations that do not disrupt the function of Cas9 are also within the scope of the invention. In particular, mutations in non-conserved domains of Cas9 which are unlikely to affect its function and conservative mutations in conserved or non-conserved domains of Cas9 are envisaged.

In some embodiments, the polypeptide is non-naturally occurring.

Also within the scope of the present disclosure is a cell comprising the polynucleotide disclosed herein. Such a cell may be a host cell as detailed above. In particular, the cell may be an archaea, in a prokaryotic cell or in a eukaryotic cell. In one embodiment, the host cell is a prokaryotic cell. The host cell may be a cell with a high GC content, for example a GC content of 50% or more, such as 55% or more, such as 60% or more, such as 65% or more, such as 70% or more, such as 75% or more, such as 80% or more, such as 85% or more, such as 90% or more. In a particular embodiment, the host cell is an actinobacterium. The host cell may thus be selected from the group consisting of Actinomycetales, such as Streptomyces sp., Amycolatopsis sp. or Saccharopolyspora sp. In some embodiments, the host cell is selected from the group consisting of Streptomyces coelicolor, Streptomyces avermitilis, Streptomyces aureofaciens, Streptomyces griseus, Streptomyces parvulus, Streptomyces albus, Streptomyces vinaceus, Streptomyces acrimycinis, Streptomyces calvuligerus, Streptomyces lividans, Streptomyces limosus, Streptomyces rubiqinosis, Streptomyces azureus, Streptomyces glaucenscens, Streptomyces rimosus, Streptomyces violaceoruber, Streptomyces kanamyceticus, Amycolatopsis orientalis, Amycolatopsis mediterranei, Saccharopolyspora erythraea, Mycobacterium tuberculosis, Streptomyces carneus, Nocardia spp., Smaragdicoccus niigatensis, Rhodococcus spp., Mycobacterium abscessus, Mycobacterium mageritense, Mycobacterium farcinogenes. In a preferred embodiment, the host cell is Streptomyces coelicolor.

The present disclosure also relates to a vector comprising the polynucleotide as described herein. Thus some embodiments relate to a vector comprising a polynucleotide having at least 94% identity with SEQ ID NO: 1, such as at least 95% identity, such as at least 96% identity, such as at least 97% identity, such as at least 98% identity, such as at least 99% identity, such as 100% identity with SEQ ID NO: 1.

The polynucleotide, the polypeptide and/or the vector comprising the polynucleotide, as all disclosed herein, may be used for performing the methods disclosed herein. In preferred embodiments, they are used to perform the present methods in a host cell, where the host cell is a Streptomycetes.

In some embodiments, the method is a method for generating at least one deletion around at least one target nucleic acid sequence comprised within a host cell having a non-homologous end-joining (NHEJ) pathway which is at least partly deficient,
said method comprising the steps of:
(i) optionally, restoring the full functionality of the NHEJ pathway,
(ii) inducing a CRISPR-Cas9 system in said host cell, wherein said CRISPR-Cas9 system is able to generate at least one break in said at least one target nucleic acid sequence and wherein the CRISPR-Cas9 system comprises a Cas9 nuclease and at least one guiding means,
thereby generating:
a. if the method does not comprise step (i), at least one random-sized deletion around said at least one target nucleic acid sequence, wherein said at least one deletion is a random-sized deletion of at least 1 bp; or
b. if the method does comprise step (i), at least one indel around said at least one target nucleic acid sequence, wherein said at least one indel is a deletion or insertion of at least1 bp,

wherein Cas9 is a polypeptide as described above, or wherein Cas9 is encoded by a polynucleotide as described above.

Accordingly, in some embodiments, the method does not comprise step (i) of restoring the full functionality of the NHEJ pathway and results in generation of random-sized deletions, where Cas9 is a polypeptide encoded by a polynucleotide having at least 94% identity with SEQ ID NO: 1, such as at least 95% identity, such as at least 96% identity, such as at least 97% identity, such as at least 98% identity, such as at least 99% identity, such as 100% identity with SEQ ID NO: 1. In one embodiment, the polypeptide has the sequence as set forth in SEQ ID NO: 2. In some embodiments, the polynucleotide encoding Cas9 is codon-optimised for the host cell in which the method is to be performed.

In other embodiments, the method comprises step (i) of restoring the full functionality of the NHEJ pathway and results in generation of indels, i.e. insertions of deletions of at least 1 bp, where Cas9 is a polypeptide encoded by a polynucleotide having at least 94% identity with SEQ ID NO: 1, such as at least 95% identity, such as at least 96% identity, such as at least 97% identity, such as at least 98% identity, such as at least 99% identity, such as 100% identity with SEQ ID NO: 1. In one embodiment, the polypeptide has the sequence as set forth in SEQ ID NO: 2. In some embodiments, the polynucleotide encoding Cas9 is codon-optimised for the host cell in which the method is to be performed.

Method for Selective Modulation of Transcription

In another aspect, a method for selectively modulating transcription of at least one target nucleic acid sequence in a host cell is disclosed, the method comprising introducing into the host cell:
i. at least one guiding means, or a nucleic acid comprising a nucleotide sequence encoding guiding means, wherein the guiding means comprises a nucleotide sequence that is complementary to a target nucleic acid sequence in the host cell; and
ii. a variant Cas9, or a nucleic acid comprising a nucleotide sequence encoding the variant Cas9, wherein the variant Cas9 has reduced endodeoxyribonuclease activity,

wherein said guiding means and said variant Cas9 form a complex in the host cell, said complex selectively modulating transcription of at least one target nucleic acid in the host cell.

In some embodiments, the method for selectively modulating transcription of at least one target nucleic acid sequence in a host cell comprises introducing into the host cell:
(i) at least one guiding means, or a nucleic acid comprising a nucleotide sequence encoding guiding means, wherein the guiding means comprises a nucleotide sequence that is complementary to a target nucleic acid sequence in the host cell; and
(ii) a variant Cas9, or a nucleic acid comprising a nucleotide sequence encoding the variant Cas9, wherein the variant Cas9 is a variant of the polypeptides disclosed herein or of a polypeptide encoded by the nucleotide sequences disclosed herein, and wherein the variant Cas9 has reduced endodeoxyribonuclease activity, with reduced endodeoxyribonuclease activity and is codon-optimised for Streptomycetes,
wherein said guiding means and said variant Cas9 form a complex in the host cell, said complex selectively modulating transcription of at least one target nucleic acid in the host cell.

In some embodiments, the guiding means comprises at least one sgRNA and/or at least one crRNA/tracrRNA set.

Modulation

This method allows selective modulation of the transcription of at least one target nucleic acid sequence comprised within a host cell.

Modulation of the transcription can be an increase of the transcription level or a decrease of the transcription level.

The method for modulation of transcription is based on the use of a CRISPR-Cas9 system comprising a variant Cas9 and at least one guiding means, wherein the variant Cas9 is capable of forming a complex with each of the at least one guiding means and is thereby capable of binding to the target nucleic acid sequence but is not capable of inducing a break therein or is not capable of leaving the target nucleic acid sequence. In other words, variant Cas9 remains on the target nucleic acid sequence, whereby it is hypothesized that transcription is prevented because of steric hindrance or lower accessibility of a polymerase such as an RNA polymerase to the DNA. In order to achieve an increase of transcription, a transcription activator can be fused to the variant Cas9, wherein the variant Cas9 is capable of forming a complex with at least one guiding means targeting e.g. the promoter of a gene of interest; the complex remains on the target nucleic acid sequence and thereby provides a transcription activator, thereby activating expression of the gene.

In some embodiments, the variant Cas9 is a variant Cas9 which can cleave one of the strands of the target nucleic acid sequence but has reduced ability to cleave the other strand of the target nucleic acid sequence. In some embodiments, the variant Cas9 is selected from the group consisting of Cas9-H840A, Cas9-D10A and Cas9-H840A, D10A, where H840A indicates a substitution at amino acid residue 840 of SEQ ID NO: 2, and D10A indicates a substitution at amino acid residue 10 of Cas9. It will be understood that sequences having mutations that do not disrupt the function of the variant Cas9 are also within the scope of the invention. In particular, mutations in non-conserved domains of Cas9 which are unlikely to affect its function and conservative mutations in conserved or non-conserved domains of Cas9 are envisaged.

In some embodiments, the expression of the variant Cas9 is inducible, e.g. the nucleic acid sequence encoding the variant Cas9 may be under the control of an inducible promoter. Other methods of inducing expression of the variant Cas9 will be apparent to the skilled person.

In some embodiments, the nucleic acid sequence encoding the variant Cas9 is comprised within a vector to be introduced in the host cell. In other embodiments, the nucleic acid sequence encoding the variant Cas9 is comprised within the genome of the host cell, e.g. on a chromosome.

The CRISPR-Cas9 system preferably further comprises at least one guiding means allowing the variant Cas9 to bind to the at least one target nucleic acid sequence and to modulate its transcription. As detailed above, the nucleic acid sequence encoding the variant Cas9 and the at least one nucleic acid sequence encoding the at least one guiding means may be comprised within a single nucleic acid such as a vector or a chromosome comprised within the host cell.

Host Cell

The present method can be performed in an archaea, in a prokaryotic cell or in a eukaryotic cell. In one embodiment, the host cell is a prokaryotic cell. The present methods are particularly advantageous for modulating transcription in host cells that have a high GC content, for example a GC content of 50% or more, such as 55% or more, such as 60% or more, such as 65% or more, such as 70% or more, such as 75% or more, such as 80% or more. In a particular embodiment, the host cell is an actinobacterium. The host cell may thus be selected from the group consisting of Actinomycetales, such as Streptomyces sp., Amycolatopsis sp. or Saccharopolyspora sp. In some embodiments, the host cell is selected from the group consisting of Streptomyces coelicolor, Streptomyces avermitilis, Streptomyces aureofaciens, Streptomyces griseus, Streptomyces parvulus, Streptomyces albus, Streptomyces vinaceus, Streptomyces acrimycinis, Streptomyces calvuligerus, Streptomyces lividans, Streptomyces limosus, Streptomyces rubiqinosis, Streptomyces azureus, Streptomyces glaucenscens, Streptomyces rimosus, Streptomyces violaceoruber, Streptomyces kanamyceticus, Amycolatopsis orientalis, Amycolatopsis mediterranei, Saccharopolyspora erythraea, Mycobacterium tuberculosis, Streptomyces carneus, Nocardia spp., Smaragdicoccus niigatensis, Rhodococcus spp., Mycobacterium abscessus, Mycobacterium mageritense, Mycobacterium farcinogenes. In a preferred embodiment, the host cell is Streptomyces coelicolor.

The host cell may be any of the organisms listed herein elsewhere.

Target Nucleic Acid

The method disclosed herein is particularly useful for modulating transcription of least one target nucleic acid sequence of interest. The method is thus useful for, but not limited to, the investigation of pathway regulations and identification of metabolite production bottlenecks, the design of producer strains and the identification of new compounds produced by the host cell.

The target nucleic acid sequence may be comprised within any nucleic acid sequence of interest. For example, the target sequence may be comprised within or may comprise an open reading frame or a putative open reading frame, or it may be comprised within or may comprise a regulatory region or a putative regulatory region, such as an enhancer, a promoter, an insulator, a terminator.

The target nucleic acid sequence may be involved in a pathway of interest. In some embodiments, the target nucleic acid encodes an enzyme. In other embodiments, the target nucleic acid is comprised within or comprises a biosynthetic gene or a putative biosynthetic gene. In some embodiments, the biosynthetic gene is involved in the synthesis of a secondary metabolite.

In some embodiments, the target nucleic acid sequence is comprised within a gene cluster. In specific embodiments, the gene cluster is a secondary metabolite gene cluster.

There is thus disclosed herein a method for modulating transcription of at least one target nucleic acid sequence optionally comprised within or comprising a gene cluster, where the target nucleic acid sequence is involved or is suspected of being involved in the biosynthesis of a secondary metabolite.

In some embodiments, the secondary metabolite is selected from the group consisting of antibiotics, herbicides, anti-cancer agents, immunosuppressants, flavors, parasiticides, enzymes and proteins. The term ‘parasiticide’ is to be understood in its broadest sense as an agent capable of inactivating or killing any undesirable organism and thus comprises insecticides, anthelmintic compounds, larvacides, antiparasitic agents and antiprotozoal agents.

In some embodiments, the secondary metabolite is an antibiotic selected from the group consisting of apramycin, bacitracin, chloramphenicol cephalosporins, cycloserine, erythromycin, fosfomycin, gentamicin, kanamycin, kirromycin, lassomycin, lincomycin, lysolipin, microbisporicin, neomycin, noviobiocin, nystatin, nitrofurantoin, platensimycin, pristinamycins, rifamycin, streptomycin, teicoplanin, tetracycline, tinidazole, ribostamycin, daptomycin, vancomycin, viomycin and virginiamycin.

In other embodiments, the secondary metabolite is a herbicide selected from the group consisting of bialaphos, resormycin and phosphinothricin.

In yet other embodiments, the secondary metabolite is an anti-cancer agent selected from the group consisting of doxorubicin, salinosporamides, aclarubicin, pentostatin, peplomycin, thrazarine and neocarcinostatin.

In yet other embodiments, the secondary metabolite is an immunosuppressant selected from the group consisting of rapamycin, FK520, FK506, cyclosporine, ushikulides, pentalenolactone I and hygromycin A.

In yet other embodiments, the secondary metabolite is a flavor such as geosmin.

In yet other embodiments, the secondary metabolite is a parasiticide such as an insecticide, an anthelmintic, a larvacide, or an antiprotozoal agent such as spinsad or avermectin.

In other embodiments, the target nucleic acid encodes an enzyme such as metabolic enzyme selected from the group consisting of an amylase, a protease, a cellulase, a chitinase, a keratinase and a xylanase, a glycosyltransferase, an oxygenase, a hydroxylase, a methyltransferase, a dehydrogenase, a dehydratase.

In some embodiments, transcription of only one target nucleic acid sequence is modulated. In other embodiments, transcription of more than one target nucleic acid sequence is modulated and the method is a multiplex method. Thus the method can be used for modulating transcription of at least one target nucleic acid sequence, such as of least two target nucleic acid sequences, such as of at least three target nucleic acid sequences, such as of at least four target nucleic acid sequences, such as of at least five target nucleic acid sequences, or more. The method can thus be used for modulating transcription of one target nucleic acid sequence, of two target nucleic acid sequences, of three target nucleic acid sequences, of four target nucleic acid sequences, of five target nucleic acid sequences, or more. As explained above, in the case of multiplex modulation, a guiding means is preferably provided for each target nucleic acid sequence.

In some embodiments, the at least one nucleic acid sequence is at least one gene. The gene may be comprised within a gene cluster. In other embodiments, the at least one gene is not comprised within a gene cluster.

Kits

Kit for Generating Random-Sized Deletions and/or Indels

In a further aspect, the disclosure relates to a kit for performing the methods described herein.

In some embodiments, the kit is for generating at least one random-sized deletion around at least one target nucleic acid sequence described above, said kit comprising a vector comprising a nucleic acid sequence encoding a Cas9 nuclease or a variant thereof and instructions for use.

The vector comprised within said kit can be an integrative vector for integrating the nucleic acid sequence encoding the nuclease into the genome, or it can be comprised within a non-integrative vector, e.g. to be used as a template for amplifying the nucleic acid sequence encoding the nuclease prior to introduction into the cell, or to be transformed and maintained in the host cell.

In preferred embodiments, the nuclease is Cas9 or a variant thereof. In some embodiments, the nucleic acid sequence encoding the nuclease is a sequence encoding Cas9 such as a polynucleotide having at least 93% identity with SEQ ID NO: 1, such as at least 94% identity, such as at least 95% identity, such as at least 96% identity, such as at least 97% identity, such as at least 98% identity, such as at least 99% identity, such as 100% identity with SEQ ID NO: 1.

The kit may further comprise at least one guiding means and/or at least one host cell having a non-homologous end-joining (NHEJ) pathway which is at least partly deficient.

In some embodiments, the kit further comprises at least one guiding means, where the guiding means is as described above. The guiding means may be comprised within the vector or it may be provided on a different vector. The at least one guiding means may be any guiding means described above, such as an sgRNA or a crRNA/tracrRNA set.

In some embodiments, the kit further comprises a host cell or a plurality of host cells. In one embodiment, the host cell is a cell having a partly deficient NHEJ pathway, i.e. lacking at least one of the four NHEJ activities defined above. The host cell may be any of the host cells described herein elsewhere. The NHEJ pathway may be partly deficient because it is naturally partly deficient in said host cell, or it may have been inactivated by the manufacturer or by the user. In one embodiment, the host cell is S. coelicolor and lacks the ligase activity.

In other embodiments, the host cell has a functional NHEJ pathway. The kit may then further comprise means for at least partly inactivating the NHEJ pathway in said host cell. This can be done as described above, i.e. by inactivating at least one of the four NHEJ activities (DNA binding, ligase, polymerase or primase activity). Thus in one embodiment the kit comprises means for inactivating the ligase activity of the host cell.

In some embodiments, the kit is for performing the method for generating at least one precise indel around at least one target nucleic acid sequence, said kit comprising a first vector comprising a nucleic acid sequence encoding Cas9 or a variant thereof and instructions for use.

In some embodiments, the nucleic acid sequence encoding Cas9 is a polynucleotide having at least 93% identity with SEQ ID NO: 1, such as at least 94% identity, such as at least 95% identity, such as at least 96% identity, such as at least 97% identity, such as at least 98% identity, such as at least 99% identity, such as 100% identity with SEQ ID NO: 1.

In some embodiments, the kit further comprises at least one guiding means, where the guiding means is as described above. The guiding means may be comprised within the first vector or it may be provided on a different vector. The at least one guiding means may be any guiding means described above, such as an sgRNA or a crRNA/tracrRNA set.

In some embodiments, the kit further comprises a host cell or a plurality of host cells. In one embodiment, the host cell is a cell having a partly deficient NHEJ pathway, i.e. lacking at least one of the four NHEJ activities defined above. The host cell may be any of the host cells described herein elsewhere. The NHEJ pathway may be partly deficient because it is naturally partly deficient in said host cell, or it may have been inactivated by the manufacturer. In one embodiment, the host cell is S. coelicolor and lacks the ligase activity.

In other embodiments, the host cell has a functional NHEJ pathway. The kit may then further comprise means for at least partly inactivating the NHEJ pathway in said host cell. This can be done as described above, i.e. by inactivating at least one of the four NHEJ activities (DNA binding, ligase, polymerase or primase activity). Thus in one embodiment the kit comprises means for inactivating the ligase activity of the host cell.

In some embodiments, the kit further comprises a second vector comprising a nucleic acid sequence encoding at least one of the four NHEJ activities defined above. In one embodiment, the nucleic acid thus encodes at least one of:
a DNA-binding activity,
a primase activity,
a ligase activity,
a polymerase activitiy.

In some embodiments, the nucleic acid sequence encodes two or three of the four NHEJ activities. In some embodiments, the nucleic acid sequence encodes all four NHEJ activities. In some embodiments, the nucleic acid sequence encodes the ligase D from S. carneus or M. tuberculosis. In a particular embodiment, the host cell is S. coelicolor and the nucleic acid sequence encoding the missing NH EJ activity comprises the ligase D gene from S. carneus or M. tuberculosis. Examples of which organisms having sequences that can be used for restoring NH EJ activity are provided above (Table 2).

In other embodiments, the nucleic acid sequence encoding at least one of the four NEHJ activities and the nucleic acid sequence encoding Cas9 are all comprised within the first vector.

Kit for Modulating Transcription

In yet another aspect is disclosed a kit for performing the method for modulating transcription of at least one target nucleic acid as described above, said kit comprising a vector comprising a nucleic acid sequence encoding a variant Cas9; and instructions for use. In preferred embodiments, the variant Cas9 has reduced endodeoxyribonuclease activity.

In some embodiments, the variant Cas9 is a variant Cas9 which can cleave one of the strands of the target nucleic acid sequence but has reduced ability to cleave the other strand of the target nucleic acid sequence. In some embodiments, the variant Cas9 is selected from the group consisting of Cas9-H840A, Cas9-D10A and Cas9-H840A, D10A, where H840A indicates a substitution at amino acid residue 840 of SEQ ID NO: 2, and D10A indicates a substitution at amino acid residue 10 of Cas9. It will be understood that sequences having mutations that do not disrupt the function of the variant Cas9 are also within the scope of the invention. In particular, mutations in non-conserved domains of Cas9 which are unlikely to affect its function and conservative mutations in conserved or non-conserved domains of Cas9 are envisaged.

In some embodiments, the kit further comprises at least one guiding means, where the guiding means is as described above, and/or at least one host cell or plurality of host cells. The guiding means may be comprised within the first vector or it may be provided on a different vector. The at least one guiding means may be any guiding means described above, such as an sgRNA or a crRNA/tracrRNA set.

The host cell may be an archaea, in a prokaryotic cell or in a eukaryotic cell. In one embodiment, the host cell is a prokaryotic cell. The present methods can be used for modulating transcription in host cells that have a high GC content, for example a GC content of 50% or more, such as 55% or more, such as 60% or more, such as 65% or more, such as 70% or more, such as 75% or more, such as 80% or more. In a particular embodiment, the host cell is an actinobacterium. The host cell may thus be selected from the group consisting of Actinomycetales, such as Streptomyces sp., Amycolatopsis sp. or Saccharopolyspora sp. In some embodiments, the host cell is selected from the group consisting of Streptomyces coelicolor, Streptomyces avermitilis, Streptomyces aureofaciens, Streptomyces griseus, Streptomyces parvulus, Streptomyces albus, Streptomyces vinaceus, Streptomyces acrimycinis, Streptomyces calvuligerus, Streptomyces lividans, Streptomyces limosus, Streptomyces rubiqinosis, Streptomyces azureus, Streptomyces glaucenscens, Streptomyces rimosus, Streptomyces violaceoruber, Streptomyces kanamyceticus, Amycolatopsis orientalis, Amycolatopsis mediterranei, Saccharopolyspora erythraea, Mycobacterium tuberculosis, Streptomyces carneus, Nocardia spp., Smaragdicoccus niigatensis, Rhodococcus spp., Mycobacterium abscessus, Mycobacterium mageritense, Mycobacterium farcinogenes. In a preferred embodiment, the host cell is Streptomyces coelicolor.

### EXAMPLES

### Example 1

### Materials and Methods

Strains and Chemicals

ISP2: Yeast Extract, 0.4%, Malt Extract, 1%, Dextrose, 0.4%, 2% agar for solidification, pH 7.2. Cullum agar, also termed SFM (soya flour mannitol) agar: 2% organic soya flour (low fat), 2% mannitol, 2% agar, 10 mM MgCl₂, natural pH. LB: Tryptone, 1%, Yeast Extract, 0.5%, NaCl, 0.5%, pH, 7.0. 2xYT: Tryptone, 1.6%, Yeast Extract, 1%, NaCl, 0.5%, pH 7.

Chemicals and solutions: apramycin sulfate (stock solution 100 mg/ml in ddH₂O), nalidixic acid (stock solution 50 mg/ml in ddH₂O of pH 11), thiostrepton (stock solution 50 mg/ml in DMSO), kanamycin (stock solution 50 mg/ml in ddH₂O), chloramphenicol (stock solution 50 mg/ml in ethanol), chloroform, methanol, and DMSO. The working concentrations for apramycin, nalidixic acid, thiostrepton, kanamycin, and chloramphenicol were 50 μg/ml, 50 μg/ml, 1 μg/ml, 25 μg/ml, and 25 μg/ml, respectively.

The below tables list selected target sequences (Table 3), primers (Table 4) and strains and plasmids (table 5) used in the following examples.

TABLE 3
Selected target sequences

| sgRNA | The target Sequences | PAM | Purpose |
|---|---|---|---|
| Actlorf1-1 NT | GTGGCTCGAAGGAGGCTCGA | AGG | Gene deletion/ex- |
| | | | pression control |
| Actlorf1-2 T | AGCTCGATCAAGTCGATGGT | CGG | Gene deletion/ex-pression control |
| Actlorf1-3 T | GAAGCGCAGAGTCGTCATCA | CGG | Gene deletion/ex-pression control |
| Actlorf1-4 T | CCCCTCGCCCTACCGTTCAC | AGG | Gene deletion/ex- |
| | | | pression control |
| Actlorf1-5 T | GCGCGAGTATCTGCTGCTGT | CGG | Gene deletion |
| Actlorf1-6 T | CTGCAACGCGTACCACATGA | CGG | Gene deletion |
| Actvb-1 NT | TCGCCGCAACTGTCGAACAC | CGG | Gene deletion |
| Actvb-2 NT | CTGCCATCTTCGAACTCCCT | AGG | Gene deletion |
| Actvb-3 T | TTCCCGGTGTTCGACAGTTG | CGG | Gene deletion |
| Actvb-4 T | ACTGGTCTGCCTGGCTCGTA | CGG | Gene deletion |
| Actvb-5 NT | ATCTTCGAACTCCCTAGGCG | AGG | Gene deletion |
| Actvb-6 NT | GTCCCGGAGCATTCCCTGGT | CGG | Gene deletion |
| orf1p-S1 T | GTGTTCCCCTCCCTGCCTCG | TGG | Gene expression con- |
| | | | trol |
| orf1p-S3 T | TCCCTCACGCGCTCAGCTTT | GGG | Gene expression con- |
| | | | trol |
| orf1p-S5 T | CTTTGGGCGCCCGGCTCGAG | CGG | Gene expression con- |
| | | | trol |
| orf1p-A1 NT | CCTTCGACCGCCGCTCGAGC | CGG | Gene expression con- |
| | | | trol |
| orf1p-A4 NT | GCCCAAAGCTGAGCGCGTGA | AGG | Gene expression con- |
| | | | trol |
| orf1p-A5 NT | TGAGCGCGTGAGGGACCACG | AGG | Gene expression con- |
| | | | trol |
| Actlorf1-7 NT | TGAGCAGTTCCCAGAACTGC | CGG | Gene expression con- |
| | | | trol |
| Actlorf1-8 NT | AGGAGGCTCGAAGGCCGATA | CGG | Gene expression con- |
| | | | trol |

TABLE 4
Primer list.

| Sets | Primer name | Sequence (5′-3′) #§ | Purpose |
|---|---|---|---|
| 1 | Actlorf1-F1 | CATGCCATGGGTGGCT | sgRNAs Amplification |
| | | CGAAGGAGGCTCGA | |
| | | GTTTTAGAGCTAGAAATAGC | |
| 2 | Actlorf1-F2 | CATGCCATGGAGCTCG | |
| | | ATCAAGTCGATGGTGT | |
| | | TTTAGAGCTAGAAATAGC | |
| 3 | Actlorf1-F3 | CATGCCATGGGAAGCG | |
| | | CAGAGTCGTCATCAGTT | |
| | | TTAGAGCTAGAAATAGC | |
| 4 | Actlorf1-F4 | CATGCCATGGCCCCTCG | |
| | | CCCTACCGTTCACGTTTT | |
| | | AGAGCTAGAAATAGC | |
| 5 | Actlorf1-F5 | CATGCCATGGGCGCGA | |
| | | GTATCTGCTGCTGTGTT | |
| | | TTAGAGCTAGAAATAGC | |
| 6 | Actlorf1-F6 | CATGCCATGGCTGCAAC | |
| | | GCGTACCACATGAGTT | |
| | | TTAGAGCTAGAAATAGC | |
| 7 | Actlorf1-F7 | CATGCCATGGTGAGCA | |
| | | GTTCCCAGAACTGCGTT | |
| 8 | Actlorf1-F8 | CATGCCATGGAGGAGGCT | |
| | | CGAAGGCCGATAGTT | |
| 9 | ActVB-F1 | CATGCCATGGTCGCCG | |
| | | CAACTGTCGAACACGTT | |
| | | TTAGAGCTAGAAATAGC | |
| 10 | ActVB-F2 | CATGCCATGGCTGCCAT | |
| | | CTTCGAACTCCCTGTT | |
| | | TTAGAGCTAGAAATAGC | |
| 11 | ActVB-F3 | CATGCCATGGTTCCCG | |
| | | GTGTTCGACAGTTGGTT | |
| | | TTAGAGCTAGAAATAGC | |
| 12 | ActVB-F4 | CATGCCATGGACTGGT | |
| | | CTGCCTGGCTCGTAGTT | |
| | | TTAGAGCTAGAAATAGC | |
| 13 | ActVB-F5 | CATGCCATGGATCTTCG | |
| | | AACTCCCTAGGCGGTT | |
| | | TTAGAGCTAGAAATAGC | |
| 14 | ActVB-F6 | CATGCCATGGGTCCCGG | |
| | | AGCATTCCCTGGTGTT | |
| | | TTAGAGCTAGAAATAGC | |
| 15 | orf1p-S1 T-F | CATGCCATGGGTGTTC | |
| | | CCCTCCCTGCCTCGGTT | |
| | | TTAGAGCTAGAAATAGC | |
| 16 | orf1p-S3 T-F | CATGCCATGGTCCCTCA | |
| | | CGCGCTCAGCTTTGTT | |
| | | TTAGAGCTAGAAATAGC | |
| 17 | orf1p-S5 T-F | CATGCCATGGCTTTGG | |
| | | GCGCCCGGCTCGAGGTT | |
| | | TTAGAGCTAGAAATAGC | |
| 18 | orf1p-Al NT-F | CATGCCATGGCCTTCG | |
| | | ACCGCCGCTCGAGCGTT | |
| | | TTAGAGCTAGAAATAGC | |
| 19 | orf1p-A4 NT-F | CATGCCATGGGCCCAAA | |
| | | GCTGAGCGCGTGAGTT | |
| | | TTAGAGCTAGAAATAGC | |
| 20 | orf1p-A5 NT-F | CATGCCATGGTGAGCG | |
| | | CGTGAGGGACCACGGTT | |
| | | TTAGAGCTAGAAATAGC | |
| 21 | sgRNA-R | ACGCCTACGTAAAAAAA | |
| | | GCACCGACTCGGTGCC | |
| 22 | gRNA check-F | ACATGTGCGGTCGATCTT | sgRNAs sequencing |
| 23 | gRNA check-R | TACGTAAAAAAAGCACCGAC | |
| 24 | orf1-5′F | TCGTCGAAGGCACTAGAAGG | For actlORF1 homol- |
| | | CATCCGCTGAACGAGACCC | ogous recombination |
| 25 | orf1-5'R | GCTCACGTCGAAGCGGGTG | template construction |
| | | ACCACGCAGGACTCCGAAGTC | |
| 26 | orf1-3'F | TCACCCGCTTCGACGTGAG | |
| 27 | orf1-3'R | GGTCGATCCCCGCATATAGG | |
| | | TTCGCCGAGCACCAGGTC | |
| 28 | VB-5'F | TCGTCGAAGGCACTAGAAGG | For actVB homolo- |
| | | CGACTCGCTCGCCCTGATG | gous recombination |
| 29 | VB-5'R | CACCAACCTGCTCGGGCTG | template construction |
| | | CGCCGTGGAAGTGGGTGTTGAC | |
| 30 | VB-3'F | GCAGCCCGAGCAGGTTGG | |
| 31 | VB-3'R | GGTCGATCCCCGCATATAGG | |
| | | TCCGTTGCGGCGTCCATC | |
| 32 | VB-check-F | CGGCTGGTGCGTCAGCAAC | Check actVB deletion |
| 33 | VB-check-R | ACGTGGCGGGTCGAACGG | |
| 34 | ORF1-check-F | CCGCCTTGAGGACCTGTTTG | Check actlORF1 dele- |
| 35 | ORF1-check-R | ACACGCTGACCGACTTGGG | tion |
| 36 | CAS9-check-F | TCCACGAGCACATCGCCAAC | Check cas9 sub- |
| 37 | CAS9-check-R | GACCTTGTAGTCGCCGTAGACG | cloning |
| 36 | ScaligD-F | TCGTCGAAGGCACTAGAAGGG | ScaligD expression |
| | | CGGTCGATCTTGACGGCTG | cassette amplification |
| 37 | ScaligD-R | GGTCGATCCCCGCATATAGGT | |
| | | GCCGCCGGGCGTTTTTTAT | |
| 38 | orf1-6 LigD test-F | CCGCCGACACCCCGATCACC | Check NHEJ for |
| 39 | orf1-6 ligD test-R | ACCGCAGCTTCCGCTCCCTG | actlORF1 editing |
| 40 | vb2 ligD test-F | CGAGGTGATCGACGCCAACC | Check NHEJ for |
| 41 | vb2 ligD test-R | TCGCCGAGCAGGATGATGTG | actVB editing |

#: The restriction sites are underlined; the 20 nt target sequences are shown in bold, the pattern of the sgRNA-F primer is:
CATGCCATGGN₂₀GTTTTAGAGCTAGAAATAG C.
*: The overlap sequence for Gibson assembly is shown in italic.
§: The restriction sites are underlined.

TABLE 5
Strains and plasmids

| Name | Description | Reference |
|---|---|---|
| WT | Streptomyces coelicolor A3(2) | 95 SNPs and 1 |
| | | deletions of (Bentley |
| | | et al., 2002) |
| No Target | WT with pCRISPR-Cas9 | This study |
| Mismatch | WT with sgRNA: Actlorf1-1 NT including its | This study |
| | PAM sequence | |
| Δactlorf1-1 | WT with pCRISPR-Cas9 carrying sgRNA: Actlorf1- | This study |
| | 1 NT, 1 bp insertions from the DSB site | |
| Δactlorf1-2 | WT with pCRISPR-Cas9 carrying sgRNA: Actlorf1- | This study |
| | 6 T, 10721 bp deletion around the DSB | |
| | site | |
| Δactvb-1 | WT with pCRISPR-Cas9 carrying sgRNA: | This study |
| | Actvb-2 NT, 14716 bp deletion around the DSB | |
| | site | |
| Δactvb-2 | WT with pCRISPR-Cas9 carrying sgRNA: | This study |
| | Actvb-5 NT, 37173 bp deletion around the DSB | |
| | site | |
| Δactlorf1- | WT with pCRISPR-Cas9-ScaligD carrying sgR- | This study |
| ligD1- | NA: Actlorf1-6 T, 8 random red clones | |
| Δactlorf1-ligD8 | | |
| Δactvb-ligD1- | WT with pCRISPR-Cas9-ScaligD carrying sgR- | This study |
| Δactvb-ligD8 | NA: Actvb-2 NT, 8 random red clones | |
| orf1 deletion1- | WT with actlORF1 recombination arm in the | This study |
| orf1 deletion10 | pCRISPR-Cas9 carrying sgRNA: Actlorf1-6 T, | |
| | actlORF1 gene was deleted, 10 random clones | |
| vb deletion1-vb | WT with actVB recombination arm in the | This study |
| deletion10 | pCRISPR-Cas9 carrying sgRNA: Actvb-2 NT, | |
| | actVB gene was deleted, 10 random clones | |
| orf1 knock- | WT with pCRISPR-dCas9 carrying sgRNA: | This study |
| down-1 | orf1p-S1 T | |
| orf1 knock- | WT with pCRISPR-dCas9 carrying sgRNA: | This study |
| down-2 | orf1p-S3 T | |
| orf1 knock- | WT with pCRISPR-dCas9 carrying sgRNA: | This study |
| down-3 | orf1p-S5 T | |
| orf1 knock- | WT with pCRISPR-dCas9 carrying sgRNA: | This study |
| down-4 | orf1p-A1 NT | |
| orf1 knock- | WT with pCRISPR-dCas9 carrying sgRNA: | This study |
| down-5 | orf1p-A4 NT | |
| orf1 knock- | WT with pCRISPR-dCas9 carrying sgRNA: | This study |
| down-6 | orf1p-A5 NT | |
| orf1 knock- | WT with pCRISPR-dCas9 carrying sgRNA: Actlorf1- | This study |
| down-7 | 2T | |
| orf1 knock- | WT with pCRISPR-dCas9 carrying sgRNA: Actlorf1- | This study |
| down-8 | 3T | |
| orf1 knock- | WT with pCRISPR-dCas9 carrying sgRNA: Actlorf1- | This study |
| down-9 | 4T | |
| orf1 knock- | WT with pCRISPR-dCas9 carrying sgRNA: Actlorf1- | This study |
| down-10 | 1NT | |
| orf1 knock- | WT with pCRISPR-dCas9 carrying sgRNA: Actlorf1- | This study |
| down-11 | 7NT | |
| orf1 knock- | WT with pCRISPR-dCas9 carrying sgRNA: Actlorf1- | This study |
| down-12 | 8NT | |
| ET12567/pUZ8002 | Escherichia coli for conjugation | (2) |
| | dam-13::Tn9 dcm-6 hsdM Cml^{R}, carrying helper | |
| | plasmid pUZ8002 | |
| Mach1 ™-T1^{R} | Escherichia coli for routine cloning | Life Technologies |
| | lacZΔM15 hsdR lacX74 recA endA tonA | |
| pGM1190 | temperature sensitive plasmid, tsr, aac(3)IV, | (3) |
| | oriT, to terminator PtipA, RBS, fd terminator | |
| pGM1190- | pGM1190 with sgRNA scaffold | This study |
| sgRNA | | |
| pCRISPR- | pGM1190-sgRNA with cas9 | This study |
| Cas9 | | |
| pCRISPR- | pGM1190-sgRNA with dcas9 (D10A and | This study |
| dCas9 | H840A) | |
| pCRISPR- | pCRISPR-Cas9 with a ScaligD expression cassette | This study |
| Cas9-ScaligD | | |
| pCRISPR- | pCRISPR-Cas9 carrying sgRNA: Actlorf1-1 NT | This study |
| Cas9-orf1-1 | | |
| pCRISPR- | pCRISPR-Cas9 carrying sgRNA: Actlorf1-2 T | This study |
| Cas9-orf1-2 | | |
| pCRISPR- | pCRISPR-Cas9 carrying sgRNA: Actlorf1-3 T | This study |
| Cas9-orf1-3 | | |
| pCRISPR- | pCRISPR-Cas9 carrying sgRNA: Actlorf1-4 T | This study |
| Cas9-orf1-4 | | |
| pCRISPR- | pCRISPR-Cas9 carrying sgRNA: Actlorf1-5 T | This study |
| Cas9-orf1-5 | | |
| pCRISPR- | pCRISPR-Cas9 carrying sgRNA: Actlorf1-6 T | This study |
| Cas9-orf1-6 | | |
| pCRISPR- | pCRISPR-Cas9 carrying sgRNA: Actvb-1 NT | This study |
| Cas9-vb1 | | |
| pCRISPR- | pCRISPR-Cas9 carrying sgRNA: Actvb-2 NT | This study |
| Cas9-vb2 | | |
| pCRISPR- | pCRISPR-Cas9 carrying sgRNA: Actvb-3 T | This study |
| Cas9-vb3 | | |
| pCRISPR- | pCRISPR-Cas9 carrying sgRNA: Actvb-4 T | This study |
| Cas9-vb4 | | |
| pCRISPR- | pCRISPR-Cas9 carrying sgRNA: Actvb-5 NT | This study |
| Cas9-vb5 | | |
| pCRISPR- | pCRISPR-Cas9 carrying sgRNA: Actvb-6 NT | This study |
| Cas9-vb6 | | |
| pCRISPR- | pCRISPR-Cas9-orf1-6 with actlORF1 homologous | This study |
| Cas9-orf1-6- | recombination template | |
| Tem | | |
| pCRISPR- | pCRISPR-Cas9-vb2 with actVB homologous | This study |
| Cas9-vb2-Tem | recombination template | |
| pCRISPR- | pCRISPR-Cas9-ScaligD carrying sgRNA: | This study |
| Cas9-ScaligD- | Actlorf1-6 T | |
| orf1-6T | | |
| pCRISPR- | pCRISPR-Cas9-ScaligD carrying sgRNA: | This study |
| Cas9-ScaligD- | Actvb-2 NT | |
| vb2 | | |
| pCRISPR- | pCRISPR-dCas9 carrying sgRNA: orf1p-S1 T | This study |
| dCas9-1 | | |
| pCRISPR- | pCRISPR-dCas9 carrying sgRNA: orf1p-S3 T | This study |
| dCas9-2 | | |
| pCRISPR- | pCRISPR-dCas9 carrying sgRNA: orf1p-S5 T | This study |
| dCas9-3 | | |
| pCRISPR- | pCRISPR-dCas9 carrying sgRNA: orf1p-A1 NT | This study |
| dCas9-4 | | |
| pCRISPR- | pCRISPR-dCas9 carrying sgRNA: orf1p-A4 NT | This study |
| dCas9-5 | | |
| pCRISPR- | pCRISPR-dCas9 carrying sgRNA: orf1p-A5 NT | This study |
| dCas9-6 | | |
| pCRISPR- | pCRISPR-dCas9 carrying sgRNA: Actlorf1-1NT | This study |
| dCas9-7 | | |
| pCRISPR- | pCRISPR-dCas9 carrying sgRNA: Actlorf1-2T | This study |
| dCas9-8 | | |
| pCRISPR- | pCRISPR-dCas9 carrying sgRNA: Actlorf1-3T | This study |
| dCas9-9 | | |
| pCRISPR- | pCRISPR-dCas9 carrying sgRNA: Actlorf1-4T | This study |
| dCas9-10 | | |
| pCRISPR- | pCRISPR-dCas9 carrying sgRNA: Actlorf1-7NT | This study |
| dCas9-11 | | |
| pCRISPR- | pCRISPR-dCas9 carrying sgRNA: Actlorf1-8NT | This study |
| dCas9-12 | | |

Cas9 Codon Optimization for Streptomycetes

The most studied CRISPR-Cas9 system is from Streptococcus pyogenes. As there is significant difference of GC content (35% vs. 72%) and codon usage between S. pyogenes and Streptomyces coelicolor, a codon optimization of the S. pyogenes cas9 according to the codon usage of streptomycetes was performed. In order to make the optimized cas9 as compatible as possible for all streptomycetes, the codon usage table of the most studied actinomycete, Streptomyces coelicolor was used as template for codon optimization, using the S. pyogenes cas9 sequence as starting sequence (SEQ ID NO: 3).

The codon optimization was done by GenScript inc. using the OptimumGene™ algorithm, which optimizes a variety of parameters critical to the efficiency of gene expression, including but not limited to: codon usage bias, GC content, CpG dinucleotides content, mRNA secondary structure, cryptic splicing sites, premature PolyA sites, internal chi sites and ribosomal binding sites, negative CpG islands, RNA instability motif (ARE), repeat sequences (direct repeat, reverse repeat, and Dyad repeat) and restriction sites that may interfere with cloning.

The S. pyogenes cas9 gene comprises tandem rare codons that can reduce the efficiency of translation or even disengage the translational machinery. The codon usage bias in Streptomyces coelicolor was modified by upgrading the CAI from 0.09 to 0.94. GC content (from 35.04 to 61.79) and unfavorable peaks were optimized to prolong the half-life of the mRNA. The Stem-Loop structures, which impact ribosomal binding and stability of mRNA, were broken. In addition, negative cis-acting sites were screened and successfully modified.

Design of the sgRNA Scaffold

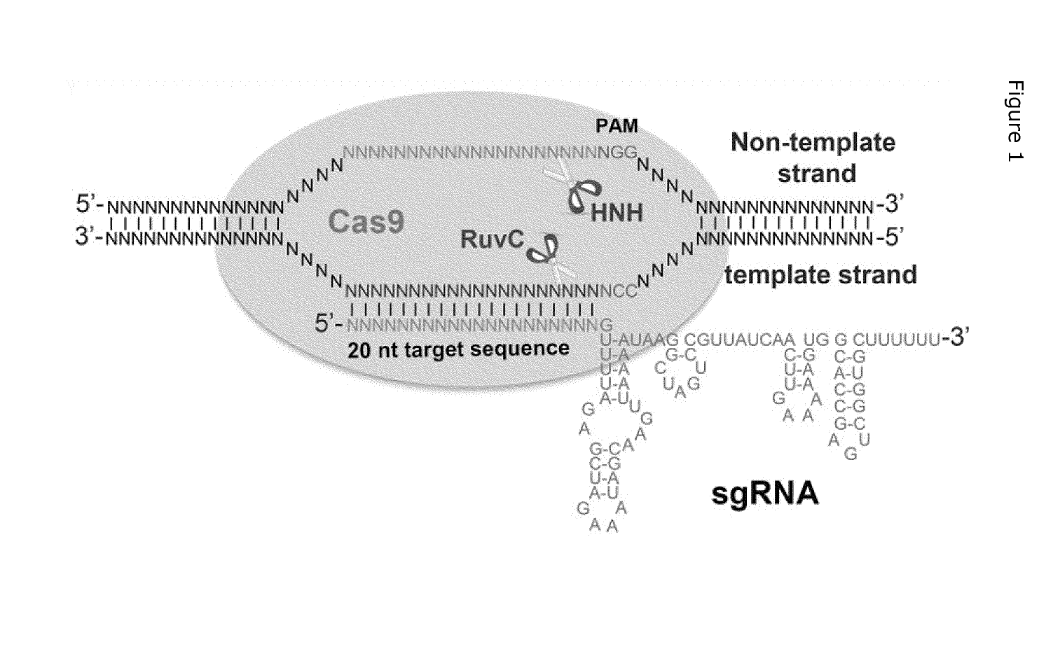

The sequence of the core guide RNA is GTTTTAGAGCTAGAAATAGCAAGTTAAAATAAGGCTAGTCCGTTATCAACTTGAAAAAGTGGCACCGAGTCGGTGCTTTTTT (SEQ ID NO: 67); the RNA structure is shown in FIG. 1. An ermE* promoter was introduced upstream the core sequence and two unique restriction sites, NcoI and SnaBI (underlined) were introduced into the scaffoled in order to make the scaffold easy adaptable when changing the 20 nt target sequences. When constructing new functional sgRNAs, only the 20 nt target sequence of the forward primer needs be changed, while the reverse primer including the SnaBI restriction site needs not be changed.

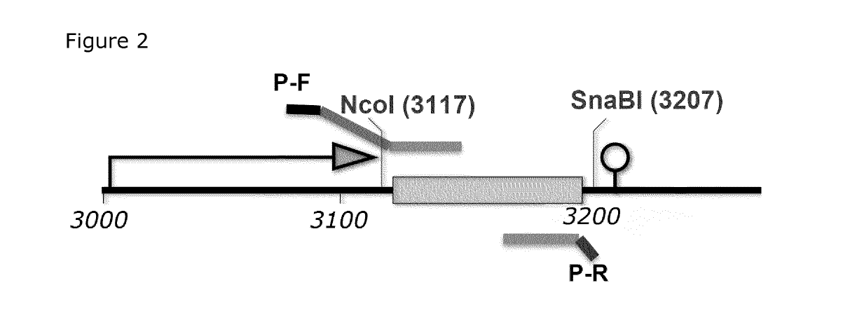

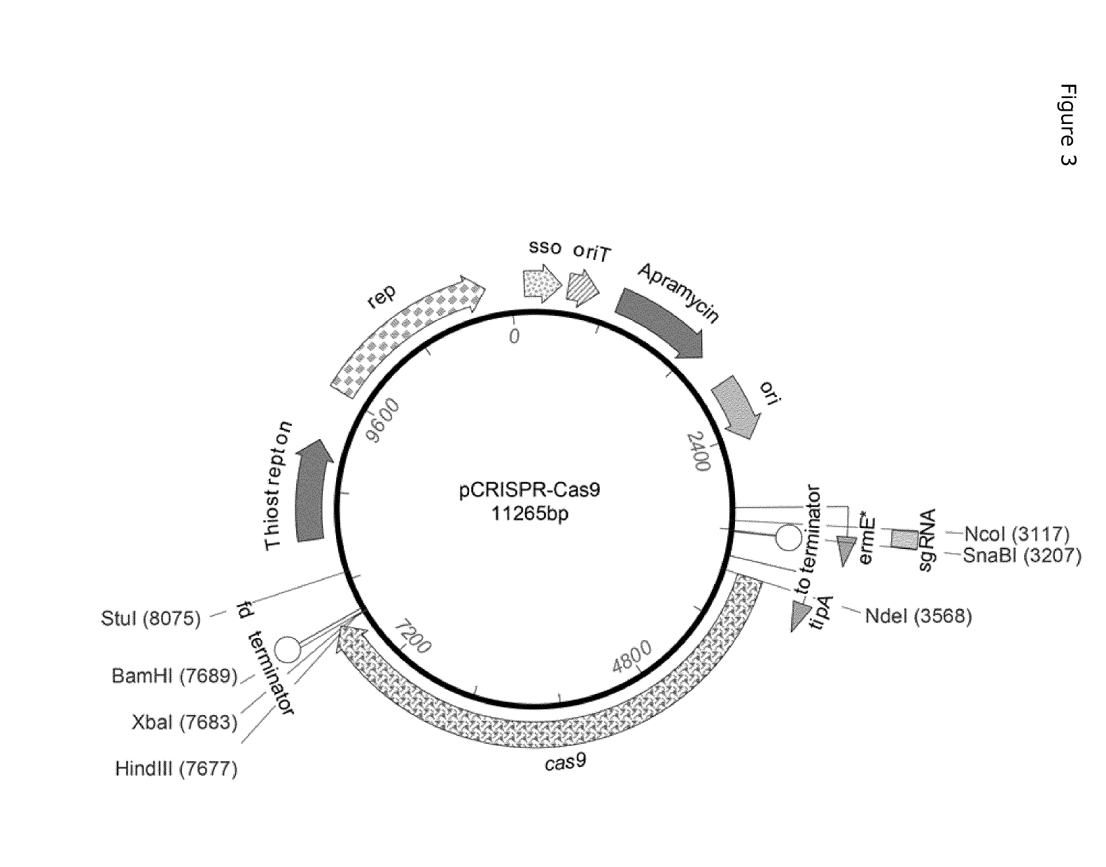

The fragment is amplified by PCR and digested using the NcoI and SnaBI sites before cloning the functional sgRNA into the vector, under the control of the ermE* promotor (FIG. 2). The final sgRNA scaffold sequence is:

(SEQ ID NO: 68)
GCGGTCGATCTTGACGGCTGGCGAGAGGTGCGGGGAGGATCTGACCGAC-
GCGGTCCACACGTGGCACCGCGATGCTGTTGTGGGCACAATCGTGCCGGT
TGG-TAGGATCGAC-
GGCCATGG(N₂₀)GTTTTAGAGCTAGAAATAGCAAGTTAAAATAAGGCTA
GTCCGTTATCAACTTGAAAAAGTGGCACCGAGTCGGTGCTTTTTTTACGT
A,

where N₂₀ represents the 20 nt target sequence.

For the “one plasmid strategy”, we selected the vector pGM1190 (Muth et al., 1989) as the backbone. pGM1190 is temperature sensitive in streptomycetes and will be lost at temperatures above 34° C.; the selection markers are apramycin and thiostrepton, the regulatory elements include: a thiostrepton-inducible promoter tipA, a RBS, a to and an fd terminator. This plasmid can be shuttled in E. coli and streptomycetes.

The sgRNA scaffold was subcloned into pGM1190 upstream of the to terminator using the Gibson cloning method, resulting in pGM1190-sgRNA. The to terminator exited in pGM1190 is used as a secondary terminator for the sgRNA scaffold. Alternatively, it can be sub-cloned into a different vector; this strategy is termed the ‘two plasmids strategy’.

Construction of One Plasmid Based CRISPR-Cas9 System

The codon optimized Cas9 was synthetized as set forth in SEQ ID NO: 1, flanked by the following restriction sites: CATATG in the 5′-end, where ATG is the start codon of SEQ ID NO: 1; and AAGCTTTCTAGA in the 3′-end, immediately downstream of the stop codon.

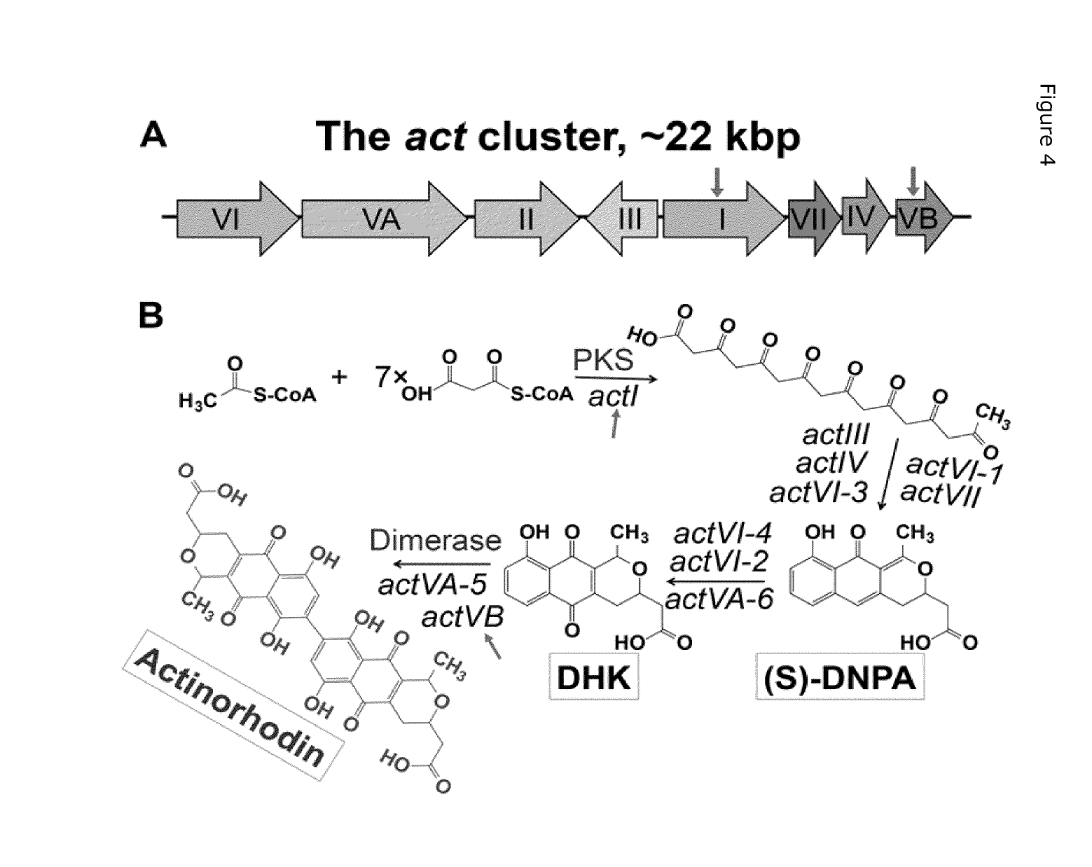

For the one plasmid strategy, the gene was sub-cloned into pGM1190-sgRNA with NdeI and XbaI sites, under the control of the thiostrepton inducible tipA promoter. The final vector was named pCRISPR-Cas9 (FIG. 3). The sgRNA and cas9 fragments were confirmed by PCR (with the primers, sgRNA check-F and sgRNA check-R) and digested by NdeI and XbaI.

Insertion of the Target Sequence Into the Guide RNA

In order to construct a functional vector for the one plasmid strategy, it is sufficient to introduce the 20 nt target sequence upstream of the sgRNA. Design software such as CRISPRy and other similar software can be used for sgRNA design. Here, we used CRISPRy for S. coelicolor (http://staff.biosustain.dtu.dk/laeb/crispy_scoeli/or, or http://crispy.secondarymetabolites.org).

Based on the specificity of the target sequences with the gene, one or more target sequences were chosen. Based on the target sequences, the forward PCR primer as designed: CATGCCATGG N₂₀GTTTTAGAGCTAGAAATAGC (N₂₀ is the 20 nt target sequence) (SEQ ID NO: 69), while the reverse primer remains the same: ACGCCTACGTAAAAAAAGCACCGACTCGGTGCC (sgRNA-R; SEQ ID NO: 44) (the restriction sites are underlined). PCR as used to amplify the functional sgRNAs from the pCRISPR-Cas9 template. The PCR products were digested with NcoI and SnaBI. The pCRISPR-Cas9 was also digested with the same restriction enzymes. After agrose gel purification, the ˜110 bp PCR fragment and the ˜11 kb pCRISPR-Cas9 backbone were ligated by T4 ligase and the ligation mix was transformed into competent E. coli. Several positive transformants for each target sequence were picked for colony PCR screening using the primers, sgRNA check-F and sgRNA check-R. The expected sizes were 234 bp for positive clones and were confirmed by sequencing.

### Example 2

### Generation of Random-Sized Deletions Around a Target Site

This example describes how to apply the present method to inactivate the actinorhdin biosynthetic genes, as well as control the target gene expression in Streptomyces coelicolor A3(2). S. coelicolor A3(2) is a well-known actinorhdin producer. Actinorhodin is a benzoisochromanequinone polyketide antibiotic with pH-dependent colors: blue color when pH>7, red color when pH<7.

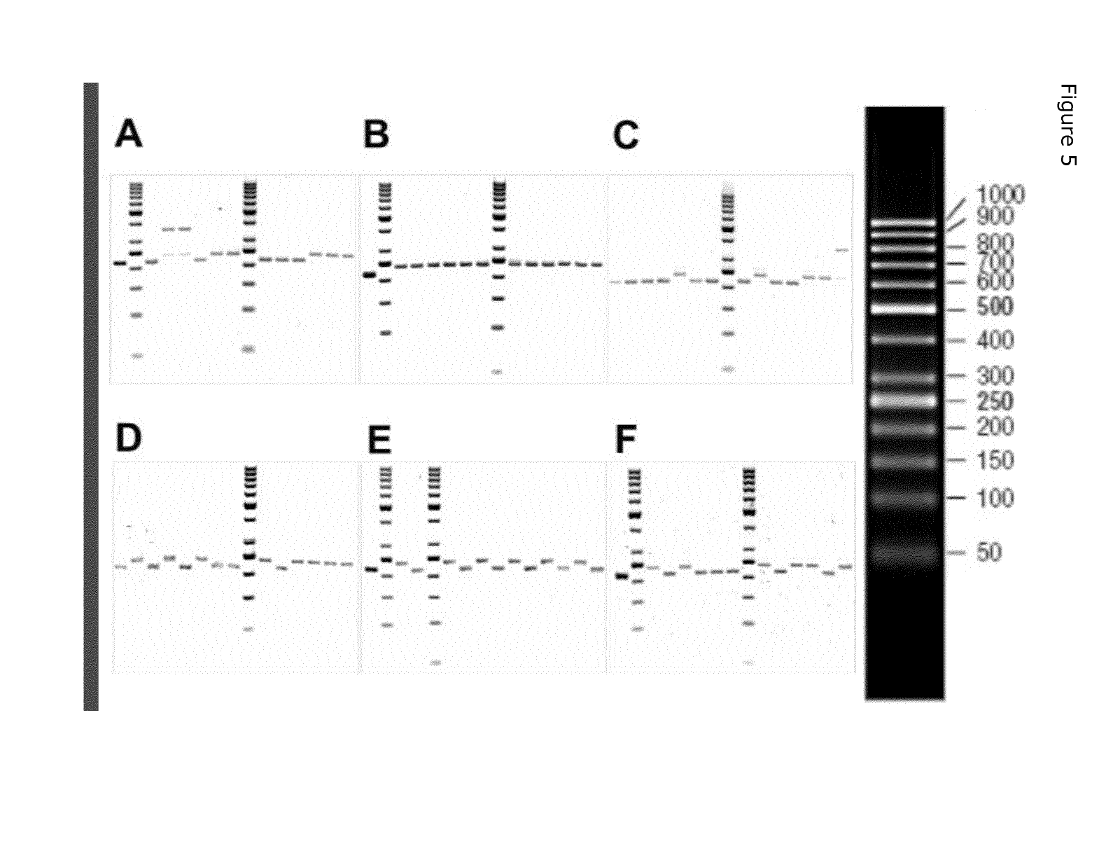

Actinorhdin biosynthesis is encoded by a PKS type II gene cluster, named act gene cluster (FIG. 4). The steps to synthetize actinorhodin are: I. 1× Acetyl-CoA and 7× malonyl-CoA are condensed to form the carbon skeleton by ActI; II. The above carbon backbone is cyclized to form a three ring intermediate, DNPA by ActIII, ActVII, ActIV, ActVI-1 and ActVI-3; III. DNPA is then modified to form DHK by ActVI-2, ActVI-4 and ActVA-6; IV. 2 DHK is dimerized to form the final product, actinorhodin, by ActVA-5 and ActVB (FIG. 4). Two genes were selected as targets (marked by arrows in FIG. 4):

ActORF1 is the actinorhodin ketosynthase subunit alpha (KS domain of PKS II), and ActVB is the actinorhodin polyketide dimerase. A deletion of any of these two genes results in a loss of actinorhodin production, which can be easily monitored by the disappearance of the blue pigment.

For each gene inactivation, 6 different sgRNAs were designed for each gene using CRISPRy webserver (http://staff.biosustain.dtu.dk/laeb/crispy_scoeli/), resulting in 12 sgRNAs (listed in Table 3).

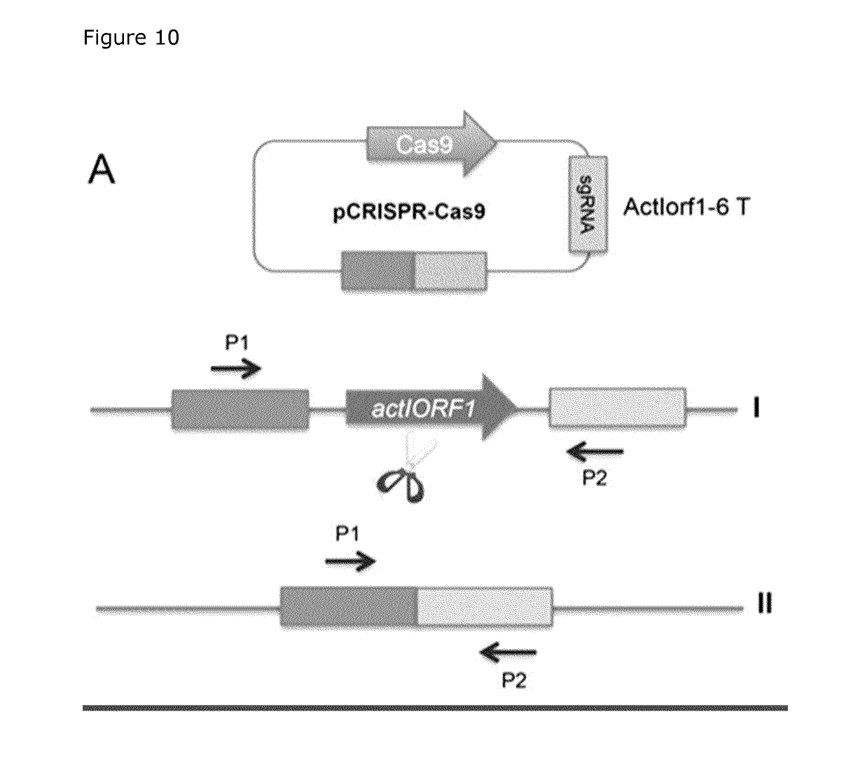

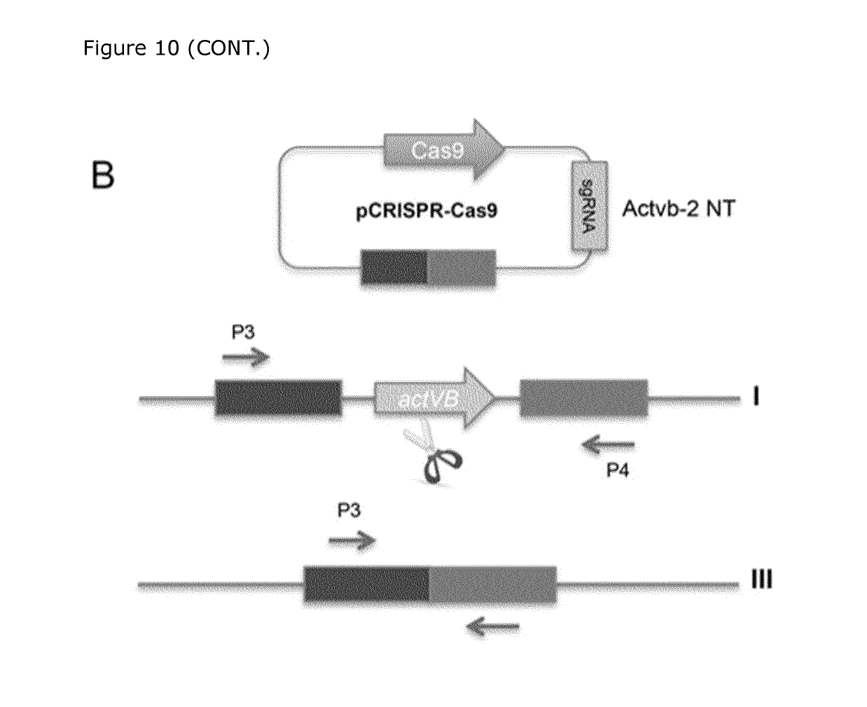

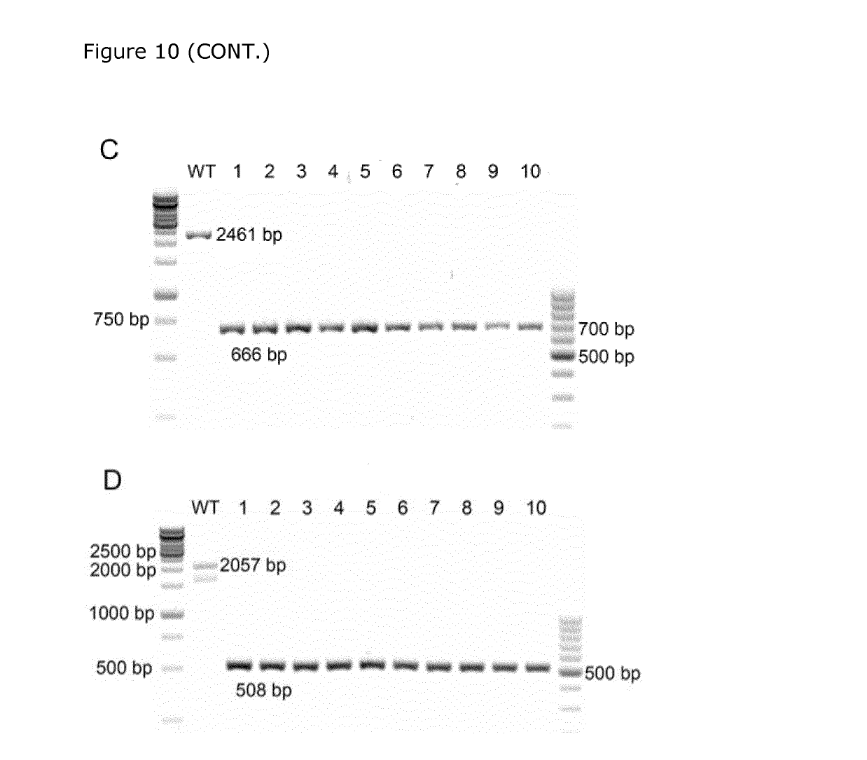

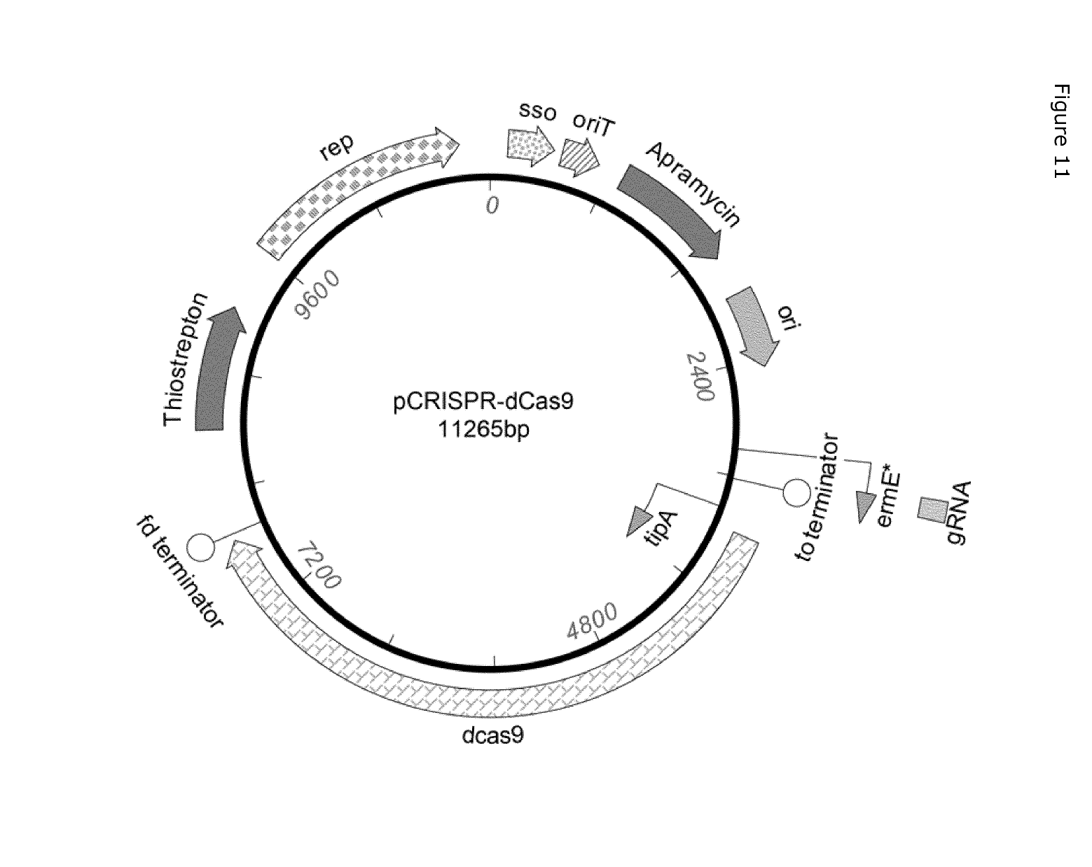

PCR was used to amplify the functional sgRNAs from the pCRISPR-Cas9 template (for primers, see Table 4). The fragments and pCRISPR-Cas9 were digested using NcoI and SnaBI. After agarose gel purification, the PCR fragment (1-10 bp) and the pCRISPR-Cas9 backbone (˜11 kb) were ligated, and transferred into One Shot® Mach1™-T1^{R} chemically competent E. coli. 6 positive transformants for each target sequence were picked for colony PCR screening using the primers set, sgRNA check-F and sgRNA check-R (Table 4), a set of primers resulting in products of 234 bp for positive clones and 214 bp for the negative clones. The PCR screening results are shown in FIG. 10A-F (A-C for actlORF1, D-F for actVB).

2-3 positive clones for each target sequence were confirmed by sequencing and matched the results of the colony PCR 100%. Colony PCR is thus a valid way of screening the clones.

One correct clone for each target sequences was selected randomly to be transferred into the ET12567/pUZ8002 E. coli strain for conjugation. In addition, two negative controls were used: the first is the empty vector, pCRISPR-Cas9 (No Target), which has no target matches on the genome, and the second is a target sequence with a 3 nt PAM motif “NGG”. The inclusion of the PAM as part of the sgRNA abolishes correct recognition of the genomic target (Mismatch).

The PCR validated conjugates for each target sequence plus the two controls were inoculated into 20 ml LB broth with 25 μg/ml kanamycin, 25 μg/ml chloramphenicol and 50 μg/ml apramycin. After overnight shaking at 37° C., the E. coli cells were harvested by centrifuging at 5000 g for 5 minutes at room temperature; fresh LB was used without antibiotics to wash 2 times. The donor cells then were resuspended in 0.5-2 ml LB broth and placed at room temperature. To collect S. coelicolor, spores from one ISP2 plate were resuspended in 0.9% saline, and filtered through a cotton pad. The spore suspension was concentrated by centrifuging at 5000 g for 5 minutes at room temperature, then the spores were resuspended in 0.5 ml-1 m1 2×YT broth. To induce germination, the spore suspension was heated to 50° C. for 10 minutes, and then cooled down to room temperature. 500 μl of the relevant ET12567/pUZ8002 cells were added to the heat treated pre-germinated spores and mixed by inversion. The mixture was centrifuged for 2 minutes at top speed, the supernatant was decanted and the pellet was resuspended in the remaining fluid so that the final volume was about 50 μl. The cells were then plated on Cullum agar plates and incubated for 16 h at 30° C. After 16 h, the plates were overlaid with a solution containing the selection antibiotics: 20 μl of 50 mg/ml nalidixic acid, against E. coli cells or 10 μl of 100 mg/ml apramycin for the selection of clones with the transferred DNA, dissolved in 1 ml of sterile H₂O. The overlaid plates were further incubated for 3-7 days at 30° C., or until colonies became visible. 50-80 conjugates for each target sequence were randomly picked onto ISP2 plates with 50 μg/ml apramycin, 50 μg/ml nalidixic acid (to avoid E. coli contamination), and 1 μg/ml thiostrepton (to induce Cas9). In parallel, the same sets of clones were also streaked onto ISP2 plate with 50 μg/ml apramycin and 50 μg/ml nalidixic acid, but without thiostrepton. The plates were incubated for 7-10 days at 30° C.

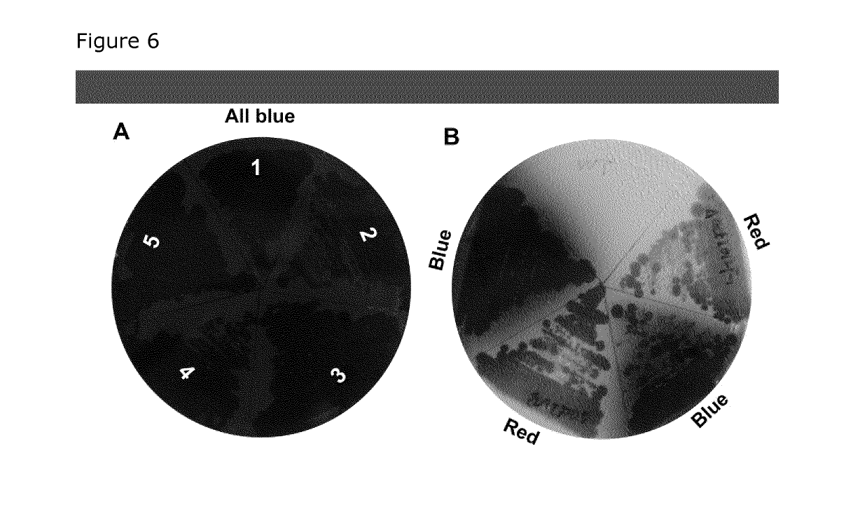

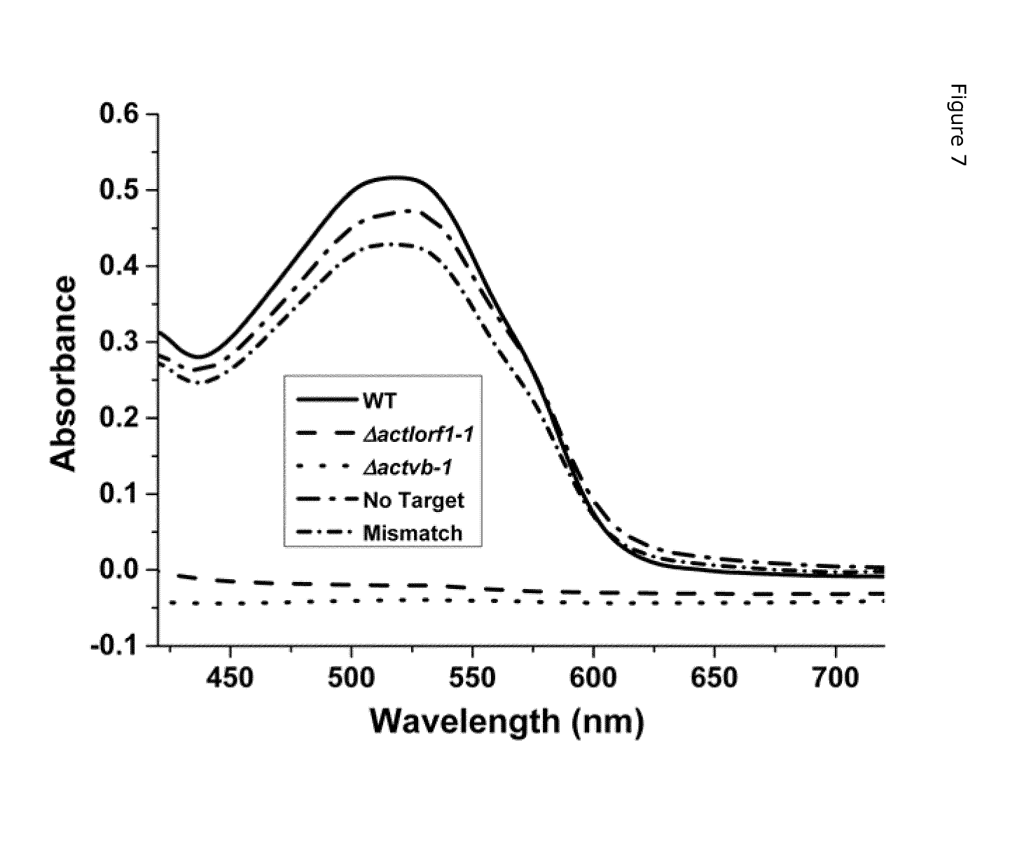

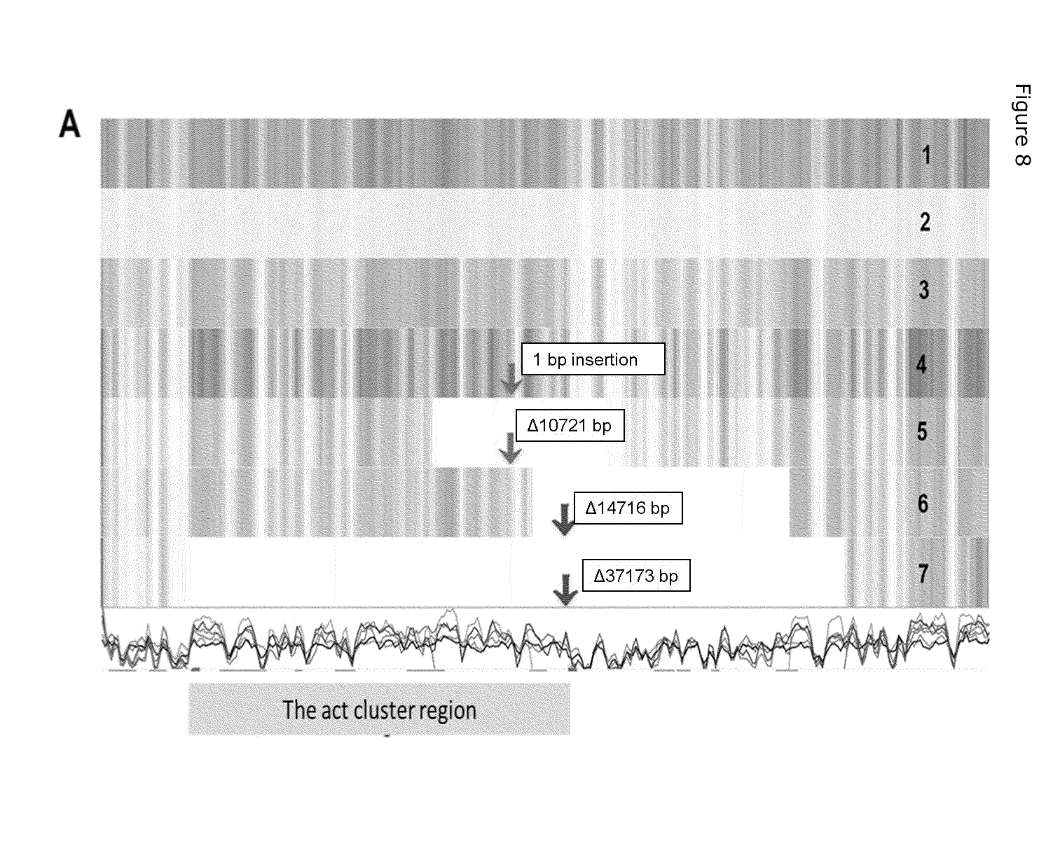

From the red colonies, the following clones were randomly selected: one clone for each gene (Δactlorf1-1 and Δactvb-1), as well as one clone for each negative control (Mismatch and No Target), and one clone for the wild type (WT), resulting in 5 strains (FIG. 6 and FIG. 7).

Besides ISP2 agar plates, the above selected five strains (from ISP2 plates with thiostrepton) were also inoculated in 100 ml ISP2 liquid medium, and incubated with shaking for 7 days at 30° C. 30 ml cultures were used for each strain to perform actinorhodin extraction. The cultures were centrifuged at 8000 g for 10 minutes at room temperature, the supernatant was transferred to a 50 ml tube, the pH was adjusted to 2 with 1M HCl, before adding ¼ volume chloroform. The solution was intensively mixed by vortex, and then centrifuged at 8000 g for 5 minutes at room temperature. The chloroform phase was collected for drying, the dried samples were re-dissolved using 2 ml solvent (methanol: chloroform=1:1). The solutions were analyzed using the Evolution™ 201/220 UV-Visible Spectrophotometers to scan from 420 nm to 720 nm (the actinorhodin in these conditions has a maximum absorption at about 530 nm). The scanning results show that the actinorhodin peaks in Δactlorf1-1 and Δactvb-1 disappeared (FIG. 7).

Genomic DNA was extracted using 10 ml of the above cultures for each strain using Blood & Cell Culture DNA Kit (QIAGEN, Germany). The genomic libraries were generated using the TruSeq® Nano DNA LT Sample Preparation Kit (Illumina Inc., San Diego Calif.). Briefly, 100 ng of genomic DNA diluted in 52.5 μl TE buffer was fragmented in Covaris Crimp Cap microtubes on a Covaris E220 ultrasonicator (Covaris, Brighton, UK) with 5% duty factor, 175 W peak incident power, 200 cycles/burst, and 50 s duration under frequency sweeping mode at 5.5 to 6° C. (Illumina recommendations for a 350-bp average fragment size). The ends of fragmented DNA were repaired by T4 DNA polymerase, Klenow DNA polymerase, and T4 polynucleotide kinase. The Klenow exo minus enzyme was then used to add an ‘A’ base to the 3′ end of the DNA fragments. After the ligation of the adapters to the ends of the DNA fragments, DNA fragments ranging from 300-400 bp were recovered by bead purification. Finally, the adapter-modified DNA fragments were enriched by 3 cycle-PCR. The final concentration of each library was measured by Qubit® 2.0 Florometer and Qubit DNA Broad range assay (Life Technologies, Paisley, UK). The average sizes of the dsDNA libraries were determined using the Agilent DNA 7500 kit on an Agilent 2100 Bioanalyzer. Libraries were normalised and pooled in 10 mM Tris-Cl, pH 8.0, plus 0.05% Tween 20 to the final concentration of 10 nM. After denaturation in 0.2N NaOH, a 10 pm pool of 20 libraries in 600 μl ice-cold HT1 buffer was loaded onto the flow cell provided in the MiSeq Reagent kit v2 (300 cycles) and sequenced on a MiSeq (Illumina Inc., San Diego, Calif.) platform with a paired-end protocol and read lengths of 151 nt.

Mapping of the Sequencing Reads to the S. Coelicolor A3(2) Reference Genome (Genbank Accession AL645882).

The reads obtained above were mapped to the S coelicolor A3(2) reference genome using the software BWA (Li et al., 2009) using the BWA-mem algorithm. The data was inspected and visualized using readXplorer (Hilker et al., 2014) and Artemis (Rutherford et al., 2000). Comparison of the refererence S. coelicolor A3(2) wild type strain used in this study with the S. coelicolorA3(2) reference sequence deposited as AL645882 in Genbank resulted in 95 SNPs and fragment (5797650-5818686) deletion. For the following, S. coelicolor A3(2) WT refers to the sequences obtained in this study. The detailed mapping results are shown Table 6.

TABLE 6
List of mutations detected from whole genome sequencing (the results
shown are after subtracted from the WT)

| Name | Position | Mutation | Annotation | Gene | Description |
|---|---|---|---|---|---|
| Mismatch | 2,474,084 | A→C | T8P | SCO2305→ | putative ABC |
| | | | (ACC→CCC) | | transporter |
| | | | | | ATP-binding subunit |
| | 4,477,934 | 2 bp→TC | coding | SCO4084→ | hypothetical protein |
| | | | (195-196/ | | SCD25.20 |
| | | | 609 nt) | | |
| | 8,265,166 | G→C | intergenic | SCO7449→/ | putative membrane |
| | | | (+76/−125) | →SCO7450 | protein./ |
| | | | | | putative secreted |
| | | | | | protein |
| | 8,267,257 | G→C | intergenic | SCO7451→/ | conserved hypothetical |
| | | | (+13/+26) | ←SCO7452 | protein |
| | | | | | SC5C11.08/putative |
| | | | | | O- |
| | | | | | methyltransferase. |
| No Target | 1,645,577 | +G | intergenic (−554/ | SCO1536←/ | conserved hypothetical |
| | | | +422) | ←SCO1537 | protein |
| | | | | | SCL2.26c/putative |
| | | | | | transport system |
| | | | | | membrane |
| | | | | | protein |
| | 1,645,634 | A→G | intergenic (−611/ | SCO1536←/ | conserved hypothetical |
| | | | +365) | ←SCO1537 | protein |
| | | | | | SCL2.26c/putative |
| | | | | | transport system |
| | | | | | membrane |
| | | | | | protein |
| | 2,462,898 | (G)12→13 | intergenic (−386/ | SCO2292←/ | secreted endo- |
| | | | +324) | ←SCO2293 | 1,4-beta-xylanase |
| | | | | | B (xylanase |
| | | | | | B)/putative integral |
| | | | | | membrane |
| | | | | | protein |
| | 5,093,984 | G→C | P550A | SCO4664← | putative integral |
| | | | (CCC→GCC) | | membrane protein |
| | 6,442,710 | (G)9→10 | intergenic (−96/ | SCO5885←/ | putative |
| | | | +43) | ←SCO5886 | membrane |
| | | | | | protein/3-oxoacyl- |
| | | | | | [acyl-carrier- |
| | | | | | protein] synthase |
| | | | | | II |
| | 8,163,408 | T→C | T129T | SCO7350← | putative membrane |
| | | | (ACA→ACG) | | efflux protein. |
| | 2,311,509 | (TGA)4→5 | coding | SCO2148← | cytochrome B |
| | | | (176/1638 | | subunit |
| | | | nt) | | |
| Δactlorf1-1 | 2,440,703 | A→G | L173P | SCO2271← | hypothetical protein |
| | | | (CTC→CCC) | | SCC75A.17c. |
| | 7,846,245 | A→G | S10P | SCO7056← | putative gntR- |
| | | | (TCC→CCC) | | family transcriptional |
| | | | | | regulator |
| | 5,529,858 | .→A | coding | SCO5087← | actinorhodin |
| | | | (58/1404nt) | | polyketide beta- |
| | | | | | ketoacyl synthase |
| | | | | | alpha subunit |
| | 7,846,250 | T→G | D8A | SCO7056← | putative gntR- |
| | | | (GAC→GCC) | | family transcriptional |
| | | | | | regulator |
| Δactlorf1-2 | 2,462,898 | (G)12→11 | intergenic (−386/ | SCO2292←/ | secreted endo- |
| | | | +324) | ←SCO2293 | 1,4-beta-xylanase |
| | | | | | B (xylanase |
| | | | | | B)/putative integral |
| | | | | | membrane |
| | | | | | protein |
| | 7,846,245 | A→G | S10P | SCO7056← | putative gntR- |
| | | | (TCC→CCC) | | family transcriptional |
| | | | | | regulator |
| | 8,267,257 | G→C | intergenic | SCO7451→/ | conserved hypothetical |
| | | | (+13/+26) | ←SCO7452 | protein |
| | | | | | SC5C11.08/putative |
| | | | | | O-methyltransferase. |
| | 5,527,269 | Δ10721 | | [SCO5084]-[SCO5096] | 11 genes lost, |
| | | | | | SCO5087 included |
| Δactvb-2 | 4,501,350 | T→G | T39P | SCO4102← | putative MerR |
| | | | (ACC→CCC) | | family transcriptional |
| | | | | | regulator |
| | 5,500,560 | G→C | intergenic (−152/ | SCO5060←/ | putative integral |
| | | | −34) | →SCO5061 | membrane protein/ |
| | | | | | putative |
| | | | | | ATP/GTP binding |
| | | | | | protein |
| | 5,500,565 | T→C | intergenic (−157/ | SCO5060←/ | putative integral |
| | | | −29) | →SCO5061 | membrane protein/ |
| | | | | | putative |
| | | | | | ATP/GTP binding |
| | | | | | protein |
| | 7,557,356 | G→C | intergenic | SCO6794→/ | putative membrane |
| | | | (+35/−82) | →SCO6795 | protein./ |
| | | | | | conserved |
| | | | | | hypothetical protein |
| | | | | | SC1A2.04. |
| | 7,557,360 | G→C | intergenic | SCO6794→/ | putative membrane |
| | | | (+39/−78) | →SCO6795 | protein/ |
| | | | | | conserved |
| | | | | | hypothetical protein |
| | | | | | SC1A2.04. |
| | 7,959,767 | T→C | T571A | SCO7164← | hypothetical protein |
| | | | (ACC→GCC) | | SC9A4.26c |
| Δactvb-1 | 2,440,703 | A→G | L173P | SCO2271← | hypothetical protein |
| | | | (CTC→CCC) | | SCC75A.17c. |
| | 3,180,456 | A→C | intergenic | SCO2928→/ | putative asnC- |
| | | | (+74/+48) | ←SCO2929 | family transcriptional |
| | | | | | regulator/ |
| | | | | | putative transposase |
| | 5,513,345 | Δ37,173 | | [SCO5070]-[SCO5107] | 38 genes lost, |
| | | bp | | | SCO5092 included |
| sgRNA: | 5,818,673 | Δ1 bp | intergenic | SCO5350→/— | hypothetical protein |
| Actvb-5 | | | (+125/—) | | SCBAC5H2.19/— |
| NT | | | | | |
| | 7,186,210 | Δ9 bp | coding | SCO6492→ | hypothetical protein |
| | | | (1379-1387/ | | |
| | | | 1998 | | |
| | | | nt) | | |
| | 5,532,664 | Δ14,716 | | [SCO5089]-[SCO5105] | 17 genes lost, |
| | | bp | | | SCO5092 included |

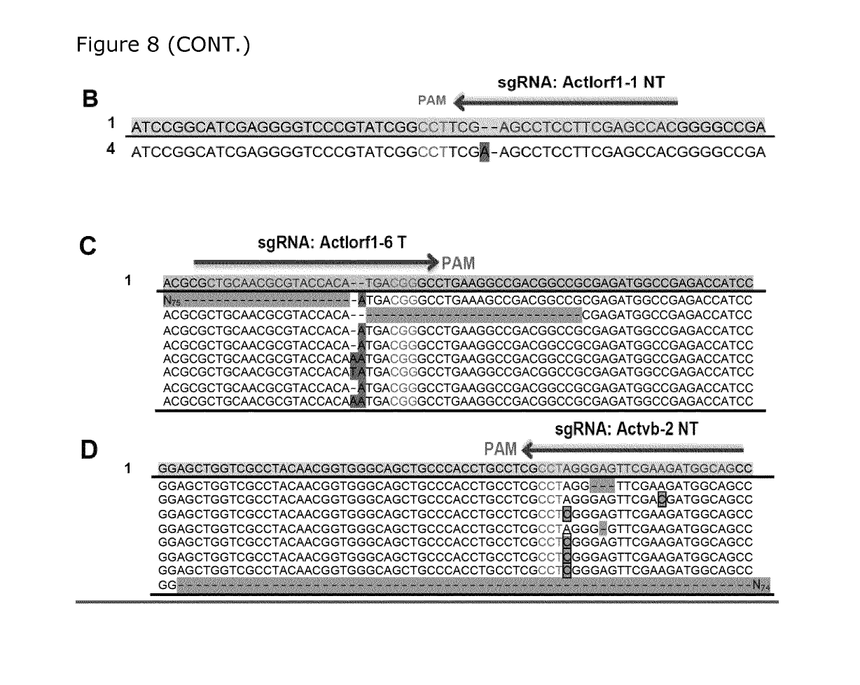

Interestingly, the inactivation of the genes were caused by rearrangement events including 1 bp insertions and deletions between 1 bp and more than 30000 bps around the DSB site (FIG. 8A and B). In other words, the deletion can be both very precise and random sized around the DSB site. It appears this is effect is due to partially deficient NHEJ in S. coelicolor.

It was also tested whether deletions could be generated in other organisms. Deletions were successfully generated in Streptomyces collinus Tü365, in Streptomyces avermitilis, Streptomyces pristinaespiralis and Verrucosispora spp.

Streptomyces collinus TO365 and in Verrucosispora spp. were investigated further , and random-sized deletions ranging from a few kilobase pairs to more than 1 kb were observed.

| | Deletion | Numbers of tested genes |
|---|---|---|
| Species tested | size (kb) | (gene clusters) |
| Streptomyces collinus Tü365 | 23-1200 | 6 |
| Verrucosispora spp. | 5-80 | 3 |

This example shows that the present method can be used to obtain a set of random sized deletions around a precisely defined site from a target sequence in different microorganisms using the present CRISPR-Cas9 system.

### Example 3

### Generation of Precise Deletions Around a Target Site by Introduction of a Functional NHEJ Pathway

Genome mining indicated that the NHEJ pathway of some streptomycetes is not complete because one core component called DNA ligase D is missing. In order to reconstitute the NHEJ pathway of S. coelicolor, homologues of ligD were identified by blasting, using the mycobacterial ligD amino acid sequence as a query. A homologue of ligD was found in S. carneus.

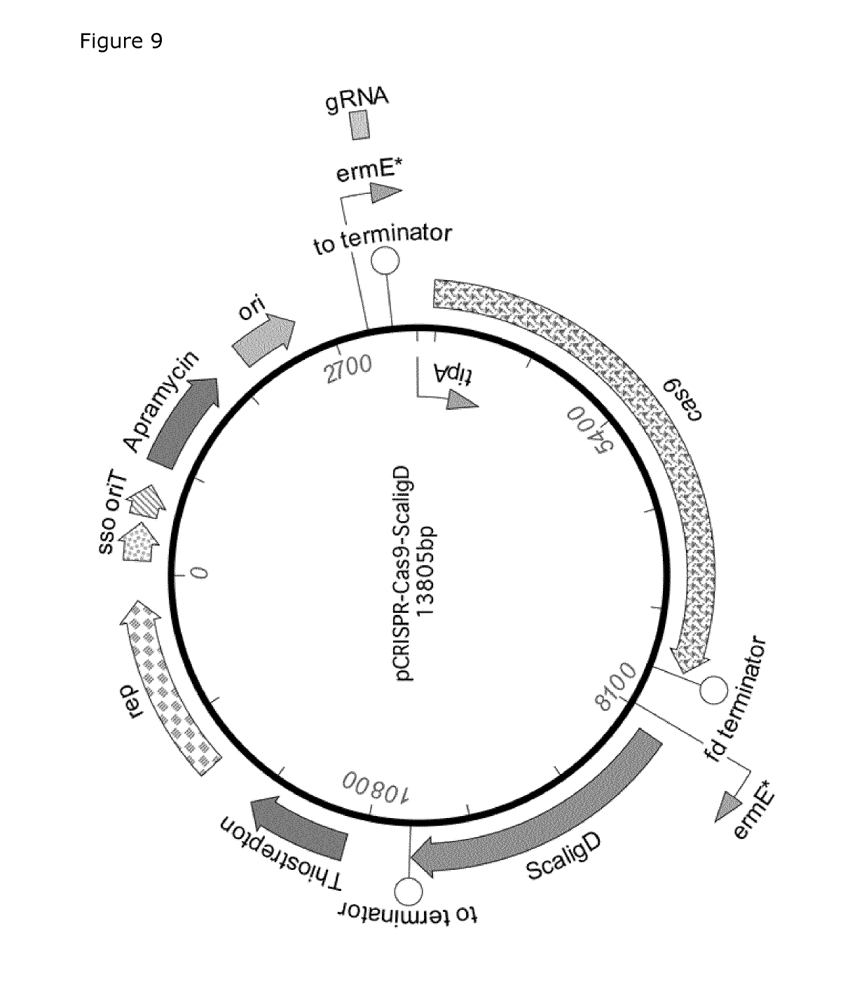

An S. carneus ligD expression cassette was designed, where the S. carneus ligD (ScaligD; SEQ ID NO: 70) was cloned under control of an ermE* promoter, and a to terminator introduced downstream of ligD. This expression cassette was subcloned into the Stul site of pCRISPR-Cas9 by Gibson assembly. The construction was called pCRISPR-Cas9-ligD (FIG. 9).

One sgRNA was selected for each of the two targeted genes (sgRNA: ActIorf1-6 T for actlORF1, and sgRNA: Actvb-2 NT for actVB) to test whether the natively deficient NHEJ pathway was fixed.

Comparison to the non-ScaligD CRISPR-Cas9 system (example 2) showed that the inactivation efficiency increased from 45% to 77%, and 37% to 69% for sgRNA: ActIorf1-6 T and sgRNA: Actvb-2 NT, respectively, after the ScaligD was introduced into the system (Table 7).

TABLE 7
The inactivation efficiency of different sgRNAs
with different DSB repair pathways.

| | | Colony Count^{a} | | | | Efficiency |
|---|---|---|---|---|---|---|
| Ways of | | No | | | | (%) |
| DSB repair | sgRNAs | growth | Red^{b} | Blue | Total | Red/Total |
| Incomplete | Actlorf1-1 | 20 | 31 | 30 | 81 | 38 |
| NHEJ | NT | | | | | |
| | Actlorf1-2 T | 3 | 1 | 7 | 11 | 9 |
| | Actlorf1-3 T | 7 | 18 | 49 | 74 | 24 |
| | Actlorf1-4 T | 43 | 10 | 1 | 54 | 19 |
| | Actlorf1-5 T | 8 | 18 | 8 | 34 | 53 |
| | Actvb-1 | 10 | 20 | 22 | 52 | 38 |
| | NT | | | | | |
| | Actvb-3 T | 17 | 6 | 40 | 63 | 10 |
| | Actvb-4 T | 30 | 6 | 5 | 41 | 15 |
| | Actvb-5 | 7 | 20 | 10 | 37 | 54 |
| | NT | | | | | |
| | Actvb-6 | 1 | 1 | 30 | 32 | 3 |
| | NT | | | | | |
| | Actlorf1-6 T | 10 | 18 | 12 | 40 | 45 |
| | Actvb-2 | 20 | 13 | 2 | 35 | 37 |
| | NT | | | | | |
| Reconstituted | Actlorf1-6 T | 0 | 24 | 7 | 31 | 77 |
| NHEJ | Actvb-2 | 0 | 18 | 8 | 26 | 69 |
| | NT | | | | | |
| HDR (with | Actlorf1-6 T | 0 | 52 | 0 | 52 | 100 |
| homology | Actvb-2 | 0 | 35 | 1 | 36 | 97 |
| templates) | NT | | | | | |

^{a}Denotes the number of colonies with the indicated phenotype after induction with thiostrepton.
^{b}Actinorhodin is blue. Upon loss of actinorhodin production, the red color of the 2^{nd} pigmented antibiotic, undecylprodigiosin, becomes visible.

To further validate this observation, primers were designed to detect the ˜600 bp fragment containing the theoretical cleavage sites of the used sgRNAs. Eight red clones for each gene were randomly selected for colony PCR, and the PCR products were sequenced. No long fragment deletions were found in any of the 16 sequencing clones; instead, most of them just had 1 to 3 bp deletion, substitution, or insertion (FIG. 8C and D). In contrast, without the ScaligD, long fragment deletions were found in 3 of the 4 red clones for which whole genome sequencing was performed (FIG. 8A).

These results indicated the natively deficient incomplete NHEJ pathway was successfully fixed by complementary its missing component, DNA ligase D.

### Example 4

### HDR-Directed Gene Editing

In this example, in order to bypass the NHEJ pathway, a template for homologous recombination was introduced into the CRISPR-Cas9 system to let the organism use HDR to repair the DSBs. Again the genes ActIORF1 and ActVB were selected for testing, only one sgRNA (sgRNA: ActIorf1-6 T, and sgRNA: Actvb-2NT) was designed for each gene. PCR was used to amplify the ˜1 kb fragments of the 5′ and the 3′ regions out of the targeted genes with the primers orf1-5′F, orf1-5′R, orf1-3′F, orf1-3′R, and VB-5′F, VB-5′R, VB-3′F, VB-3′R, for actORF1 and actVB, respectively. The orf1-5′F and VB-5′F primers contain a 20 bp overlap region of the 5′ of the Stul site from the pCRISPR-Cas9 plasmid, and the orf1-3′R and VB-3′R primers contain a 20 bp overlap region of the 3′ of the Stul site from the pCRISPR-Cas9 plasmid, while the orf1-5′R and VB-5′R primers contain a 20 bp overlap region of the orf1-3′ fragment and VB-3′ fragment, respectively. After gel purification of the fragments, orf1-5′, orf1-3′, and the Stul digested pCRISPR-Cas9 plasmid, and VB-5′, VB-3′, and the Stul digested pCRISPR-Cas9 plasmid were assembled by Gibson assembly (New England Biolabs). The transformants were screened by PCR using orf1-check-F, orf1-check-R and VB-check-F, VB-check-R for the homologous recombination templates of actlORF1 and actVB, respectively, and finally confirmed by sequencing. All 52 clones picked randomly for actlORF1, and 35 out of 36 clones picked randomly for actVB were red after induction (Table 7).

In order to find out whether the deletion was a precise deletion, we designed primers around the target cleavage site. For both genes, 10 red clones were randomly selected for colony PCR validation. The colony PCR was performed as follows: mycelia of the selected colonies were scraped from the plates using a sterile toothpick into 10 μl pure DMSO in PCR tubes. The tubes were shaken vigorously for 10 min at 100° C. in a heating block. After this step, the solution was centrifuged at top speed for 10 seconds, 1 μl of the supernatant were used for PCR template in a 20 μl PCR reaction.

The sizes of all 20 PCR products corresponded to the predicted sizes of the gene deletion (FIG. 10). Importantly, the CRISPR-Cas9 system with the homologous recombination template showed even higher efficiency and precision in gene editing in comparison to the gene deletion system relying on functional NHEJ described in example 3 (Table 7).

This example shows that gene editing can be performed in actinomycetes using the CRISPR/Cas9 system with homologous recombination with high precision and efficiency.

### Example 5

### Modulation of Gene Expression

This example describes how gene expression in Actinomycetes can be modulated. The actlORF1 gene was selected for these experiments.

The codon-optimised Cas9 (SEQ ID NO: 1) was mutated to a catalytically dead version, which was done by point mutation of D10A and H840A. This version of Cas9 was called dCas9 and is lacking endonuclease activity (FIG. 11).

Three sgRNAs targeting the non-template strand DNA and three sgRNAs targeting the template strand DNA of the coding region of actlORF1 gene were selected. Another set of three sgRNAs targeting the template/non-template strand of the promoter region of actlORF1 gene (total 12) were chosen (Table 3). In this example, a catalytically dead Cas9 (dCas9) having both mutations D10A and H840A was used.

The cloning strategy for sgRNA was the same as for the CRISPR-Cas9 system for deletion described above. The conjugates were streaked on the ISP2 agar containing 1 μg/ml thiostrepton (the inducer for dCas9), 50 μg/ml apramycin, and 50 μg/ml nalidixic acid and incubated for 7 days at 30° C.

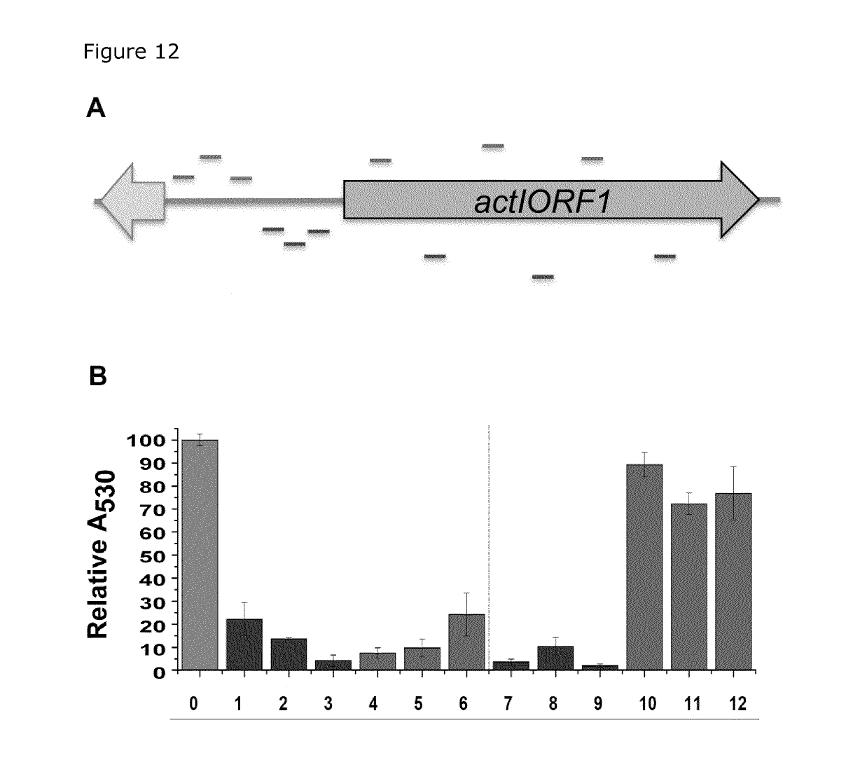

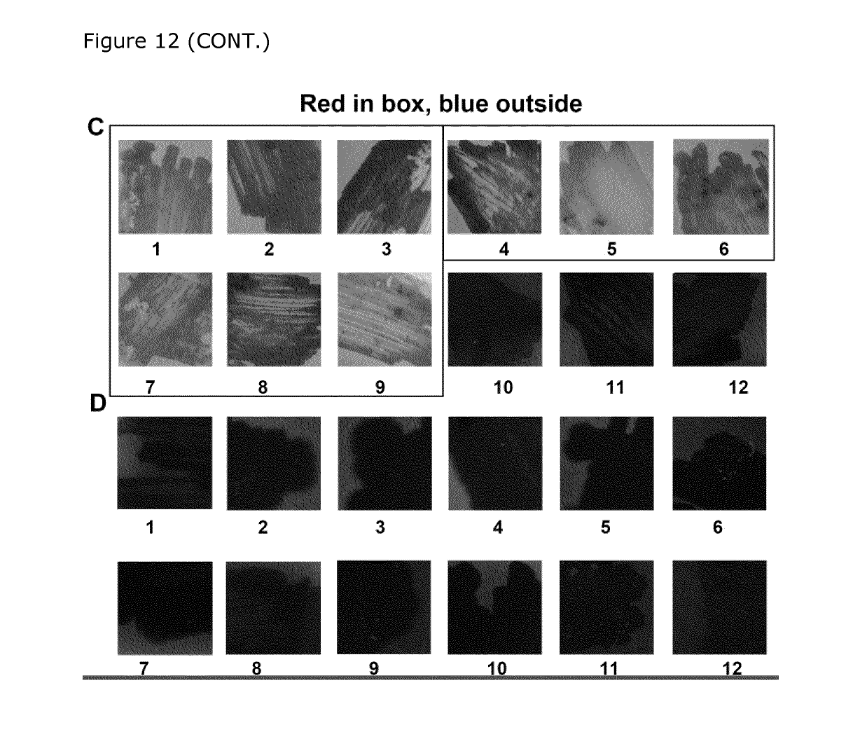

Actinorhodin production was abolished or dramatically reduced (FIG. 12) in clones encoding sgRNAs targeted on the promoter region of actlORF1 gene, independently of which of the template strand DNA or non-template strand DNA was targeted. In contrast, loss or decrease of actinorhodin production in clones carrying sgRNAs that target the coding region, was only observed in the clones with sgRNAs directed to the non-template strand (FIG. 12).

To provoke the loss of the pCRISPR-Cas9 plasmid, the temperature of the incubaton was raised to 37° C. for 24 h, before transferring the cultures to fresh ISP2 plates without antibiotics and incubating for another 5 days at 37° C. The previously red clones began to turn blue (FIG. 12), indicating that the repression of actinorhodin biosynthesis by the CRISPR-dCas9 system was abrogated and the related gene started to express.

This example shows that gene expression can be modulated in actinomycetes by using the present system.

Sequences

| SEQ ID NO | Name | Description |
|---|---|---|
| 1 | Codon-optimised Cas9 | DNA sequence, codon- |
| | | optimised for Streptomyces |
| | | coelicolor |
| 2 | Cas9 protein | Translation of SEQ ID |
| | | NO: 1 |
| 3 | cas9 | DNA from S. pyogenes |
| 4 | Actlorf1-1 NT | Table 3 |
| 5 | Actlorf1-2 T | Table 3 |
| 6 | Actlorf1-3 T | Table 3 |
| 7 | Actlorf1-4 T | Table 3 |
| 8 | Actlorf1-5 T | Table 3 |
| 9 | Actlorf1-6 T | Table 3 |
| 10 | Actvb-1 NT | Table 3 |
| 11 | Actvb-2 NT | Table 3 |
| 12 | Actvb-3 T | Table 3 |
| 13 | Actvb-4 T | Table 3 |
| 14 | Actvb-5 NT | Table 3 |
| 15 | Actvb-6 NT | Table 3 |
| 16 | orf1p-S1 T | Table 3 |
| 17 | orf1p-S3 T | Table 3 |
| 18 | orf1p-S5 T | Table 3 |
| 19 | orf1p-A1 NT | Table 3 |
| 20 | orf1p-A4 NT | Table 3 |
| 21 | orf1p-A5 NT | Table 3 |
| 22 | Actlorf1-7 NT | Table 3 |
| 23 | Actlorf1-8 NT | Table 3 |
| 24 | Actlorf1-F1 | Table 4 |
| 25 | Actlorf1-F2 | Table 4 |
| 26 | Actlorf1-F3 | Table 4 |
| 27 | Actlorf1-F4 | Table 4 |
| 28 | Actlorf1-F5 | Table 4 |
| 29 | Actlorf1-F6 | Table 4 |
| 30 | Actlorf1-F7 | Table 4 |
| 31 | Actlorf1-F8 | Table 4 |
| 32 | ActVB-F1 | Table 4 |
| 33 | ActVB-F2 | Table 4 |
| 34 | ActVB-F3 | Table 4 |
| 35 | ActVB-F4 | Table 4 |
| 36 | ActVB-F5 | Table 4 |
| 37 | ActVB-F6 | Table 4 |
| 38 | orf1p-S1 T-F | Table 4 |
| 39 | orf1p-S3 T-F | Table 4 |
| 40 | orf1p-S5 T-F | Table 4 |
| 41 | orf1p-A1 NT-F | Table 4 |
| 42 | orf1p-A4 NT-F | Table 4 |
| 43 | orf1p-A5 NT-F | Table 4 |
| 44 | sgRNA-R | Table 4 |
| 45 | gRNA check-F | Table 4 |
| 46 | gRNA check-R | Table 4 |
| 47 | orf1-5′F | Table 4 |
| 48 | orf1-5′R | Table 4 |
| 49 | orf1-3′F | Table 4 |
| 50 | orf1-3′R | Table 4 |
| 51 | VB-5′F | Table 4 |
| 52 | VB-5′R | Table 4 |
| 53 | VB-3′F | Table 4 |
| 54 | VB-3′R | Table 4 |
| 55 | VB-check-F | Table 4 |
| 56 | VB-check-R | Table 4 |
| 57 | ORF1-check-F | Table 4 |
| 58 | ORF1-check-R | Table 4 |
| 59 | CAS9-check-F | Table 4 |
| 60 | CAS9-check-R | Table 4 |
| 61 | ScaligD-F | Table 4 |
| 62 | ScaligD-R | Table 4 |
| 63 | orf1-6 ligD test-F | Table 4 |
| 64 | orf1-6 ligD test-R | Table 4 |
| 65 | vb2 ligD test-F | Table 4 |
| 66 | vb2 ligD test-R | Table 4 |
| 67 | core guide RNA | Example 1 |
| 68 | sgRNA scaffold | Example 1 |
| 69 | Target-specific Fw primer | Table 3 |
| 70 | Translation of SEQ ID NO: 3 | |
| 71 | S. carneus ligD DNA | |
| 72 | Translation of SEQ ID NO: | |
| | 71 | |

Codon-optimised Cas9
SEQ ID NO: 1
ATGGACAAGAAGTACTCCATCGGCCTCGACATCGGCACCAACTCCGTGGG
CTGGGCGGTCATCACCGACGAGTACAAGGTCCCCTCCAAGAAGTTCAAGG
TCCTGGGCAACACCGACCGGCACTCGATCAAGAAGAACCTGATCGGCGCC
CTGCTCTTCGACAGCGGCGAGACCGCCGAGGCGACCCGCCTGAAGCGGAC
CGCGCGTCGCCGCTACACCCGGCGCAAGAACCGCATCTGCTACCTGCAGG
AAATCTTCTCCAACGAGATGGCCAAGGTGGACGACTCGTTCTTCCACCGC
CTGGAGGAGAGCTTCCTGGTGGAGGAGGACAAGAAGCACGAGCGCCACCC
GATCTTCGGCAACATCGTGGACGAGGTGGCCTACCACGAGAAGTACCCCA
CCATCTACCACCTCCGCAAGAAGCTGGTGGACTCGACCGACAAGGCGGAC
CTGCGGCTCATCTACCTGGCCCTCGCGCACATGATCAAGTTCCGCGGCCA
CTTCCTCATCGAGGGCGACCTGAACCCGGACAACTCCGACGTGGACAAGC
TCTTCATCCAGCTGGTGCAGACCTACAACCAGCTGTTCGAGGAGAACCCC
ATCAACGCCAGCGGCGTGGACGCCAAGGCGATCCTCTCCGCGCGCCTGGC
AAGTCCCGGCGCCTGGAGAACCTCATCGCCCAGCTGCCGGGCGAGAAGAA
GAACGGCCTCTTCGGCAACCTGATCGCGCTGTCGCTCGGCCTGACCCCCA
ACTTCAAGAGCAACTTCGACCTGGCCGAGGACGCGAAGCTCCAGCTGTCC
AAGGACACCTACGACGACGACCTGGACAACCTGCTCGCCCAGATCGGCGA
CCAGTACGCGGACCTCTTCCTGGCCGCGAAGAACCTCTCGGACGCCATCC
TGCTCAGCGACATCCTGCGGGTCAACACCGAGATCACCAAGGCCCCGCTG
TCGGCGAGCATGATCAAGCGGTACGACGAGCACCACCAGGACCTGACCCT
GCTCAAGGCCCTCGTGCGCCAGCAGCTGCCCGAGAAGTACAAGGAAATCT
TCTTCGACCAGTCCAAGAACGGCTACGCCGGCTACATCGACGGCGGCGCG
TCGCAGGAGGAGTTCTACAAGTTCATCAAGCCGATCCTGGAGAAGATGGA
CGGCACCGAGGAGCTGCTCGTCAAGCTGAACCGCGAGGACCTGCTCCGCA
AGCAGCGGACCTTCGACAACGGCTCCATCCCGCACCAGATCCACCTGGGC
GAGCTCCACGCCATCCTCCGGCGCCAGGAGGACTTCTACCCCTTCTGAAG
GACAACCGCGAGAAGATCGAGAAGATCCTGACCTTCCGCATCCCGTACTA
CGTCGGCCCCCTGGCCCGCGGCAACTCCCGGTTCGCGTGGATGACCCGGA
AGTCGGAGGAGACCATCACCCCGTGGAACTTCGAGGAGGTCGTGGACAAG
GGCGCGTCCGCGCAGTCGTTCATCGAGCGCATGACCAACTTCGACAAGAA
CCTCCCGAACGAGAAGGTCCTGCCCAAGCACTCCCTGCTCTACGAGTACT
TCACCGTGTACAACGAGCTGACCAAGGTCAAGTACGTGACCGAGGGCATG
CGGAAGCCGGCCTTCCTGTCGGGCGAGCAGAAGAAGGCGATCGTGGACCT
GCTCTTCAAGACCAACCGCAAGGTCACCGTGAAGCAGCTGAAGGAGGACT
ACTTCAAGAAGATCGAGTGCTTCGACTCCGTCGAGATCAGCGGCGTGGAG
GACCGCTTCAACGCCTCCCTGGGCACCTACCACGACCTGCTCAAGATCAT
CAAGGACAAGGACTTCCTCGACAACGAGGAGAACGAGGACATCCTGGAGG
ACATCGTCCTCACCCTGACCCTCTTCGAGGACCGCGAGATGATCGAGGAG
CGGCTCAAGACCTACGCCCACCTGTTCGACGACAAGGTGATGAAGCAGCT
GAAGCGTCGCCGCTACACCGGCTGGGGCCGCCTCTCCCGGAAGCTGATCA
ACGGCATCCGGGACAAGCAGAGCGGCAAGACCATCCTGGACTTCCTCAAG
TCCGACGGCTTCGCCAACCGCAACTTCATGCAGCTCATCCACGACGACAG
CCTGACCTTCAAGGAGGACATCCAGAAGGCCCAGGTCTCGGGCCAGGGCG
ACAGCCTCCACGAGCACATCGCCAACCTGGCGGGCTCCCCGGCGATCAAG
AAGGGCATCCTCCAGACCGTCAAGGTCGTGGACGAGCTGGTCAAGGTGAT
GGGCCGCCACAAGCCCGAGAACATCGTGATCGAGATGGCCCGGGAGAACC
AGACCACCCAGAAGGGCCAGAAGAACTCGCGCGAGCGGATGAAGCGGATC
GAGGAGGGCATCAAGGAGCTCGGCAGCCAGATCCTGAAGGAGCACCCGGT
CGAGAACACCCAGCTGCAGAACGAGAAGCTGTACCTCTACTACCTGCAGA
ACGGCCGCGACATGTACGTGGACCAGGAGCTCGACATCAACCGGCTGTCC
GACTACGACGTGGACCACATCGTGCCGCAGTCCTTCCTGAAGGACGACTC
GATCGACAACAAGGTCCTGACCCGCTCGGACAAGAACCGGGGCAAGTCCG
ACAACGTGCCCTCGGAGGAGGTCGTGAAGAAGATGAAGAACTACTGCGCC
AGCTGCTCAACGCCAAGCTCATCACCCAGCGCAAGTTCGACAACCTGACC
AAGGCCGAGCGGGGCGGCCTGAGCGAGCTCGACAAGGCGGGCTTCATCAA
GCGCCAGCTGGTCGAGACCCGGCAGATCACCAAGCACGTGGCCCAGATCC
TGGACTCCCGGATGAACACCAAGTACGACGAGAACGACAAGCTGATCCGC
GAGGTCAAGGTGATCACCCTCAAGAGCAAGCTGGTCTCCGACTTCCGCAA
GGACTTCCAGTTCTACAAGGTCCGGGAGATCAACAACTACCACCACGCCC
ACGACGCGTACCTGAACGCCGTCGTGGGCACCGCGCTGATCAAGAAGTAC
CCGAAGCTGGAGTCCGAGTTCGTCTACGGCGACTACAAGGTCTACGACGT
GCGCAAGATGATCGCCAAGAGCGAGCAGGAGATCGGCAAGGCCACCGCGA
AGTACTTCTTCTACTCCAACATCATGAACTTCTTCAAGACCGAGATCACC
CTGGCCAACGGCGAGATCCGCAAGCGGCCCCTGATCGAGACCAACGGCGA
GACCGGCGAGATCGTCTGGGACAAGGGCCGCGACTTCGCCACCGTCCGGA
AGGTGCTGTCGATGCCGCAGGTCAACATCGTGAAGAAGACCGGGTGCAGA
CCGGCGGCTTCAGCAAGGAGTCCATCCTCCCCAAGCGCAACAGCGACAAG
CTGATCGCCCGGAAGAAGGACTGGGACCCGAAGAAGTACGGCGGCTTCGA
CAGCCCCACCGTCGCCTACTCCGTGCTGGTCGTGGCGAAGGTCGAGAAGG
GCAAGAGCAAGAAGCTGAAGTCCGTGAAGGAGCTGCTCGGCATCACCATC
ATGGAGCGCTCCTCGTTCGAGAAGAACCCGATCGACTTCCTGGAGGCCAA
GGGCTACAAGGAGGTCAAGAAGGACCTCATCATCAAGCTGCCCAAGTACA
GCCTGTTCGAGCTGGAGAACGGCCGCAAGCGGATGCTCGCCTCCGCGGGC
GAGCTGCAGAAGGGCAACGAGCTGGCCCTCCCGTCGAAGTACGTCAACTT
CCTGTACCTCGCGTCCCACTACGAGAAGCTGAAGGGCTCGCCCGAGGACA
ACGAGCAGAAGCAGCTCTTCGTGGAGCAGCACAAGCACTACCTGGACGAG
ATCATCGAGCAGATCAGCGAGTTCAGCAAGCGCGTCATCCTGGCCGACGC
GAACCTCGACAAGGTGCTGTCCGCCTACAACAAGCACCGCGACAAGCCGA
TCCGGGAGCAGGCGGAGAACATCATCCACCTGTTCACCCTCACCAACCTG
GGCGCCCCCGCCGCGTTCAAGTACTTCGACACCACCATCGACCGCAAGCG
GTACACCTCCACCAAGGAGGTCCTCGACGCGACCCTGATCCACCAGAGCA
TCACCGGCCTGTACGAGACCCGCATCGACCTGTCCCAGCTCGGCGGCGAC
TGA
Protein sequence for codon-optimised Cas9:
SEQ ID NO: 2
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKSLLYEYFTVYNELTKVKYVTEGMRKPAFLSGEQKKAIVDLL
FKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMG
RHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIRE
VKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQT
GGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYS
LFELENGRKRMLASAGELQKGNELALPSKYVNFLYLASHYEKLKGSPEDN
EQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSI
TGLYETRIDLSQLGGD.
S. pyogenes cas9
SEQ ID NO: 3
ATGGATAAGAAATACTCAATAGGCTTAGATATCGGCACAAATAGCGTCGGA
TGGGCGGTGATCACTGATGAATATAAGGTTCCGTCTAAAAAGTTCAAGGTT
CTGGGAAATACAGACCGCCACAGTATCAAAAAAAATCTTATAGGGGCTCTT
TTATTTGACAGTGGAGAGACAGCGGAAGCGACTCGTCTCAAACGGACAGCT
CGTAGAAGGTATACACGTCGGAAGAATCGTATTTGTTATCTACAGGAGATT
TTTTCAAATGAGATGGCGAAAGTAGATGATAGTTTCTTTCATCGACTTGAA
GAGTCTTTTTTGGTGGAAGAAGACAAGAAGCATGAACGTCATCCTATTTTT
GGAAATATAGTAGATGAAGTTGCTTATCATGAGAAATATCCAACTATCTAT
CATCTGCGAAAAAAATTGGTAGATTCTACTGATAAAGCGGATTTGCGCTTA
ATCTATTTGGCCTTAGCGCATATGATTAAGTTTCGTGGTCATTTTTTGATT
GAGGGAGATTTAAATCCTGATAATAGTGATGTGGACAAACTATTTATCCAG
TTGGTACAAACCTACAATCAATTATTTGAAGAAAACCCTATTAACGCAAGT
GGAGTAGATGCTAAAGCGATTCTTTCTGCACGATTGAGTAAATCAAGACGA
TTAGAAAATCTCATTGCTCAGCTCCCCGGTGAGAAGAAAAATGGCTTATTT
GGGAATCTCATTGCTTTGTCATTGGGTTTGACCCCTAATTTTAAATCAAAT
TTTGATTTGGCAGAAGATGCTAAATTACAGCTTTCAAAAGATACTTACGAT
GATGATTTAGATAATTTATTGGCGCAAATTGGAGATCAATATGCTGATTTG
TTTTTGGCAGCTAAGAATTTATCAGATGCTATTTTACTTTCAGATATCCTA
AGAGTAAATACTGAAATAACTAAGGCTCCCCTATCAGCTTCAATGATTAAA
CGCTACGATGAACATCATCAAGACTTGACTCTTTTAAAAGCTTTAGTTCGA
CAACAACTTCCAGAAAAGTATAAAGAAATCTTTTTTGATCAATCAAAAAAC
GGATATGCAGGTTATATTGATGGGGGAGCTAGCCAAGAAGAATTTTATAAA
TTTATCAAACCAATTTTAGAAAAAATGGATGGTACTGAGGAATTATTGGTG
AAACTAAATCGTGAAGATTTGCTGCGCAAGCAACGGACCTTTGACAACGGC
TCTATTCCCCATCAAATTCACTTGGGTGAGCTGCATGCTATTTTGAGAAGA
CAAGAAGACTTTTATCCATTTTTAAAAGACAATCGTGAGAAGATTGAAAAA
ATCTTGACTTTTCGAATTCCTTATTATGTTGGTCCATTGGCGCGTGGCAAT
AGTCGTTTTGCATGGATGACTCGGAAGTCTGAAGAAACAATTACCCCATGG
AATTTTGAAGAAGTTGTCGATAAAGGTGCTTCAGCTCAATCATTTATTGAA
CGCATGACAAACTTTGATAAAAATCTTCCAAATGAAAAAGTACTACCAAAA
CATAGTTTGCTTTATGAGTATTTTACGGTTTATAACGAATTGACAAAGGTC
AAATATGTTACTGAAGGAATGCGAAAACCAGCATTTCTTTCAGGTGAACAG
AAGAAAGCCATTGTTGATTTACTCTTCAAAACAAATCGAAAAGTAACCGTT
AAGCAATTAAAAGAAGATTATTTCAAAAAAATAGAATGTTTTGATAGTGTT
GAAATTTCAGGAGTTGAAGATAGATTTAATGCTTCATTAGGTACCTACCAT
GATTTGCTAAAAATTATTAAAGATAAAGATTTTTTGGATAATGAAGAAAAT
GAAGATATCTTAGAGGATATTGTTTTAACATTGACCTTATTTGAAGATAGG
GAGATGATTGAGGAAAGACTTAAAACATATGCTCACCTCTTTGATGATAAG
GTGATGAAACAGCTTAAACGTCGCCGTTATACTGGTTGGGGACGTTTGTCT
CGAAAATTGATTAATGGTATTAGGGATAAGCAATCTGGCAAAACAATATTA
GATTTTTTGAAATCAGATGGTTTTGCCAATCGCAATTTTATGCAGCTGATC
CATGATGATAGTTTGACATTTAAAGAAGACATTCAAAAAGCACAAGTGTCT
GGACAAGGCGATAGTTTACATGAACATATTGCAAATTTAGCTGGTAGCCCT
GCTATTAAAAAAGGTATTTTACAGACTGTAAAAGTTGTTGATGAATTGGTC
AAAGTAATGGGCGGCATAAGCCAGAAAATATCGTTATTGAAATGGCACGTG
AAAATCAGACAACTCAAAAGGGCCAGAAAAATTCGCGAGAGCGTATGAAAC
GAATCGAAGAAGGTATCAAAGAATTAGGAAGTCAGATTCTTAAAGAGCATC
CTGTTGAAAATACTCAATTGCAAAATGAAAAGCTCTATCTCTATTATCTCC
AAAATGGAAGAGACATGTATGTGGACCAAGAATTAGATATTAATCGTTTAA
GTGATTATGATGTCGATCACATTGTTCCACAAAGTTTCCTTAAAGACGATT
CAATAGACAATAAGGTCTTAACGCGTTCTGATAAAAATCGTGGTAAATCGG
ATAACGTTCCAAGTGAAGAAGTAGTCAAAAAGATGAAAAACTATTGGAGAC
AACTTCTAAACGCCAAGTTAATCACTCAACGTAAGTTTGATAATTTAACGA
AAGCTGAACGTGGAGGTTTGAGTGAACTTGATAAAGCTGGTTTTATCAAAC
GCCAATTGGTTGAAACTCGCCAAATCACTAAGCATGTGGCACAAATTTTGG
ATAGTCGCATGAATACTAAATACGATGAAAATGATAAACTTATTCGAGAGG
TTAAAGTGATTACCTTAAAATCTAAATTAGTTTCTGACTTCCGAAAAGATT
TCCAATTCTATAAAGTACGTGAGATTAACAATTACCATCATGCCCATGATG
CGTATCTAAATGCCGTCGTTGGAACTGCTTTGATTAAGAAATATCCAAAAC
TTGAATCGGAGTTTGTCTATGGTGATTATAAAGTTTATGATGTTCGTAAAA
TGATTGCTAAGTCTGAGCAAGAAATAGGCAAAGCAACCGCAAAATATTTCT
TTTACTCTAATATCATGAACTTCTTCAAAACAGAAATTACACTTGCAAATG
GAGAGATTCGCAAACGCCCTCTAATCGAAACTAATGGGGAAACTGGAGAAA
TTGTCTGGGATAAAGGGCGAGATTTTGCCACAGTGCGCAAAGTATTGTCCA
TGCCCCAAGTCAATATTGTCAAGAAAACAGAAGTACAGACAGGCGGATTCT
CCAAGGAGTCAATTTTACCAAAAAGAAATTCGGACAAGCTTATTGCTCGTA
AAAAAGACTGGGATCCAAAAAAATATGGTGGTTTTGATAGTCCAACGGTAG
CTTATTCAGTCTAGTGGTTGCTAAGGTGGAAAAAGGGAAATCGAAGAAGTT
AAAATCCGTTAAAGAGTTACTAGGGATCACAATTATGGAAAGAAGTTCCTT
TGAAAAAAATCCGATTGACTTTTTAGAAGCTAAAGGATATAAGGAAGTTAA
AAAAGACTTAATCATTAAACTACCTAAATATAGTCTTTTTGAGTTAGAAAA
CGGTCGTAAACGGATGCTGGCTAGTGCCGGAGAATTACAAAAAGGAAATGA
GCTGGCTCTGCCAAGCAAATATGTGAATTTTTTATATTTAGCTAGTCATTA
TGAAAAGTTGAAGGGTAGTCCAGAAGATAACGAACAAAAACAATTGTTTGT
GGAGCAGCATAAGCATTATTTAGATGAGATTATTGAGCAAATCAGTGAATT
TTCTAAGCGTGTTATTTTAGCAGATGCCAATTTAGATAAAGTTCTTAGTGC
ATATAACAAACATAGAGACAAACCAATACGTGAACAAGCAGAAAATATTAT
TCATTTATTTACGTTGACGAATCTTGGAGCTCCCGCTGCTTTTAAATATTT
TGATACAACAATTGATCGTAAACGATATACGTCTACAAAAGAAGTTTTAGA
TGCCACTCTTATCCATCAATCCATCACTGGTCTTTATGAAACACGCATTGA
TTTGAGTCAGCTAGGAGGTGACTGA
S. carneus ligD
SEQ ID NO: 71
ATCGAGGTCCGGCTGAGCAACCTGGACAAGGTGCTCTATCCGGCGACCGG
CACCACCAAGGGCGAGGTCATCGAGTACTACGCCGAAATCGCCCCGGCGA
TGCTGCCGCATATCGCGGGCCGGCCGATCACCCGGAAACGGTGGCCGAAC
GGTGTCGCCGAATCGTCGTTCTTCGAGAAGAACCTCGGCGCGGGTACACC
GTCGTGGCTACCGCGCCGTGCCCAGGAACATTCCGACCGCACCGCGCACT
ATCCGGTGATCTCGTCGCAGGCCGGCCTGGTCTGGCTGGGTCAGCAGGCC
GCCCTGGAGATCCACGTACCGAATGGCGCTTCGACGGCGATGCGCGCGGA
CCCGCGACGCGGCTGGTGTTCGATCTCGATCCCGGCCCCGGCGCGGGACT
GCCCGAATGCGCGCGGGTGGCGCTCGGGGTGCGGGATATGGTCGCCGAAA
TCGGGATGCGCGCGTTCCCGCTGACCAGCGGTAGCAAAGGTATCCACCTG
TACGTCCCGCTGGACCGGGTGCTGAGCCCCGGCGGGGCGTCCACGGTGGC
CAAACAGGTCGCCGCGAATCTGGAGAAACTCCTTCCCGACCTGGTCACCG
CCACCATCGCGAAGAGTGTGCGGGCCGGGAAGGTGTTCCTGGACTGGAGT
CAGAACAACCCGTCCAAGACGACCATCGCACCGTATTCGCTGCGCGGCCG
CGAGCAGCCGAACGTCGCCGCACCACGCCACTGGGCGGAGCTCGAGGACG
CCCGTGAACTGCGGCAGCTGCGGTTCGACGAAGTTCTGGAGCGTTATCGG
TCCGAGGGTGATCTGCTGGCCGGCCTGGATACACCCCTGAACGACGCGTT
GACGAAATACCGATCGATGCGTGACCCGGCGCGTACACCGGAGCCGGTAC
CGCCGCATTCGCCCCGGCCCGGCCCCGGTGACCGCTATGTCGTCCACGAA
CACCACGCCCGGCGGTTGCACTGGGATGTGCGGTTGGAACGCGACGGGGT
GCTGGTGTCGTGGGCGGTGCCCAAGGGGCCGCCGGAAAGCACCCGGCAGA
ATCGGCTCGCCGTGCACACCGAGGACCACCCGCTGGAATACCTGGACTTC
CACGGCACGATCCCGGCCGGCGAGTACGGGGCAGGGGAGCTGTCGGTCTG
GGATACCGGCACCTACCGCGCCGAGAAATGGCGCGACGACGAGGTGATCG
TGGTTTTCCGGGGCGAGCGGCTCAACGGCGGTACGCCATGATCCGGACCG
AGGGCGATCAATGGCTGATGCATCTCATGAAGGACCAGCCCGCGACCGGG
GAACTGCCGCGTGGACTCACCCCCATGCTGGCCACCAGTGGCGAAGTGGC
CGGGCTGCCGGACTCGGAGTGGGCGTTCGAACGTAAAGGGACGGATACCG
GCTGCTCGTCGAAATCGATGCCGGCGAAATGCGGCTGCGCAGCCGGGCCG
GTAACGACGTCACCGCGCGCTATCCCCAGTTGTCGGTGCTGGCCGAGGAG
CTGGCCGACCATCAGGTGATACTCGACGGTGAGCTCATCGTCCGCGGCCC
CGACGGCGCGGTGAATATCGCGCTGTTGAAGGCGAATCCGCGGCGCGCCG
AATTCCTGGCGTTCGATCTGCTGTTCCTCGACGGCACTTCACTGCTGCGC
AAACGCTACCGCGATCGGCGGCACGTGCTCGAAGCGCTGGCCGCGACCAC
CACCGAACTCCGGGTGCCACCGCGCTATGAGGGCGACGGCACCGAGGCCC
TGCACCGCAGCGAAGAAGATGGCGCCGAGGGCGTGATCGCCAAACGGCTG
GATTCGGTGTATCTGCCCGGGACCCGCGGGCATTCGTGGGTGAAGCACCG
GAACTGGCGTACCCAGGAGGTGGTGATCGGGGGTATGCGGCGCAGTAAGG
CGCGACCGTTCGCCTCGTTGCTGGTCGGGATACCGGCCGAGGACGGCCTG
GTGTATGCGGGCCGGGTCGGGACCGGGTTCGACGAAGCGGGGATGACCGA
ACTCGCGGCCCGGCTGCGCCGGTCGGAACGTAAGACGCCGCCGTTCACCA
ACGAGATGTCGGCCGATGAACTCCGGGACGCGATCTGGGTGACACCGAAG
ATCAAAGGCACTGTTCGCTACATGGATTGGACCGACGGCGGACGCTTCTG
GCATCCTGCCTGGCTCGGCGAGGTGTGA

### REFERENCES

Bentley S D, Chater K F, Cerdeno-Tarraga A M, Challis G L, Thomson N R, James K D, Harris D E, Quail M A, Kieser H, Harper D, Bateman A, Brown S, Chandra G, Chen C W, Collins M, Cronin A, Fraser A, Goble A, Hidalgo J, Hornsby T, Howarth S, Huang C H, Kieser T, Larke L, Murphy L, Oliver K, O'Neil S, Rabbinowitsch E, Rajandream M A, Rutherford K, Rutter S, Seeger K, Saunders D, Sharp S, Squares R, Squares S, Taylor K, Warren T, Wietzorrek A, Woodward J, Barrell B G, Parkhill J, Hopwood D A. 2002. Complete genome sequence of the model actinomycete Streptomyces coelicolor A3(2). Nature 417:141-147.

Cobb R E, Wang Y, Zhao H. 2014. High-Efficiency Multiplex Genome Editing of Streptomyces Species Using an Engineered CRISPR/Cas System. ACS synthetic biology.

Bikard, D., Euler, C. W., Jiang, W. Y., Nussenzweig, P. M., Goldberg, G. W., Duportet, X., Fischetti, V. A., and Marraffini, L. A. 2014. Exploiting CRISPR-Cas nucleases to produce sequence-specific antimicrobials. Nat.Biotechnol. 32:1146-1150.

Citorik, R. J., Mimee, M., and Lu, T. K. 2014. Sequence-specific antimicrobials using efficiently delivered RNA-guided nucleases. Nat. Biotechnol. 32:1141-1145.

Gomaa, A. A., Klumpe, H. E., Luo, M. L., Selle, K., Barrangou, R., and Beisel, C. L. 2014. Programmable removal of bacterial strains by use of genome targeting CRISPR-Cas systems. Mbio 5, e00928-13. DOI: 10.1128/mBio.00928-13.

Hilker R, Stadermann K B, Doppmeier D, Kalinowski J, Stoye J, Straube J, Winnebald J, Goesmann A. 2014. ReadXplorer—visualization and analysis of mapped sequences. Bioinformatics 30:2247-2254.

Huang H, Zheng G, Jiang W, Hu H, Lu Y. 2015. One-step high-efficiency CRISPR/Cas9-mediated genome editing in Streptomyces. Acta Biochim Biophys Sin (Shanghai).

Li H, Durbin R. 2009. Fast and accurate short read alignment with Burrows-Wheeler transform. Bioinformatics 25:1754-1760.

MacNeil D J, Occi J L, Gewain K M, MacNeil T, Gibbons P H, Ruby C L, Danis S J. 1992. Complex organization of the Streptomyces avermitilis genes encoding the avermectin polyketide synthase. Gene 115:119-125.

Muth G, Nussbaumer B, Wohlleben W, Puhler A. 1989. A Vector System with Temperature-Sensitive Replication for Gene Disruption and Mutational Cloning in Streptomycetes. Molecular & General Genetics 219:341-348.

Qi, L. S., Larson, M. H., Gilbert, L. A., Doudna, J. A., Weissman, J. S., Arkin, A. P., and Lim, W. A. 2013. Repurposing CRISPR as an RNA-guided platform for sequence-specific control of gene expression. Cell 152:1173-1183.

Rutherford K, Parkhill J, Crook J, Horsnell T, Rice P, Rajandream M A, Barrell B. 2000. Artemis: sequence visualization and annotation. Bioinformatics 16:944-945.

Items
1. A method for generating at least one deletion around at least one target nucleic acid sequence comprised within a host cell having a non-homologous end-joining (NHEJ) pathway which is at least partly deficient,
   said method comprising the step of inducing a CRISPR-Cas9 system in a host cell, wherein said CRISPR-Cas9 system is able to generate at least one break in said at least one target nucleic acid sequence and wherein the CRISPR-Cas9 system comprises a Cas9 nuclease and at least one guiding means,
   thereby generating at least one deletion around said at least one target nucleic acid sequence,
   wherein said at least one deletion is a deletion of at least 1 bp.
2. The method of item 1, further comprising the step of determining the size of the deletion.
3. The method of any one of the preceding items, wherein said at least one deletion is one deletion.
4. The method of any one of the preceding items, wherein said at least one target nucleic acid sequence is one target nucleic acid sequence.
5. The method of any one of the preceding items, wherein the guiding means comprises at least one sgRNA and/or at least one crRNA/tracrRNA set.
6. The method of any one of the preceding items, wherein the host cell is an archae, a prokaryotic cell or a eukaryotic cell.
7. The method of any one of the preceding items, wherein the NHEJ pathway of said host cell comprises at least one of four activities defined as:
   a DNA-binding activity,
   a primase activity,
   a ligase activity.
   a polymerase activity.
8. The method of item 7, wherein at least one is two or three.
9. The method of any one of items 7 or 8, wherein said host cell is naturally lacking at least one said four activities or wherein at least one of said four activities has been inactivated.
10. The method of any one of the preceding items, wherein the host cell is selected from the group consisting of actinobacteria.
11. The method of any one of the preceding items, wherein the host cell is selected from the group consisting of Actinomycetales, such as Streptomyces sp., Amycolatopsis sp. or Saccharopolyspora sp.
12. The method of any one of the preceding items, wherein the host cell is selected from the group consisting of Streptomyces coelicolor, Streptomyces avermitilis, Streptomyces aureofaciens, Streptomyces griseus, Streptomyces parvulus, Streptomyces albus, Streptomyces vinaceus, Streptomyces acrimycinis, Streptomyces calvuligerus, Streptomyces lividans, Streptomyces limosus, Streptomyces rubiqinosis, Streptomyces azureus, Streptomyces glaucenscens, Streptomyces rimosus, Streptomyces violaceoruber, Streptomyces kanamyceticus, Amycolatopsis orientalis, Amycolatopsis mediterranei and Saccharopolyspora erythraea.
13. The method of any one of the preceding items, wherein the at least one target nucleic acid sequence is comprised within a secondary metabolite biosynthetic gene.
14. The method of any one of the preceding items, wherein the at least one target nucleic acid sequence is comprised within a gene cluster such as a secondary metabolite gene cluster.
15. The method of any one of items 13 to 14, wherein the secondary metabolite is selected from the group consisting of antibiotics, herbicides, anti-cancer agents, immunosuppressants, flavors, parasiticides, enzymes and proteins.
16. The method of any one of items 13 to 15, wherein the secondary metabolite is an antibiotic selected from the group consisting of apramycin, bacitracin, chloramphenicol cephalosporins, cycloserine, erythromycin, fosfomycin, gentamicin, kanamycin, kirromycin, lassomycin, lincomycin, lysolipin, microbisporicin, neomycin, noviobiocin, nystatin, nitrofurantoin, platensimycin, pristinamycins, rifamycin, streptomycin, teicoplanin, tetracycline, tinidazole, ribostamycin, daptomycin, vancomycin, viomycin and virginiamycin.
17. The method of any one of items 13 to 15, wherein the secondary metabolite is a herbicide selected from the group consisting of bialaphos, resormycin and phosphinothricin.
18. The method of any one of items 13 to 15, wherein the secondary metabolite is an anti-cancer agent selected from the group consisting of doxorubicin, salinosporamides, aclarubicin, pentostatin, peplomycin, thrazarine and neocarcinostatin.
19. The method of any one of items 13 to 15, wherein the secondary metabolite is an immunosuppressant selected from the group consisting of rapamycin, FK520, FK506, cyclosporine, ushikulides, pentalenolactone I and hygromycin A.
20. The method of any one of items 13 to 15, wherein the secondary metabolite is a flavor such as geosmin.
21. The method of any one of items 13 to 15, wherein the secondary metabolite is a parasiticide such as an insecticide, an anthelmintic, a larvacide, or an antiprotozoal agent such as spinsad or avermectin.
22. The method of any one of items 1 to 12, wherein the at least one nucleic acid encodes an enzyme such as a metabolic enzyme selected from the group consisting of an amylase, a protease, a cellulase, a chitinase, a keratinase and a xylanase, a glycosyltransferase, an oxygenase, a hydroxylase, a methyltransferase, a dehydrogenase, a dehydratase.
23. The method of any one of the preceding items, wherein the generation of at least one deletion results in the inactivation of at least one gene.
24. The method of any one of the preceding items, wherein said deletion is a deletion of 1 to 1 500 000 bp, such as 1 to 1200000 bp, such as 1 to 1000000 bp, such as 1 to 500000 bp, such as 1 to 400000 bp, such as 1 to 300000 bp, such as 1 to 200000 bp, such as 1 to 100000 bp, such as 2 to 75000 bp, such as 3 to 50000 bp, such as 4 to 40000 bp, such as 5 to 30000 bp, such as 10 to 20000 bp, such as 25 to 10000 bp, such as 50 to 9000 bp, such as 75 to 8000 bp, such as 100 to 7000 bp, such as 150 to 6000 bp, such as 200 to 5000 bp, such as 250 to 4000 bp, such as 300 to 3000 bp, such as 400 to 2000 bp, such as 500 to 1000 bp, such as 600 to 900 bp, such as 700 to 800 bp.
25. The method of any one of the preceding items, wherein said deletion is a deletion of at least 1 bp, such as at least 2 bp, such as at least 3 bp, such as at least 4 bp, such as at least 5 bp, such as at least 10 bp, such as at least 15 bp, such as at least 20 bp, such as at least 50 bp, such as at least 100 bp, such as at least 250 bp, such as at least 500 bp.
26. The method of any one of the preceding items, wherein said deletion is a deletion of 1 to 100 bp, such as 1 to 75 bp, such as 1 to 50 bp, such as 1 to 40 bp, such as 1 to 30 bp, such as 1 to 20 bp, such as 1 to 10 bp, such as 1 to 9 bp, such as 1 to 8 bp, such as 1 to 7 bp, such as 1 to 6 bp, such as 1 to 5 bp, such as 1 to 4 bp, such as 1 to 3 bp, such as 1 to 2 bp.
27. A method for generating at least one indel around at least one target nucleic acid sequence comprised within a host cell having a non-homologous end-joining (NHEJ) pathway which is at least partly deficient, said method comprising the steps of:
i. restoring the full functionality of the NHEJ pathway in said host cell;
ii. inducing a CRISPR-Cas9 system in said host cell, wherein said CRISPR-Cas9 system is able to generate at least one break in said at least one target nucleic acid sequence and wherein the CRISPR-Cas9 system comprises a Cas9 nuclease and at least one guiding means,
   thereby generating at least one indel around said at least one target nucleic acid sequence,
   wherein said at least one indel is a deletion or insertion of at least 1 bp.
28. The method of item 27, further comprising the step of determining the size of the indel.
29. The method of any one of items 27 to 28, wherein said at least one indel is one indel.
30. The method of any one of items 27 to 29, wherein said at least one target nucleic acid sequence is one target nucleic acid sequence.
31. The method of item 30, wherein the guiding means is a single guide RNA (sgRNA).
32. The method of any one of items 27 to 31, wherein the host cell is an archaea, a prokaryotic cell or a eukaryotic cell.
33. The method of any one of items 27 to 32, wherein the NHEJ pathway of said host cell comprises at least one of four activities defined as:
   a DNA-binding activity,
   a primase activity,
   a ligase activity
   a polymerase activity.
34. The method of any one of items 27 to 33, wherein the NHEJ pathway of said host cell lacks the ligase activity.
35. The method of item 34, wherein the ligase activity is restored by expression of a functional ligase such as a heterologous ligase.
36. The method of item 35, wherein the heterologous ligase is derived from an organism selected from the group consisting of: Streptomyces carneus, Mycobacter tuberculosis, Nocardia spp., Smaragdicoccus niigatensis, Rhodococcus spp., Mycobacterium abscessus, Mycobacterium mageritense and Mycobacterium farcinogenes.
37. The method of any one of items 27 to 36, wherein the host cell is selected from the group consisting of actinobacteria.
38. The method of any one of items 27 to 37, wherein the host cell is selected from the group consisting of Actinomycetales, such as Streptomyces sp., Amycolatopsis sp. or Saccharopolyspora sp.
39. The method of any one of items 27 to 38, wherein the host cell is selected from the group consisting of Streptomyces coelicolor, Streptomyces avermitilis, Streptomyces aureofaciens, Streptomyces griseus, Streptomyces parvulus, Streptomyces albus, Streptomyces vinaceus, Streptomyces acrimycinis, Streptomyces calvuligerus, Streptomyces lividans, Streptomyces limosus, Streptomyces rubiqinosis, Streptomyces azureus, Streptomyces glaucenscens, Streptomyces rimosus, Streptomyces violaceoruber, Streptomyces kanamyceticus, Amycolatopsis orientalis, Amycolatopsis mediterranei and Saccharopolyspora erythraea.
40. The method of any one of items 27 to 39, wherein the at least one target nucleic acid sequence is comprised within a secondary metabolite biosynthetic gene.
41. The method of any one of items 27 to 40, wherein the at least one target nucleic acid sequence is comprised within a gene cluster such as a secondary metabolite gene cluster.
42. The method of any one of items 40 to 41, wherein the secondary metabolite is selected from the group consisting of antibiotics, herbicides, anti-cancer agents, immunosuppressants, flavors, parasiticides, enzymes and proteins.
43. The method of any one of items 40 to 42, wherein the secondary metabolite is an antibiotic selected from the group consisting of apramycin, bacitracin, chloramphenicol cephalosporins, cycloserine, erythromycin, fosfomycin, gentamicin, kanamycin, kirromycin, lassomycin, lincomycin, lysolipin, microbisporicin, neomycin, noviobiocin, nystatin, nitrofurantoin, platensimycin, pristinamycins, rifamycin, streptomycin, teicoplanin, tetracycline, tinidazole, ribostamycin, daptomycin, vancomycin, viomycin, virginiamycin.
44. The method of any one of items 40 to 42, wherein the secondary metabolite is a herbicide selected from the group consisting of bialaphos, resormycin and phosphinothricin.
45. The method of any one of items 40 to 42, wherein the secondary metabolite is an anti-cancer agent selected from the group consisting of doxorubicin, salinosporamides, aclarubicin, pentostatin, peplomycin, thrazarine and neocarcinostatin.
46. The method of any one of items 40 to 42, wherein the secondary metabolite is an immunosuppressant selected from the group consisting of rapamycin, FK520, FK506, cyclosporine, ushikulides, pentalenolactone I and hygromycin A.
47. The method of any one of items 40 to 42, wherein the secondary metabolite is a flavor such as geosmin.
48. The method of any one of items 40 to 42, wherein the secondary metabolite is a parasiticide such as an insecticide, an anthelmintic, a larvacide, or an antiprotozoal agent such as spinsad or avermectin.
49. The method of any one of items 27 to 39, wherein the at least one nucleic acid encodes an enzyme such as a metabolic enzyme selected from the group consisting of an amylase, a protease, a cellulase, a chitinase, a keratinase and a xylanase, a glycosyltransferase, an oxygenase, a hydroxylase, a methyltransferase, a dehydrogenase, a dehydratase.
50. The method of any one of items 27 to 49, wherein the generation of at least one indel results in the inactivation of at least one gene.
51. A method for selectively modulating transcription of at least one target nucleic acid sequence in a host cell, the method comprising introducing into the host cell:
i. at least one guiding means, or a nucleic acid comprising a nucleotide sequence encoding guiding means, wherein the guiding means comprises a nucleotide sequence that is complementary to a target nucleic acid sequence in the host cell; and
ii. a variant Cas9, or a nucleic acid comprising a nucleotide sequence encoding the variant Cas9, wherein the variant Cas9 has reduced endodeoxyribonuclease activity,
wherein said guiding means and said variant Cas9 form a complex in the host cell, said complex selectively modulating transcription of at least one target nucleic acid in the host cell.
52. The method of item 51, wherein the guiding means comprises at least one sgRNA and/or at least one crRNA/tracrRNA set.
53. The method of item 52, wherein the variant Cas9 can cleave one of the strands of the target nucleic acid sequence but has reduced ability to cleave the other strand of the target nucleic acid sequence.
54. The method of any one of items 51 to 53, wherein the variant Cas9 is selected from the group consisting of Cas9-H840A, Cas9-D10A and Cas9-H840A,D10A.
55. The method of any one of items 51 to 54, wherein the host cell is a prokaryotic cell selected from the group consisting of actinobacteria.
56. The method of any one of items 51 to 55, wherein the host cell is selected from the group consisting of Actinomycetales, such as Streptomyces sp., Amycolatopsis sp. or Saccharopolyspora sp.
57. The method of any one of items 51 to 56, wherein the host cell is selected from the group consisting of Streptomyces coelicolor, Streptomyces avermitilis, Streptomyces aureofaciens, Streptomyces griseus, Streptomyces parvulus, Streptomyces albus, Streptomyces vinaceus, Streptomyces acrimycinis, Streptomyces calvuligerus, Streptomyces lividans, Streptomyces limosus, Streptomyces rubiqinosis, Streptomyces azureus, Streptomyces glaucenscens, Streptomyces rimosus, Streptomyces violaceoruber, Streptomyces kanamyceticus, Amycolatopsis orientalis, Amycolatopsis mediterranei and Saccharopolyspora erythraea.
58. The method of any one items 51 to 57, wherein the at least one target nucleic acid sequence is comprised within a secondary metabolite biosynthetic gene.
59. The method of any one items 51 to 58, wherein the at least one target nucleic acid sequence is comprised within a gene cluster such as a secondary metabolite gene cluster.
60. The method of any one items 58 to 59, wherein the secondary metabolite is selected from the group consisting of antibiotics, herbicides, anti-cancer agents, immunosuppressants, flavors, parasiticides, enzymes and proteins.
61. The method of any one items 58 to 60, wherein the secondary metabolite is an antibiotic selected from the group consisting of apramycin, bacitracin, chloramphenicol cephalosporins, cycloserine, erythromycin, fosfomycin, gentamicin, kanamycin, kirromycin, lassomycin, lincomycin, lysolipin, microbisporicin, neomycin, noviobiocin, nystatin, nitrofurantoin, platensimycin, pristinamycins, rifamycin, streptomycin, teicoplanin, tetracycline, tinidazole, ribostamycin, daptomycin, vancomycin, viomycin, virginiamycin.
62. The method of any one items 58 to 60, wherein the secondary metabolite is a herbicide selected from the group consisting of bialaphos, resormycin and phosphinothricin.
63. The method of any one items 58 to 60, wherein the secondary metabolite is an anti-cancer agent selected from the group consisting of doxorubicin, salinosporamides, aclarubicin, pentostatin, peplomycin, thrazarine and neocarcinostatin.
64. The method of any one items 58 to 60, wherein the secondary metabolite is an immunosuppressant selected from the group consisting of rapamycin, FK520, FK506, cyclosporine, ushikulides, pentalenolactone I and hygromycin A.
65. The method of any one items 58 to 60, wherein the secondary metabolite is a flavor such as geosmin.
66. The method of any one items 58 to 60, wherein the secondary metabolite is a parasiticide such as an insecticide, an anthelmintic, a larvacide, or an antiprotozoal agent such as spinsad or avermectin.
67. The method of any one items 51 to 57, wherein the at least one nucleic acid encodes an enzyme such as a metabolic enzyme selected from the group consisting of an amylase, a protease, a cellulase, a chitinase, a keratinase and a xylanase, a glycosyltransferase, an oxygenase, a hydroxylase, a methyltransferase, a dehydrogenase, a dehydratase.
68. The method of any one of items 51 to 67, wherein:
i. the transcription of the guiding means is under the control of an inducible promoter; or
ii. the expression of the variant Cas9 is inducible.
69. A polynucleotide having at least 93% identity with SEQ ID NO: 1, such as at least 94% identity, such as at least 95% identity, such as at least 96% identity, such as at least 97% identity, such as at least 98% identity, such as at least 99% identity, such as 100% identity.
70. The polynucleotide of item 69, wherein the polynucleotide is non-naturally occurring.
71. A polypeptide encoded by the polynucleotide of any of items 69 to 70.
72. The polypeptide of any item 71, wherein the polypeptide is non-naturally occurring.
73. A cell comprising the polynucleotide of any of items 69 to 70.
74. A cell comprising the polypeptide of any of items 71 to 72.
75. A vector comprising the polynucleotide of any of items 69 to 70.
76. A clonal library obtainable by the method of any of items 1 to 26, said clonal library comprising a plurality of clones, each clone harbouring at least one deletion around at least one target nucleic acid sequence, wherein each of said deletion is a deletion of at least 1 bp.
77. A kit for performing the method of any of items 1 to 26, said kit comprising:
a vector comprising a nucleic acid sequence encoding a Cas9 nuclease or variant thereof; and
instructions for use.
78. The kit of item 77, wherein the nucleic acid sequence is the polynucleotide of items 69 to 70.
79. The kit of any one of items 77 to 78, further comprising at least one guiding means and/or at least one host cell.
80. The kit of any one of items 77 to 79, wherein the host cell has a non-homologous end-joining (NHEJ) pathway which is at least partly deficient.
81. The kit of any one of items 77 to 80, further comprising means for partly inactivating NHEJ in the host cell.
82. A kit for performing the method of any of items 27 to 50, said kit comprising:
a first vector comprising a nucleic acid sequence encoding Cas9 or a variant thereof; and
instructions for use.
83. The kit of item 82, further comprising a second vector comprising at least one nucleic acid encoding at least one of the NHEJ activities defined in item 33.
84. The kit of item 83, wherein the at least one nucleic acid encodes a ligase derived from S. carneus.
85. A kit for performing the method of any of items 51 to 68, said kit comprising:
   a vector comprising a nucleic acid sequence encoding a variant Cas9; and
   instructions for use.
86. The kit of item 85, wherein the variant Cas9 is Cas9-H840A, Cas9-D10A or Cas9-H840A,D10A.
87. The kit of any of items 85 to 86, further comprising at least one guiding means and/or at least one host cell.

1.-13. (canceled)

14. A method for generating at least one deletion around at least one target nucleic acid sequence comprised within a host cell having a non-homologous end-joining (NHEJ) pathway which is at least partly deficient, said method comprising the steps of:
(i) optionally, restoring the full functionality of the NHEJ pathway,
(ii) inducing a CRISPR-Cas9 system in said host cell, wherein said CRISPR-Cas9 system is able to generate at least one break in said at least one target nucleic acid sequence and wherein the CRISPR-Cas9 system comprises a Cas9 nuclease and at least one guiding means,
thereby generating:
a. if the method does not comprise step (i), at least one random-sized deletion around said at least one target nucleic acid sequence, wherein said at least one deletion is a random-sized deletion of at least 1 bp; or
b. if the method does comprise step (i), at least one indel around said at least one target nucleic acid sequence, wherein said at least one indel is a deletion or insertion of at least 1 bp.

15. The method of claim 14, wherein the host cell is an actinobacterium.

16. The method of claim 14, wherein the host cell is an Actinomycetales.

17. The method of claim 14, wherein the host cell is selected from the group consisting of: Streptomyces coelicolor, Streptomyces avermitilis, Streptomyces aureofaciens, Streptomyces griseus, Streptomyces parvulus, Streptomyces albus, Streptomyces vinaceus, Streptomyces acrimycinis, Streptomyces calvuligerus, Streptomyces lividans, Streptomyces limosus, Streptomyces rubiqinosis, Streptomyces azureus, Streptomyces glaucenscens, Streptomyces rimosus, Streptomyces violaceoruber, Streptomyces kanamyceticus, Amycolatopsis orientalis, Amycolatopsis mediterranei, and Saccharopolyspora erythraea.

18. The method of claim 14, wherein the NHEJ pathway of said host cell comprises at least one of four activities selected from the group consisting of: a DNA-binding activity, a primase activity, a ligase activity, and a polymerase activity.

19. The method of claim 18, wherein the NHEJ pathway of said host cell comprises at least two of the four activities or at least three of the four activities.

20. The method of claim 14, wherein the at least one target nucleic acid sequence is comprised within a secondary metabolite biosynthetic gene or within a secondary metabolite gene cluster.

21. The method of claim 20, wherein the secondary metabolite is selected from the group consisting of: antibiotics, herbicides, anti-cancer agents, immunosuppressants, flavors, parasiticides, enzymes, and proteins.

22. The method of claim 20, wherein the secondary metabolite is an antibiotic selected from the group consisting of: apramycin, bacitracin, chloramphenicol cephalosporins, cycloserine, erythromycin, fosfomycin, gentamicin, kanamycin, kirromycin, lassomycin, lincomycin, lysolipin, microbisporicin, neomycin, noviobiocin, nystatin, nitrofurantoin, platensimycin, pristinamycins, rifamycin, streptomycin, teicoplanin, tetracycline, tinidazole, ribostamycin, daptomycin, vancomycin, viomycin, and virginiamycin.

23. The method of claim 20, wherein the secondary metabolite is a herbicide selected from the group consisting of: bialaphos, resormycin, and phosphinothricin.

24. The method of claim 20, wherein the secondary metabolite is an anti-cancer agent selected from the group consisting of: doxorubicin, salinosporamides, aclarubicin, pentostatin, peplomycin, thrazarine, and neocarcinostatin.

25. The method of claim 20, wherein the secondary metabolite is an immunosuppressant selected from the group consisting of: rapamycin, FK520, FK506, cyclosporine, ushikulides, pentalenolactone I, and hygromycin A.

26. The method of claim 20, wherein the secondary metabolite is a flavor.

27. The method of claim 20, wherein the secondary metabolite is a parasiticide selected from the group consisting of: an insecticide, an anthelmintic, and a larvacide; or wherein the secondary metabolite is an antiprotozoal agent selected from the group consisting of: spinsad, and avermectin.

28. The method of claim 14, wherein the at least one target nucleic acid encodes an enzyme.

29. The method of claim 28, wherein the enzyme is selected from the group consisting of: an amylase, a protease, a cellulase, a chitinase, a keratinase and a xylanase, a glycosyltransferase, an oxygenase, a hydroxylase, a methyltransferase, a dehydrogenase, and a dehydratase.

30. A polypeptide encoded by a polynucleotide encoding a Cas9 nuclease or a variant thereof and having at least 94% identity with SEQ ID NO: 1.

31. The polypeptide of claim 30, wherein polynucleotide sequence is codon-optimized for Streptomycetes.

32. A method for selectively modulating transcription of at least one target nucleic acid sequence in a host cell, the method comprising introducing into the host cell:
(i) at least one guiding means, or a nucleic acid comprising a nucleotide sequence encoding guiding means, wherein the guiding means comprises a nucleotide sequence that is complementary to a target nucleic acid sequence in the host cell; and
(ii) a variant Cas9, or a nucleic acid comprising a nucleotide sequence encoding the variant Cas9, wherein the variant Cas9 is a variant of the polypeptide of claim 17, with reduced endodeoxyribonuclease activity and is codon-optimized for Streptomycetes,
wherein said guiding means and said variant Cas9 form a complex in the host cell, said complex selectively modulating transcription of at least one target nucleic acid in the host cell.

33. The method of claim 19, wherein the host cell is an actinobacterium.


SEQUENCE LISTING (72 sequences)

SEQ ID NO: 1 - DNA, 4107 bp, artificial sequence
  “Codon- optimised Cas9 sequence”
  Codon- optimised Cas9 sequence
  CDS 1..4107: /note=“Codon- optimised Cas9 sequence” /transl_table=1
atggacaagaagtactccatcggcctcgacatcggcaccaactccgtgggctgggcggtcatcaccgacgagtacaaggtcccctccaagaagttcaaggtcctgggcaacaccgaccggcactcgatcaagaagaacctgatcggcgccctgctcttcgacagcggcgagaccgccgaggcgacccgcctgaagcggaccgcgcgtcgccgctacacccggcgcaagaaccgcatctgctacctgcaggaaatcttctccaacgagatggccaaggtggacgactcgttcttccaccgcctggaggagagcttcctggtggaggaggacaagaagcacgagcgccacccgatcttcggcaacatcgtggacgaggtggcctaccacgagaagtaccccaccatctaccacctccgcaagaagctggtggactcgaccgacaaggcggacctgcggctcatctacctggccctcgcgcacatgatcaagttccgcggccacttcctcatcgagggcgacctgaacccggacaactccgacgtggacaagctcttcatccagctggtgcagacctacaaccagctgttcgaggagaaccccatcaacgccagcggcgtggacgccaaggcgatcctctccgcgcgcctgagcaagtcccggcgcctggagaacctcatcgcccagctgccgggcgagaagaagaacggcctcttcggcaacctgatcgcgctgtcgctcggcctgacccccaacttcaagagcaacttcgacctggccgaggacgcgaagctccagctgtccaaggacacctacgacgacgacctggacaacctgctcgcccagatcggcgaccagtacgcggacctcttcctggccgcgaagaacctctcggacgccatcctgctcagcgacatcctgcgggtcaacaccgagatcaccaaggccccgctgtcggcgagcatgatcaagcggtacgacgagcaccaccaggacctgaccctgctcaaggccctcgtgcgccagcagctgcccgagaagtacaaggaaatcttcttcgaccagtccaagaacggctacgccggctacatcgacggcggcgcgtcgcaggaggagttctacaagttcatcaagccgatcctggagaagatggacggcaccgaggagctgctcgtcaagctgaaccgcgaggacctgctccgcaagcagcggaccttcgacaacggctccatcccgcaccagatccacctgggcgagctccacgccatcctccggcgccaggaggacttctaccccttcctgaaggacaaccgcgagaagatcgagaagatcctgaccttccgcatcccgtactacgtcggccccctggcccgcggcaactcccggttcgcgtggatgacccggaagtcggaggagaccatcaccccgtggaacttcgaggaggtcgtggacaagggcgcgtccgcgcagtcgttcatcgagcgcatgaccaacttcgacaagaacctcccgaacgagaaggtcctgcccaagcactccctgctctacgagtacttcaccgtgtacaacgagctgaccaaggtcaagtacgtgaccgagggcatgcggaagccggccttcctgtcgggcgagcagaagaaggcgatcgtggacctgctcttcaagaccaaccgcaaggtcaccgtgaagcagctgaaggaggactacttcaagaagatcgagtgcttcgactccgtcgagatcagcggcgtggaggaccgcttcaacgcctccctgggcacctaccacgacctgctcaagatcatcaaggacaaggacttcctcgacaacgaggagaacgaggacatcctggaggacatcgtcctcaccctgaccctcttcgaggaccgcgagatgatcgaggagcggctcaagacctacgcccacctgttcgacgacaaggtgatgaagcagctgaagcgtcgccgctacaccggctggggccgcctctcccggaagctgatcaacggcatccgggacaagcagagcggcaagaccatcctggacttcctcaagtccgacggcttcgccaaccgcaacttcatgcagctcatccacgacgacagcctgaccttcaaggaggacatccagaaggcccaggtctcgggccagggcgacagcctccacgagcacatcgccaacctggcgggctccccggcgatcaagaagggcatcctccagaccgtcaaggtcgtggacgagctggtcaaggtgatgggccgccacaagcccgagaacatcgtgatcgagatggcccgggagaaccagaccacccagaagggccagaagaactcgcgcgagcggatgaagcggatcgaggagggcatcaaggagctcggcagccagatcctgaaggagcacccggtcgagaacacccagctgcagaacgagaagctgtacctctactacctgcagaacggccgcgacatgtacgtggaccaggagctcgacatcaaccggctgtccgactacgacgtggaccacatcgtgccgcagtccttcctgaaggacgactcgatcgacaacaaggtcctgacccgctcggacaagaaccggggcaagtccgacaacgtgccctcggaggaggtcgtgaagaagatgaagaactactggcgccagctgctcaacgccaagctcatcacccagcgcaagttcgacaacctgaccaaggccgagcggggcggcctgagcgagctcgacaaggcgggcttcatcaagcgccagctggtcgagacccggcagatcaccaagcacgtggcccagatcctggactcccggatgaacaccaagtacgacgagaacgacaagctgatccgcgaggtcaaggtgatcaccctcaagagcaagctggtctccgacttccgcaaggacttccagttctacaaggtccgggagatcaacaactaccaccacgcccacgacgcgtacctgaacgccgtcgtgggcaccgcgctgatcaagaagtacccgaagctggagtccgagttcgtctacggcgactacaaggtctacgacgtgcgcaagatgatcgccaagagcgagcaggagatcggcaaggccaccgcgaagtacttcttctactccaacatcatgaacttcttcaagaccgagatcaccctggccaacggcgagatccgcaagcggcccctgatcgagaccaacggcgagaccggcgagatcgtctgggacaagggccgcgacttcgccaccgtccggaaggtgctgtcgatgccgcaggtcaacatcgtgaagaagaccgaggtgcagaccggcggcttcagcaaggagtccatcctccccaagcgcaacagcgacaagctgatcgcccggaagaaggactgggacccgaagaagtacggcggcttcgacagccccaccgtcgcctactccgtgctggtcgtggcgaaggtcgagaagggcaagagcaagaagctgaagtccgtgaaggagctgctcggcatcaccatcatggagcgctcctcgttcgagaagaacccgatcgacttcctggaggccaagggctacaaggaggtcaagaaggacctcatcatcaagctgcccaagtacagcctgttcgagctggagaacggccgcaagcggatgctcgcctccgcgggcgagctgcagaagggcaacgagctggccctcccgtcgaagtacgtcaacttcctgtacctcgcgtcccactacgagaagctgaagggctcgcccgaggacaacgagcagaagcagctcttcgtggagcagcacaagcactacctggacgagatcatcgagcagatcagcgagttcagcaagcgcgtcatcctggccgacgcgaacctcgacaaggtgctgtccgcctacaacaagcaccgcgacaagccgatccgggagcaggcggagaacatcatccacctgttcaccctcaccaacctgggcgcccccgccgcgttcaagtacttcgacaccaccatcgaccgcaagcggtacacctccaccaaggaggtcctcgacgcgaccctgatccaccagagcatcaccggcctgtacgagacccgcatcgacctgtcccagctcggcggcgactga

SEQ ID NO: 2 - protein, 1368 aa, artificial sequence [sequence omitted]

SEQ ID NO: 3 - DNA, 4107 bp, Streptococcus pyogenes
  CDS 1..4107: /note=“Cas9 sequence” /transl_table=11
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 4 - DNA, 20 bp, artificial sequence
  “Target sequence”
  Target sequence
  misc_binding 1..20: /note=“ActIorf1-1 NT”
gtggctcgaaggaggctcga

SEQ ID NO: 5 - DNA, 20 bp, artificial sequence
  “Target sequence”
  Target sequence
  misc_binding 1..20: /note=“ActIorf1-2 T”
agctcgatcaagtcgatggt

SEQ ID NO: 6 - DNA, 20 bp, artificial sequence
  “Target sequence”
  Target sequence
  misc_binding 1..20: /note=“ActIorf1-3 T”
gaagcgcagagtcgtcatca

SEQ ID NO: 7 - DNA, 20 bp, artificial sequence
  “Target sequence”
  Target sequence
  misc_binding 1..20: /note=“ActIorf1-4 T”
cccctcgccctaccgttcac

SEQ ID NO: 8 - DNA, 20 bp, artificial sequence
  “Target sequence”
  Target sequence
  misc_binding 1..20: /note=“ActIorf1-5 T”
gcgcgagtatctgctgctgt

SEQ ID NO: 9 - DNA, 20 bp, artificial sequence
  “Target sequence”
  Target sequence
  misc_binding 1..20: /note=“ActIorf1-6 T”
ctgcaacgcgtaccacatga

SEQ ID NO: 10 - DNA, 20 bp, artificial sequence
  “Target sequence”
  Target sequence
  misc_binding 1..20: /note=“Actvb-1 NT”
tcgccgcaactgtcgaacac

SEQ ID NO: 11 - DNA, 20 bp, artificial sequence
  “Target sequence”
  Target sequence
  misc_binding 1..20: /note=“Actvb-2 NT”
ctgccatcttcgaactccct

SEQ ID NO: 12 - DNA, 20 bp, artificial sequence
  “Target sequence”
  Target sequence
  misc_binding 1..20: /note=“Actvb-3 T”
ttcccggtgttcgacagttg

SEQ ID NO: 13 - DNA, 20 bp, artificial sequence
  “Target sequence”
  Target sequence
  misc_binding 1..20: /note=“Actvb-4 T”
actggtctgcctggctcgta

SEQ ID NO: 14 - DNA, 20 bp, artificial sequence
  “Target sequence”
  Target sequence
  misc_binding 1..20: /note=“Actvb-5 NT”
atcttcgaactccctaggcg

SEQ ID NO: 15 - DNA, 20 bp, artificial sequence
  “Target sequence”
  Target sequence
  misc_binding 1..20: /note=“Actvb-6 NT”
gtcccggagcattccctggt

SEQ ID NO: 16 - DNA, 20 bp, artificial sequence
  “Target sequence”
  Target sequence
  misc_binding 1..20: /note=“orf1p-S1 T”
gtgttcccctccctgcctcg

SEQ ID NO: 17 - DNA, 20 bp, artificial sequence
  “Target sequence”
  Target sequence
  misc_binding 1..20: /note=“orf1p-S3 T”
tccctcacgcgctcagcttt

SEQ ID NO: 18 - DNA, 20 bp, artificial sequence
  “Target sequence”
  Target sequence
  misc_binding 1..20: /note=“orf1p-S5 T”
ctttgggcgcccggctcgag

SEQ ID NO: 19 - DNA, 20 bp, artificial sequence
  “Target sequence”
  Target sequence
  misc_binding 1..20: /note=“orf1p-A1 NT”
ccttcgaccgccgctcgagc

SEQ ID NO: 20 - DNA, 20 bp, artificial sequence
  “Target sequence”
  Target sequence
  misc_binding 1..20: /note=“orf1p-A4 NT”
gcccaaagctgagcgcgtga

SEQ ID NO: 21 - DNA, 20 bp, artificial sequence
  “Target sequence”
  Target sequence
  misc_binding 1..20: /note=“orf1p-A5 NT”
tgagcgcgtgagggaccacg

SEQ ID NO: 22 - DNA, 20 bp, artificial sequence
  “Target sequence”
  Target sequence
  misc_binding 1..20: /note=“ActIorf1-7 NT”
ttcgagcctccttcgagcca

SEQ ID NO: 23 - DNA, 20 bp, artificial sequence
  “Target sequence”
  Target sequence
  misc_binding 1..20: /note=“ActIorf1-8 NT”
ggcatcgaggggtcccgtat

SEQ ID NO: 24 - DNA, 50 bp, artificial sequence
  “Primer”
  Primer
  primer_bind 1..50: /note=“ActIorf1-F1”
catgccatgggtggctcgaaggaggctcgagttttagagctagaaatagc

SEQ ID NO: 25 - DNA, 50 bp, artificial sequence
  “Primer”
  Primer
  primer_bind 1..50: /note=“ActIorf1-F2”
catgccatggagctcgatcaagtcgatggtgttttagagctagaaatagc

SEQ ID NO: 26 - DNA, 50 bp, artificial sequence
  “Primer”
  Primer
  primer_bind 1..50: /note=“ActIorf1-F3”
catgccatgggaagcgcagagtcgtcatcagttttagagctagaaatagc

SEQ ID NO: 27 - DNA, 50 bp, artificial sequence
  “Primer”
  Primer
  primer_bind 1..50: /note=“ActIorf1-F4”
catgccatggcccctcgccctaccgttcacgttttagagctagaaatagc

SEQ ID NO: 28 - DNA, 50 bp, artificial sequence
  “Primer”
  Primer
  primer_bind 1..50: /note=“ActIorf1-F5”
catgccatgggcgcgagtatctgctgctgtgttttagagctagaaatagc

SEQ ID NO: 29 - DNA, 50 bp, artificial sequence
  “Primer”
  Primer
  primer_bind 1..50: /note=“ActIorf1-F6”
catgccatggctgcaacgcgtaccacatgagttttagagctagaaatagc

SEQ ID NO: 30 - DNA, 50 bp, artificial sequence
  “Primer”
  Primer
  primer_bind 1..50: /note=“ActIorf1-F7”
catgccatggttcgagcctccttcgagccagttttagagctagaaatagc

SEQ ID NO: 31 - DNA, 50 bp, artificial sequence
  “Primer”
  Primer
  primer_bind 1..50: /note=“ActIorf1-F8”
catgccatggggcatcgaggggtcccgtatgttttagagctagaaatagc

SEQ ID NO: 32 - DNA, 50 bp, artificial sequence
  “Primer”
  Primer
  primer_bind 1..50: /note=“ActVB-F1”
catgccatggtcgccgcaactgtcgaacacgttttagagctagaaatagc

SEQ ID NO: 33 - DNA, 50 bp, artificial sequence
  “Primer”
  Primer
  primer_bind 1..50: /note=“ActVB-F2”
catgccatggctgccatcttcgaactccctgttttagagctagaaatagc

SEQ ID NO: 34 - DNA, 50 bp, artificial sequence
  “Primer”
  Primer
  primer_bind 1..50: /note=“ActVB-F3”
catgccatggttcccggtgttcgacagttggttttagagctagaaatagc

SEQ ID NO: 35 - DNA, 50 bp, artificial sequence
  “Primer”
  Primer
  primer_bind 1..50: /note=“ActVB-F4”
catgccatggactggtctgcctggctcgtagttttagagctagaaatagc

SEQ ID NO: 36 - DNA, 50 bp, artificial sequence
  “Primer”
  Primer
  primer_bind 1..50: /note=“ActVB-F5”
catgccatggatcttcgaactccctaggcggttttagagctagaaatagc

SEQ ID NO: 37 - DNA, 50 bp, artificial sequence
  “Primer”
  Primer
  primer_bind 1..50: /note=“ActVB-F6”
catgccatgggtcccggagcattccctggtgttttagagctagaaatagc

SEQ ID NO: 38 - DNA, 50 bp, artificial sequence
  “Primer”
  Primer
  primer_bind 1..50: /note=“orf1p-S1 T-F”
catgccatgggtgttcccctccctgcctcggttttagagctagaaatagc

SEQ ID NO: 39 - DNA, 50 bp, artificial sequence
  “Primer”
  Primer
  primer_bind 1..50: /note=“orf1p-S3 T-F”
catgccatggtccctcacgcgctcagctttgttttagagctagaaatagc

SEQ ID NO: 40 - DNA, 50 bp, artificial sequence
  “Primer”
  Primer
  primer_bind 1..50: /note=“orf1p-S5 T-F”
catgccatggctttgggcgcccggctcgaggttttagagctagaaatagc

SEQ ID NO: 41 - DNA, 50 bp, artificial sequence
  “Primer”
  Primer
  primer_bind 1..50: /note=“orf1p-A1 NT-F”
catgccatggccttcgaccgccgctcgagcgttttagagctagaaatagc

SEQ ID NO: 42 - DNA, 50 bp, artificial sequence
  “Primer”
  Primer
  primer_bind 1..50: /note=“orf1p-A4 NT-F”
catgccatgggcccaaagctgagcgcgtgagttttagagctagaaatagc

SEQ ID NO: 43 - DNA, 50 bp, artificial sequence
  “Primer”
  Primer
  primer_bind 1..50: /note=“orf1p-A5 NT-F”
catgccatggtgagcgcgtgagggaccacggttttagagctagaaatagc

SEQ ID NO: 44 - DNA, 33 bp, artificial sequence
  “Primer”
  Primer
  primer_bind 1..33: /note=“sgRNA-R”
acgcctacgtaaaaaaagcaccgactcggtgcc

SEQ ID NO: 45 - DNA, 18 bp, artificial sequence
  “Primer”
  Primer
  primer_bind 1..18: /note=“gRNA check-F”
acatgtgcggtcgatctt

SEQ ID NO: 46 - DNA, 20 bp, artificial sequence
  “Primer”
  Primer
  primer_bind 1..20: /note=“gRNA check-R”
tacgtaaaaaaagcaccgac

SEQ ID NO: 47 - DNA, 39 bp, artificial sequence
  “Primer”
  Primer
  primer_bind 1..39: /note=“orf1-5′F”
tcgtcgaaggcactagaaggcatccgctgaacgagaccc

SEQ ID NO: 48 - DNA, 40 bp, artificial sequence
  “Primer”
  Primer
  primer_bind 1..40: /note=“orf1-5′R”
gctcacgtcgaagcgggtgaccacgcaggactccgaagtc

SEQ ID NO: 49 - DNA, 19 bp, artificial sequence
  “Primer”
  Primer
  primer_bind 1..19: /note=“orf1-3′F”
tcacccgcttcgacgtgag

SEQ ID NO: 50 - DNA, 38 bp, artificial sequence
  “Primer”
  Primer
  primer_bind 1..38: /note=“orf1-3′R”
ggtcgatccccgcatataggttcgccgagcaccaggtc

SEQ ID NO: 51 - DNA, 39 bp, artificial sequence
  “Primer”
  Primer
  primer_bind 1..39: /note=“VB-5′F”
tcgtcgaaggcactagaaggcgactcgctcgccctgatg

SEQ ID NO: 52 - DNA, 41 bp, artificial sequence
  “Primer”
  Primer
  primer_bind 1..41: /note=“VB-5′R”
caccaacctgctcgggctgcgccgtggaagtgggtgttgac

SEQ ID NO: 53 - DNA, 18 bp, artificial sequence
  “Primer”
  Primer
  primer_bind 1..18: /note=“VB-3′F”
gcagcccgagcaggttgg

SEQ ID NO: 54 - DNA, 38 bp, artificial sequence
  “Primer”
  Primer
  primer_bind 1..38: /note=“VB-3′R”
ggtcgatccccgcatataggtccgttgcggcgtccatc

SEQ ID NO: 55 - DNA, 19 bp, artificial sequence
  “Primer”
  Primer
  primer_bind 1..19: /note=“VB-check-F”
cggctggtgcgtcagcaac

SEQ ID NO: 56 - DNA, 18 bp, artificial sequence
  “Primer”
  Primer
  primer_bind 1..18: /note=“VB-check-R”
acgtggcgggtcgaacgg

SEQ ID NO: 57 - DNA, 20 bp, artificial sequence
  “Primer”
  Primer
  primer_bind 1..20: /note=“ORF1-check-F”
ccgccttgaggacctgtttg

SEQ ID NO: 58 - DNA, 19 bp, artificial sequence
  “Primer”
  Primer
  primer_bind 1..19: /note=“ORF1-check-R”
acacgctgaccgacttggg

SEQ ID NO: 59 - DNA, 20 bp, artificial sequence
  “Primer”
  Primer
  primer_bind 1..20: /note=“CAS9-check-F”
tccacgagcacatcgccaac

SEQ ID NO: 60 - DNA, 22 bp, artificial sequence
  “Primer”
  Primer
  primer_bind 1..22: /note=“CAS9-check-R”
gaccttgtagtcgccgtagacg

SEQ ID NO: 61 - DNA, 40 bp, artificial sequence
  “Primer”
  Primer
  primer_bind 1..40: /note=“ScaligD-F”
tcgtcgaaggcactagaagggcggtcgatcttgacggctg

SEQ ID NO: 62 - DNA, 40 bp, artificial sequence
  “Primer”
  Primer
  primer_bind 1..40: /note=“ScaligD-R”
ggtcgatccccgcatataggtgccgccgggcgttttttat

SEQ ID NO: 63 - DNA, 20 bp, artificial sequence
  “Primer”
  Primer
  primer_bind 1..20: /note=“orf1-6 ligD test-F”
ccgccgacaccccgatcacc

SEQ ID NO: 64 - DNA, 20 bp, artificial sequence
  “Primer”
  Primer
  primer_bind 1..20: /note=“orf1-6 ligD test-R”
accgcagcttccgctccctg

SEQ ID NO: 65 - DNA, 20 bp, artificial sequence
  “Primer”
  Primer
  primer_bind 1..20: /note=“vb2 ligD test-F”
cgaggtgatcgacgccaacc

SEQ ID NO: 66 - DNA, 20 bp, artificial sequence
  “Primer”
  Primer
  primer_bind 1..20: /note=“vb2 ligD test-R”
tcgccgagcaggatgatgtg

SEQ ID NO: 67 - DNA, 82 bp, artificial sequence
  “core guide RNA sequence”
  core guide RNA sequence
  misc_binding 1..82: /note=“core guide RNA”
gttttagagctagaaatagcaagttaaaataaggctagtccgttatcaacttgaaaaagtggcaccgagtcggtgctttttt

SEQ ID NO: 68 - DNA, 228 bp, Artificial sequence
  “sgRNA scaffold”
  sgRNA scaffold
  misc_feature 1..228: /note=“sgRNA scaffold; n is A, T, G or C”
gcggtcgatcttgacggctggcgagaggtgcggggaggatctgaccgacgcggtccacacgtggcaccgcgatgctgttgtgggcacaatcgtgccggttggtaggatcgacggccatggnnnnnnnnnnnnnnnnnnnngttttagagctagaaatagcaagttaaaataaggctagtccgttatcaacttgaaaaagtggcaccgagtcggtgctttttttacgta

SEQ ID NO: 69 - DNA, 50 bp, Artificial sequence
  “Primer”
  Primer
  misc_binding 1..50: /note=“Target-specific Fw primer; n is A, T, G or C”
  misc_feature 11..30: /note=“n is a, c, g, or t”
catgccatggnnnnnnnnnnnnnnnnnnnngttttagagctagaaatagc

SEQ ID NO: 70 - protein, 1368 aa, Streptococcus pyogenes [sequence omitted]

SEQ ID NO: 71 - DNA, 2181 bp, Streptomyces carneus
  “ligD”
  ligD
  CDS 1..2181: /transl_table=1
atcgaggtccggctgagcaacctggacaaggtgctctatccggcgaccggcaccaccaagggcgaggtcatcgagtactacgccgaaatcgccccggcgatgctgccgcatatcgcgggccggccgatcacccggaaacggtggccgaacggtgtcgccgaatcgtcgttcttcgagaagaacctcggcgcgggtacaccgtcgtggctaccgcgccgtgcccaggaacattccgaccgcaccgcgcactatccggtgatctcgtcgcaggccggcctggtctggctgggtcagcaggccgccctggagatccacgtaccgcaatggcgcttcgacggcgatgcgcgcggacccgcgacgcggctggtgttcgatctcgatcccggccccggcgcgggactgcccgaatgcgcgcgggtggcgctcggggtgcgggatatggtcgccgaaatcgggatgcgcgcgttcccgctgaccagcggtagcaaaggtatccacctgtacgtcccgctggaccgggtgctgagccccggcggggcgtccacggtggccaaacaggtcgccgcgaatctggagaaactccttcccgacctggtcaccgccaccatcgcgaagagtgtgcgggccgggaaggtgttcctggactggagtcagaacaacccgtccaagacgaccatcgcaccgtattcgctgcgcggccgcgagcagccgaacgtcgccgcaccacgccactgggcggagctcgaggacgcccgtgaactgcggcagctgcggttcgacgaagttctggagcgttatcggtccgagggtgatctgctggccggcctggatacacccctgaacgacgcgttgacgaaataccgatcgatgcgtgacccggcgcgtacaccggagccggtaccgccgcattcgccccggcccggccccggtgaccgctatgtcgtccacgaacaccacgcccggcggttgcactgggatgtgcggttggaacgcgacggggtgctggtgtcgtgggcggtgcccaaggggccgccggaaagcacccggcagaatcggctcgccgtgcacaccgaggaccacccgctggaatacctggacttccacggcacgatcccggccggcgagtacggggcaggggagctgtcggtctgggataccggcacctaccgcgccgagaaatggcgcgacgacgaggtgatcgtggttttccggggcgagcggctcaacggccggtacgccatgatccggaccgagggcgatcaatggctgatgcatctcatgaaggaccagcccgcgaccggggaactgccgcgtggactcacccccatgctggccaccagtggcgaagtggccgggctgccggactcggagtgggcgttcgaacgtaaatgggacggataccggctgctcgtcgaaatcgatgccggcgaaatgcggctgcgcagccgggccggtaacgacgtcaccgcgcgctatccccagttgtcggtgctggccgaggagctggccgaccatcaggtgatactcgacggtgagctcatcgtccgcggccccgacggcgcggtgaatatcgcgctgttgaaggcgaatccgcggcgcgccgaattcctggcgttcgatctgctgttcctcgacggcacttcactgctgcgcaaacgctaccgcgatcggcggcacgtgctcgaagcgctggccgcgaccaccaccgaactccgggtgccaccgcgctatgagggcgacggcaccgaggccctgcaccgcagcgaagaagatggcgccgagggcgtgatcgccaaacggctggattcggtgtatctgcccgggacccgcgggcattcgtgggtgaagcaccggaactggcgtacccaggaggtggtgatcgggggtatgcggcgcagtaaggcgcgaccgttcgcctcgttgctggtcgggataccggccgaggacggcctggtgtatgcgggccgggtcgggaccgggttcgacgaagcggggatgaccgaactcgcggcccggctgcgccggtcggaacgtaagacgccgccgttcaccaacgagatgtcggccgatgaactccgggacgcgatctgggtgacaccgaagatcaaaggcactgttcgctacatggattggaccgacggcggacgcttctggcatcctgcctggctcggcgaggtgtga

SEQ ID NO: 72 - protein, 726 aa, Streptomyces carneus
  “[CDS]1..2181 from SEQ ID NO 71”
  [CDS]1..2181 from SEQ ID NO 71
IEVRLSNLDKVLYPATGTTKGEVIEYYAEIAPAMLPHIAGRPITRKRWPNGVAESSFFEKNLGAGTPSWLPRRAQEHSDRTAHYPVISSQAGLVWLGQQAALEIHVPQWRFDGDARGPATRLVFDLDPGPGAGLPECARVALGVRDMVAEIGMRAFPLTSGSKGIHLYVPLDRVLSPGGASTVAKQVAANLEKLLPDLVTATIAKSVRAGKVFLDWSQNNPSKTTIAPYSLRGREQPNVAAPRHWAELEDARELRQLRFDEVLERYRSEGDLLAGLDTPLNDALTKYRSMRDPARTPEPVPPHSPRPGPGDRYVVHEHHARRLHWDVRLERDGVLVSWAVPKGPPESTRQNRLAVHTEDHPLEYLDFHGTIPAGEYGAGELSVWDTGTYRAEKWRDDEVIVVFRGERLNGRYAMIRTEGDQWLMHLMKDQPATGELPRGLTPMLATSGEVAGLPDSEWAFERKWDGYRLLVEIDAGEMRLRSRAGNDVTARYPQLSVLAEELADHQVILDGELIVRGPDGAVNIALLKANPRRAEFLAFDLLFLDGTSLLRKRYRDRRHVLEALAATTTELRVPPRYEGDGTEALHRSEEDGAEGVIAKRLDSVYLPGTRGHSWVKHRNWRTQEVVIGGMRRSKARPFASLLVGIPAEDGLVYAGRVGTGFDEAGMTELAARLRRSERKTPPFTNEMSADELRDAIWVTPKIKGTVRYMDWTDGGRFWHPAWLGEV